(19) United States
(12) Patent Application Publication
(10) Pub. No.: US 2026/0115178 A1
(43) Pub. Date: Apr. 30, 2026

(54) STABLE PITOLISANT LIQUID FORMULATIONS AND METHOD OF ITS USE

(71) Applicant: Sandipkumar Arvindbhai Patel, Robbinsville, NJ (US)

(72) Inventor: Sandipkumar Arvindbhai Patel, Robbinsville, NJ (US)

(21) Appl. No.: 19/370,397

(22) Filed: Oct. 27, 2025

Related U.S. Application Data
(60) Provisional application No. 63/711,702, filed on Oct. 25, 2024.
(60) Provisional application No. 63/751,532, filed on Jan. 30, 2025.
(60) Provisional application No. 63/787,829, filed on Apr. 12, 2025.
(60) Provisional application No. 63/891,890, filed on Oct. 1, 2025.

(51) Int. Cl. A61K 31/4453; A61K 47/02; A61K 47/10; A61K 47/18; A61K 47/22

(52) U.S. Cl. CPC A61K 31/4453; A61K 47/02; A61K 47/10; A61K 47/183; A61K 47/22

(57) ABSTRACT

Liquid pharmaceutical formulation of pitolisant or pharmaceutically acceptable salt thereof and its method of use are provided. The liquid formulation of pitolisant comprises Pitolisant or its pharmaceutically acceptable salt, a vehicle or solvent, and one or more optional pharmaceutically acceptable excipients. The method of preparing the liquid pitolisant formulation and the use of such formulation for the treatment of diseases treatable by pitolisant is described.

### CROSS REFERENCE TO RELATED APPLICATIONS

This application claims priority to U.S. Provisional Application No. 63/711,702 filed on Oct. 25, 2024, U.S. Provisional Application No. 63/751,532 filed on Jan. 30, 2025, U.S. Provisional Application No. 63/787,829 filed on Apr. 12, 2025, and U.S. Provisional Application No. 63/891,890 filed on Oct. 1, 2025, the disclosures of which are incorporated herein by reference in its entirety.

### FIELD OF THE INVENTION

The present invention relates to stable liquid pharmaceutical formulations of Pitolisant or its pharmaceutically acceptable salt thereof (such as Pitolisant hydrochloride) and its method of use.

### BACKGROUND OF THE INVENTION

Pitolisant is a potent and selective antagonist/inverse agonist of the histamine-3 (H3) receptor. It enhances the activity of histaminergic neurons in the brain, leading to a state of increased wakefulness and vigilance. It is the first and only non-scheduled treatment approved for treating both excessive daytime sleepiness and cataplexy in adults with narcolepsy.

Pitolisant hydrochloride is a white or almost white crystalline powder with a molecular formula of C₁₇H₂₆ClNO·HCl and a molecular weight of 332.31. The molecular formula of the pitolisant free base is C₁₇H₂₆ClNO and its molecular weight is 295.85. The chemical name of pitolisant hydrochloride is 1-{3-[3-(4-chlorophenyl) propoxy]propyl}piperidine, hydrochloride and its structural formula is:

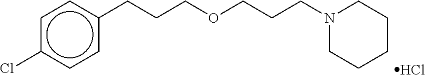

Pitolisant hydrochloride has been marketed as WAKIX® tablets since 2019 for oral administration and each film-coated tablet contains 5 mg or 20 mg of pitolisant hydrochloride (equivalent to 4.45 mg or 17.8 mg of pitolisant free base, respectively). WAKIX® tablets are indicated for the treatment of excessive daytime sleepiness (EDS) or cataplexy in adult patients with narcolepsy and treatment of excessive daytime sleepiness (EDS) in pediatric patients 6 years of age and older with narcolepsy.

Solid dosage forms of pitolisant hydrochloride, for e.g. WAKIX® tablets, lack the ease of administration in people with dysphagia (difficulty in swallowing). Such patients with dysphagia may delay taking medication or skipping the medication entirely, and thus, there is a need for alternate methods of administration. Moreover, a film-coating is applied on Pitolisant tablets to mask bitter taste of drug substances. More specifically, N-oxide derivative of Pitolisant, an oxidative degradation impurity, has very strong and prolonged bitter taste. Structure of Pitolisant N-Oxide is described below.

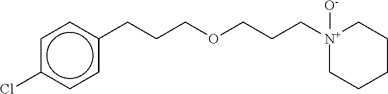

Pitolisant tablet is also being studied in phase 3 clinical for the treatment of patients with Prader-Willi Syndrome (PWS).¹ However, patients with Prader-Willi Syndrome have reported subclinical dysphagia.² Individuals with PWS may have hypotonia (low muscle tone), which affects the muscles of the mouth and throat. This can lead to a delayed swallow reflex and poor coordination. Thin liquids, like water or juice, move very quickly and can be easily aspirated (enter the airway and lungs) before the patient can trigger a safe swallow. A thicker liquid moves more slowly, giving the individual more time to control the bolus and protect their airway, significantly reducing the risk of choking and aspiration pneumonia.

The lack of child-friendly formulations leaves 40% of the world's population at increased risk for avoidable adverse events, suboptimal dosing, noncompliance, and lack of access to new medicines. A primary challenge in developing oral liquid formulations is the effective masking of drug taste, as over 90% of pediatricians in the US have indicated that taste and palatability are significant obstacles to ensuring treatment completion.³ The bitter taste found in many drugs has been suggested to result from evolutionary mechanisms that discourage consumption of potentially harmful substances.⁴ In several cases, basic taste-masking approaches may not suffice, requiring more advanced formulation strategies to encapsulate the drug and provide taste-concealing properties. Excipients utilized in product development must be confirmed as safe and suitable for use in children. These excipients are typically incorporated to optimize medication formulation by enhancing palatability, shelf-life, or manufacturing processes. ¹ https://clinicaltrials.gov/study/NCT06366464² https://pubmed.ncbi.nlm.nih.gov/27759943/³ https://pubmed.ncbi.nlm.nih.gov/19108801/⁴ https://pubmed.ncbi.nlm.nih.gov/7878094/

Taste masked stable liquid formulation of pitolisant is needed for patients with Prader-Willi Syndrome (PWS) and/or excessive daytime sleepiness (EDS) and/or cataplexy and/or narcolepsy.

Stable viscous liquid formulation of pitolisant is needed for patients with PWS.

Taste masked, and stable liquid formulation administered by oral, sublingual, buccal or intranasal route of administration is desirable. There is a need for alternate routes of administration, such as intranasal administration where the drug is delivered intranasally and reaches to the site of action, i.e. brain and have minimal systemic exposure.

There is a need for combination product of pitolisant in combination with other drug that alleviate nausea.

Critically, the approved oral tablet is associated with a significant dose-dependent systemic adverse reaction: QT interval prolongation. The official prescribing information for WAKIX® carries a warning for this effect and advises against its use in patients with known cardiac risk factors or in combination with other drugs that prolong the QT interval. The risk of this adverse reaction is directly linked to systemic concentrations of the drug, with the risk being greater in patients with hepatic or renal impairment due to higher pitolisant exposure. Clinical data show that the highest recommended oral dose increases the QTc interval, and higher exposures lead to even greater increases.

There exists a long-felt but unmet need for a liquid formulation of pitolisant. The known physicochemical properties of pitolisant and its degradation products, as disclosed in the prior art, present significant and heretofore unsolved technical challenges that have discouraged the development of a viable liquid product.

While therapeutically effective, the clinical utility of pitolisant is often limited by a significant profile of adverse events, primarily of a gastrointestinal nature. The very mechanism that confers its therapeutic benefit—an increase in systemic and central nervous system (CNS) histamine levels—is also responsible for its most common side effects.

Specifically, increased histaminergic activity stimulates H2 receptors on parietal cells in the gastric mucosa, leading to hypersecretion of gastric acid. This can cause adverse events such as stomach pain, heartburn, and gastric irritation. Furthermore, the increased histamine stimulates H1 receptors in the chemoreceptor trigger zone (CTZ) of the brainstem, a primary center for controlling nausea and emesis. This centrally mediated effect frequently leads to dose-limiting nausea in patients.

The United States Patent Application Publication no. 20250120959 described enteric coated tablet formulations that require dissolution of enteric coat followed by dissolution of core containing pitolisant hydrochloride and an alkaline agent. Dissolution of core may delay the delivery of drug to site of absorption. There is a need for enteric coated core containing liquid formulation of pitolisant that readily delivers the drug at site of absorption.

The U.S. Pat. No. 8,207,197 relates to new crystalline pitolisant monohydrochloride, the respective manufacture and methods of use, and compositions containing such a compound. The patent '197, while identifying a suitable salt form, was focused on creating a solid dosage form. The patent mentions “oral solutions” in a generic list of potential formulations but provides no working examples of a liquid. In contrast, all four detailed formulation examples (Examples 14-17) are for coated tablets, and the patent explicitly states that “more preferred forms are coated tablets”. This demonstrates that the inventors' solution to creating a viable drug product was a solid one.

United States Patent Application Publication No. US 2025/0120959 A1 demonstrates that moisture significantly contributes to pitolisant degradation, discouraging the pursuit of an aqueous liquid formulation. The '959 application reports that solid, enteric-coated tablets made via wet granulation were unstable under accelerated conditions. The inventors concluded that exposure to heat and moisture increases degradation, so a fully aqueous liquid form would likely be even less stable.

The European Public Assessment Report (EPAR) for Wakix® states that the development aim was a solid dosage form because the active substance has a “very strong and prolonged bitter taste”. The prior art's solution to this severe taste defect was a physical barrier—a film-coating—which is unavailable in a liquid solution where the drug is dissolved and in direct contact with taste receptors. Furthermore, the active substance is documented as being “sensitive to excessive oxidative conditions leading to the main identified degradation product, i.e. N-oxide derivative.” A person of ordinary skill in the art would recognize that this sensitivity to oxidation, a known problem even in the solid state, would be significantly exacerbated in liquid environment.

In light of these combined challenges—documented chemical instability expected to be worse in a liquid, a severe taste defect solved in the prior art using a tablet-only method, and the lack of any enabling disclosure for a liquid in the foundational patent—a viable, stable, and palatable oral liquid formulation of pitolisant has remained an unsolved problem and an unmet need in the art.

Current formulations do not address these mechanism-based side effects. As a result, patients may be forced to discontinue treatment or are unable to titrate to the most effective dose, leading to a suboptimal therapeutic outcome. Therefore, a significant unmet need exists for a pitolisant formulation or method of treatment that preserves its wake-promoting efficacy while mitigating or eliminating these undesirable adverse effects.

### SUMMARY OF THE INVENTION

The present invention addresses a longstanding unmet need by providing a liquid formulation of pitolisant—an objective previously unpursued by original developers due to recognized challenges associated with instability and taste.

It has been unexpectedly determined that a stable liquid formulation of pitolisant is achievable. Notably, this stability is attained despite pitolisant's documented sensitivity to oxidative conditions—a challenge that prior art indicates is particularly difficult to overcome in liquids versus solid tablets. This foundational advancement in chemical stability enables, for the first time, the development of a liquid product with a commercially viable shelf life.

Furthermore, this stable composition establishes a platform suitable for palatable oral formulations, sterile injectable formulations, and intranasal formulations for patients with unmet needs. Whereas previous approaches relied on physical film coatings to mask pitolisant's pronounced bitterness, the present invention enables the drug's delivery in a liquid format rendered palatable through the incorporation of pharmaceutically acceptable sweeteners and flavors. By resolving the primary issue of chemical stability, the invention facilitates subsequent effective mitigation of taste concerns within a liquid medium.

The present invention overcomes the limitations of the prior art by providing, for the first time, a stable pharmaceutical composition of pitolisant formulated for oral, sublingual, buccal, intranasal, and parenteral administration. The invention encompasses formulations such as solutions, or emulsion that are novel and non-obvious over the prior arts. Therefore, in an aspect of the present invention there is provided a stable liquid pharmaceutical formulation comprising: (a) pitolisant or a pharmaceutically acceptable salt thereof; and (b) one or more pharmaceutically acceptable excipients selected from the group comprising vehicles, preservatives, sweeteners, flavors, pH adjusting agents, buffering agents, viscosity adjusting agents, antioxidants, tonicity adjusting agents, chelating agents, permeability enhancing agents, mucoadhesive agents, stabilizers, complexing agents, crystallization inhibitors, emulsifying agent, suspending agents, surfactants, anti-foaming agents, dispersing agents, wetting agents, film forming agents and combinations thereof; and wherein upon storage for at least three months at room temperature, the formulation has: (i) at least about 99% pure pitolisant, (ii) less than about 1% of total degradant impurities.

In one aspect, the invention provides a pitolisant formulation with a more rapid onset of action. The intranasal formulations of the present invention are designed to provide a more rapid therapeutic effect for patients with excessive daytime sleepiness by overcoming the documented pharmacokinetic limitations of the oral tablet. Specifically, the currently approved oral tablet has a median time to maximum plasma concentration (Tₘₐₓ) of 3.5 hours, and for some patients, achieving a clinical response may take up to 8 weeks. By delivering pitolisant directly to the highly vascularized nasal mucosa, the intranasal route is designed for more rapid absorption and onset of action, thereby providing a significant clinical benefit to patients requiring prompt relief.

In another aspect, the invention provides a pitolisant formulation with an improved safety profile by mitigating the risk of systemic adverse reactions, specifically QT interval prolongation. The intranasal formulations are designed to facilitate direct nose-to-brain drug transport. This route of administration has the potential to achieve therapeutic concentrations in the CNS while requiring a lower total dose, resulting in lower peak systemic plasma concentrations (Cₘₐₓ) and overall systemic exposure (AUC) compared to the oral route. The prior art teaches that the risk of QT prolongation is directly dependent on the systemic concentration of pitolisant. A formulation that can decouple the required therapeutic CNS dose from the dose-limiting systemic concentration represents a significant and unexpected safety improvement. As the prior art provides no suggestion to use the intranasal route for pitolisant—let alone as a specific strategy to solve a dose-dependent cardiac side effect—the development of an intranasal formulation for the express purpose of enhancing the safety profile by minimizing systemic exposure and the associated risk of QT prolongation is a novel and non-obvious invention. Furthermore, the present invention provides for stable liquid intranasal formulations, overcoming the significant oxidative degradation challenges that the prior art has established as a barrier to liquid product development.

In certain embodiments, the present invention overcomes the limitations of prior art by providing a pharmaceutical composition and a method for treating narcolepsy and/or excessive daytime sleepiness that significantly reduces the incidence of gastrointestinal and centrally-mediated nausea side effects associated with pitolisant administration. The invention is based on the discovery that co-administering pitolisant with a peripherally-restricted histamine receptor antagonist can selectively block the receptors responsible for adverse effects without impairing the central H3-receptor-mediated therapeutic action.

In one embodiment, the invention describes a fixed-dose combination pharmaceutical composition comprising pitolisant or a pharmaceutically acceptable salt thereof, together with at least one peripherally-restricted histamine H2 receptor antagonist. This combination serves to reduce or prevent gastric acid hypersecretion effectively.

In another embodiment, the composition may additionally include a peripherally-selective histamine H1 receptor antagonist, such as azelastine (formulated as a nasal spray) or loratadine (formulated as an oral liquid), to alleviate nausea and vomiting signals that arise both centrally and peripherally. Importantly, this is achieved without inducing the sedation typically associated with first-generation H1 antagonists that cross the blood-brain barrier. By selectively targeting peripheral histamine receptors implicated in side effects, the compositions and methods described herein enhance the safety and tolerability profile of pitolisant, supporting improved patient compliance, a greater likelihood of reaching optimal therapeutic dosages, and overall improved clinical outcomes.

The present invention relates to liquid formulations of pitolisant or its pharmaceutically acceptable salt such as pitolisant hydrochloride, methods for their administration, processes for their manufacturing, and use of these compositions for treating diseases treatable by pitolisant.

Disclosed herein is the liquid formulation comprising pitolisant or its pharmaceutically acceptable salt thereof such as pitolisant hydrochloride and at least one or more suitable pharmaceutical excipient.

In an embodiment of the present invention, the liquid formulations of pitolisant or its pharmaceutically acceptable salt thereof, suitable for oral administration to patients in need thereof, comprises at least one or more pharmaceutically acceptable excipients selected from the group comprising, solvent, flavoring agent, sweetening agent, coloring agent, preservative, buffering agents, stabilizers, antioxidants, tonicity adjusting agent, chelating agent, pH adjusting agent, and a combination thereof.

In an embodiment of the invention, pitolisant hydrochloride is present in the liquid formulation at a concentration of about 0.1 mg/mL to about 600 mg/mL.

In an embodiment of the invention, the pitolisant hydrochloride liquid formulation is a solution.

In an embodiment, the liquid pitolisant hydrochloride formulation contains a vehicle or solvent, wherein the vehicle or solvent is water or water miscible liquid ingredient.

In an embodiment, the liquid formulation of pitolisant is prepared by solubilizing pitolisant hydrochloride in liquid solvent along with one or more pharmaceutical ingredients, filling the formulated pitolisant hydrochloride liquid into a primary packaging container, and closing the container.

In an embodiment, the liquid formulation of pitolisant is prepared by solubilizing pitolisant hydrochloride in water along with one or more pharmaceutical ingredients, filling the formulated pitolisant hydrochloride liquid into a primary packaging container, and closing the container.

In an embodiment, the liquid formulation of pitolisant suitable for oral administration is prepared by solubilizing pitolisant free base in aqueous acidic medium (such as diluted hydrochloric acid) at or below room temperature, optionally adjusting pH, followed by dissolving one or more pharmaceutical ingredients, making up the volume up to batch size using water, filling the formulated pitolisant liquid into a primary packaging container, and closing the container.

In an embodiment of the present invention, pitolisant is administered orally in the form of oral liquid, oral spray, sublingual spray or buccal spray. Such spray delivers about 5 mg to about 100 mg pitolisant hydrochloride in each spray. Volume of each oral or sublingual or buccal spray is from about 0.05 mL to 0.5 mL per spray, preferably from about 0.1 mL to 0.2 mL.

In an embodiment of the present invention, pitolisant is administered intranasally in the form of nasal spray. Such spray delivers pitolisant hydrochloride from about 0.1 mg to about 100 mg per spray. Volume of each spray is from about 0.05 mL to 0.5 mL, preferably from about 0.1 mL to 0.15 mL.

In another aspect, the invention relates to oral dosage forms wherein these stable liquid formulations are encapsulated within soft gelatin or hard gelatin capsules. Such liquid-filled capsules can be designed for immediate release or, by employing enteric coatings or specialized capsule shells (e.g., DRCaps®, ACGcaps™ HX, etc.), for delayed release, delivering the pitolisant to the intestine and reducing its side effects in stomach. Certain embodiments further comprise the inclusion of an alkaline agent (such as magnesium carbonate, potassium bicarbonate, sodium carbonate, tromethamine, meglumine, arginine, lysine, etc.) within the liquid fill formulation. These liquid-filled delayed-release capsules offer the potential advantage of faster drug availability upon dissolution of the delayed-release layer compared to conventional solid-core enteric-coated tablets, pellets or multiparticulates which require additional disintegration and/or dissolution steps.

Another aspect relates to methods of treatment for treatment of excessive daytime sleepiness (EDS) or cataplexy in adult patients with narcolepsy by administering a therapeutic effective amount of liquid pharmaceutical formulation of pitolisant hydrochloride.

Another aspect relates to methods of treatment for treatment of excessive daytime sleepiness (EDS) in pediatric patients 6 years of age and older with narcolepsy by administering a therapeutic effective amount of liquid pharmaceutical formulation of pitolisant hydrochloride.

In one aspect of the invention, the liquid formulation of pitolisant further comprises antiemetic drug selected from the group of second-generation antihistamine (H1 blocker) such as cetirizine, levocetirizine, desloratadine, fexofenadine, etc. or their pharmaceutically acceptable salt at therapeutic effective concentration.

In one aspect of the invention, the present invention provides a solution to the tolerability limitations of oral pitolisant by creating a novel pharmaceutical composition for intranasal administration designed for controlled, sustained release. The invention is predicated on the discovery that by modulating the absorption rate of pitolisant and bypassing the gastrointestinal tract, one can uncouple its therapeutic, wake-promoting effects from its dose-limiting adverse effects. One of the object of the invention is to provide a formulation that completely avoids the gastrointestinal tract. By delivering pitolisant via the nasal mucosa, the composition prevents the stimulation of gastric histamine H2 receptors, thereby eliminating side effects related to gastric acid hypersecretion, such as dyspepsia, stomach pain, and heartburn. A further object of the invention is to mitigate the centrally-mediated nausea associated with pitolisant. This is achieved by formulating the intranasal composition with one or more viscosity-enhancing agents to create a viscous liquid or gel. This viscosity increases the formulation's residence time on the nasal mucosa, creating a depot from which the pitolisant is absorbed into the systemic circulation slowly and gradually. This controlled absorption prevents the rapid spike in plasma concentration (Cₘₐₓ) that triggers nausea via stimulation of H1 receptors in the chemoreceptor trigger zone (CTZ). The resulting smooth and sustained pharmacokinetic profile maintains therapeutic plasma concentrations required for efficacy while remaining below the threshold that typically induces nausea.

In an embodiment, the present invention provides a pitolisant formulation with a superior therapeutic index, enhancing patient compliance and tolerability, and allowing for the administration of optimal therapeutic doses with a significantly reduced burden of adverse events compared to conventional oral formulations.

### BRIEF DESCRIPTION OF DRAWINGS

FIG. 1 is a bar chart representing % total impurities measured in samples from example 1 through 7 after autoclaving at about 121° C. for 15 minutes.

FIG. 2 is a bar chart representing % total impurities measured in samples from example 1, and example 8 through 15 after autoclaving at about 121° C. for 15 minutes.

FIG. 3 is a bar chart representing % total impurities measured in samples from example 1, 16, and 17 after autoclaving at about 121° C. for 15 minutes.

FIG. 4 is a line graph showing % total impurities in about 50 mg samples of pitolisant hydrochloride after treatment with increasing volumes (0 to 50 μL) of different vehicles (water, propylene glycol, castor oil, glycerin) following autoclaving at about 121° C. for 15 minutes in example 18 (A to E).

FIG. 5 is a bar chart representing % total impurities measured in samples from example 19 through 22 after autoclaving at about 121° C. for 15 minutes.

FIG. 6 is a bar chart representing % total impurities measured in samples from example 1, example 23 and 24 after autoclaving at about 121° C. for 15 minutes.

### DETAILED DESCRIPTION OF THE INVENTION

### Abbreviations

PTS: Pitolisant
PTS N-Oxide: N-Oxide derivative of pitolisant (an oxidative impurity of pitolisant)
M: Month or Months, used in stability test results as timepoints such as 1M, 2M, 3M, etc.
W: Week or Weeks, used in stability test results as timepoints such as 1W or 2W.
RT: Controlled Room Temperature (about 20-25° C.) or room temperature, used in stability test results as storage conditions, such as RT/1M that indicates when sample stored at controlled room temperature for about 1 month.
SMBS: Sodium metabisulfite.

### Definitions

As used herein, the terms “pitolisant” or “PTS” refers to pitolisant free base or its pharmaceutically acceptable salts (such as pitolisant hydrochloride), solvates, or hydrates thereof. In principle, any crystalline or amorphous form of pitolisant may be used to manufacture inventive pharmaceutical compositions of the present invention. It is understood that if the active ingredient or a portion thereof is provided as a salt, the concentration of active ingredient salt may be higher than the concentration of active ingredient. For example, a pharmaceutical composition comprising pitolisant at a concentration of about 4.45 mg/mL may comprise pitolisant hydrochloride at a concentration of about 5 mg/mL. It is also understood that the concentration of active ingredient salt may be selected to provide active ingredient concentration as described herein.

As used herein, the terms “about” or “approximately” or symbol “˜” refer to within a range of ±20%, optionally within ±10%, optionally within ±5%, optionally within ±3%, optionally within ±2%, optionally within ±1%, optionally within ±0.5%, optionally within ±0.1%, or optionally within ±0.01% of the stated value, except where such number would be less than 0% or exceed 100% of a possible value. As used herein, reference to “approximately” or “about” a value or parameter includes (and describes) embodiments that are directed to that value or parameter. For example, description referring to “about X” includes description of “X”.

As used herein, the term “or” means “and/or.” The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the terms “composition” or “formulation” may be used interchangeably and refer to the liquid pharmaceutical compositions of the present invention. The liquid formulation of the present invention may be delivered in different dosage forms (a physical form of preparation) such as solution, emulsion, suspension, liquid-filled capsule, depot injection, etc. The liquid formulation of the present invention may be delivered in the form of dosage form suitable for immediate release or modified release such as sustained release or delayed release. The scope of the invention relies, without wishing to be bound with any theory, wherever the pitolisant is primarily in dissolved or liquid state within the pharmaceutical carrier.

The term “purity” describes the proportion of pitolisant or its salt in its original form, expressed as a percentage (%). Purity is determined by calculating the area percentage of the pitolisant peak (main peak or parent peak) when analyzed using an appropriate chromatographic method such as HPLC. The terms “impurity” or “degradation impurity” or “pitolisant-related impurity” used herein refer to any chemical compound that is undesired and generated from the degradation of pitolisant during the formulation process or storage of the formulation. A specific impurity of notes is pitolisant N-oxide or PTS N-oxide or N-oxide derivative of pitolisant. The concentrations of these compounds are reported as a percentage (%), representing the area percentage of the impurity peak relative to the pitolisant peak when analyzed by a suitable chromatographic method such as HPLC. Such impurities may be reported individually or in total. The term “highest single impurity” or “maximum single impurity” or “maximum individual impurity” or “highest individual impurity” refers to any individual impurity with the highest percent peak area. The term “total impurities” refers to the combined amount of all impurities separated from pitolisant peak when analyzed using HPLC and concentration of total impurities is reported in percentage (%) area. An alternative method for determining total impurities is to subtract the percentage of purity from 100.

The United States Pharmacopoeia (USP) defines stability as the ability of a product to retain its characteristics that it possessed during its manufacturing within specified limits throughout its period of storage and use. According to the ICH guidelines, pharmaceutical stability testing is defined as systematic experiments conducted on pharmaceutical products to understand and provide evidence how the quality of a drug product varies under the influence of a variety of environmental factors such as temperature, and to set a re-test period for the drug, or a shelf life for the drug product and recommend good storage conditions. In view of these, degradation products/impurities in a drug product are required to be evaluated and reported to the regulatory agencies. The term “stable” used herein refers to the invented composition that has less 10%, preferably less than 5%, preferably less than 3%, preferably less than 2% of the total degradation impurities after extended period at room temperature (15-25° C.), or at refrigerated condition, i.e. 2-8° C. In some embodiments, the invented composition that has less than 10%, preferably less than 5%, preferably less than 3%, preferably less than 2%, preferably less than 1% of the total degradation impurities after shorter period at accelerated conditions such as about 40° C., about 60° C., about 70° C., or about 120° C. (such as terminal sterilization for about 5 to 15 minutes) may also be considered stable.

As used herein, the term “shelf life” refers to the length of time that a formulation may be stored without becoming unfit for medical use. Examples of compositions which are unfit for medical use include, but are not limited to, compositions with unacceptably high impurity levels (such as more than 10% total impurities or less than 90% purity), and/or the presence of physical changes such as precipitation or discoloration.

As used herein, the term “step” is not particularly limiting. For example, each step as described herein may be a discrete step such that steps are performed sequentially upon completion of a preceding step. Alternatively, at least a portion of the method may be continuous, or the steps may be performed out of the order stated.

It should be noted that the unit of measurement for the quantity of any ingredients in this invention, such as “%”, typically represents % w/w. In certain embodiments, this quantity may also be expressed using the unit % w/v. If the units are presented in % w/w in the present disclosure, it is also considered as % w/v, if the difference in concentration resulting from the conversion is less than about 10%. It is also understood that units presented in “mg/mL” or “mg per mL” can be converted into “% w/v” using this formula: % w/v=[mg/mL]×0.1; or mg/mL=[% w/v]×10.

As used herein, the term “parenteral”, refers to the route of administration of the formulation of the present invention other than topical or oral. Examples of parenteral routes include intramuscular, intravascular (including intraarterial or intravenous), intraorbital, retrobulbar, intranasal, intrathecal, intraventricular, intraspinal, intraperitoneal, intrapulmonary, intracisternal, intracapsular, intrasternal, peribulbar, or intralesional administration.

As used herein, the terms “micron”, or “microns” or “micrometer” or “μm” refer to one millionth of a meter.

As used in this document, the term “oral spray” denotes a liquid formulation designed for administration as a spray within the oral cavity or a specific region thereof. Specific sites within the oral cavity include the buccal, sublingual, gingival, or general mouth area. In certain embodiments, delivery to the sublingual, buccal, or gingival regions may facilitate enhanced or more efficient absorption relative to other areas of the oral cavity. Oral liquid formulations can be administered as an oral spray, oral solution, or as a stream or jet of liquid that does not form a spray but can be administered directly. Some embodiments of the present invention, the pitolisant liquid formulation suitable for oral use or oral administration may be oral liquid and/or oral spray.

As used herein, the term “liquid carrier” refers to a liquid medium comprising one or more pharmaceutically acceptable excipients or inactive ingredients described in this disclosure.

As used herein, the term “delayed release” refers to a formulation or dosage form that is designed to release the active pharmaceutical ingredient at a time other than immediately after administration. This means the drug is released at a later point, often after passing through the stomach or upon reaching a specific part of the gastrointestinal tract, in order to achieve targeted absorption or reduce potential side effects.

The term “stabilizing amount” refers to the concentration of an ingredient that enables the formulation of a stable liquid containing pitolisant or its pharmaceutically acceptable salts. In some cases, certain concentrations of pitolisant or its pharmaceutically acceptable salt may result in self-stabilization without a stabilizer. In other cases, additional ingredients such as a chelating agent or stabilizer may be required to maintain stability when the concentration of pitolisant is higher or does not remain stable without a stabilizer.

The term “nitrogen overlay” refers to purging nitrogen in head space of liquid filled container before closing the closure. The nitrogen or other inert gas such as helium or argon is purged to replace headspace oxygen and reduce oxygen concentration in headspace lesser than atmospheric concentration (i.e. about 21%). In an embodiment of the present invention, nitrogen overlay may not be required if the liquid is filled to about the full capacity of the container or the concentration of pitolisant is low enough for this degradation to occur or occur at lower rate. In another embodiment, nitrogen is required to reduce the headspace oxygen concentration less than 20%, more preferably less than 15%, more preferably less than 10%, more preferably less than 5%, and most preferably less than 2%.

The present invention relates to a stable liquid pharmaceutical formulation comprising: (a) pitolisant or a pharmaceutically acceptable salt thereof; (b) one or more pharmaceutically acceptable excipients selected from the group comprising vehicles, preservatives, sweeteners, flavors, pH adjusting agents, buffering agents, viscosity adjusting agents, antioxidants, tonicity adjusting agents, chelating agents, permeability enhancing agents, mucoadhesive agents, stabilizers, complexing agents, crystallization inhibitors, emulsifying agent, suspending agents, surfactants, anti-foaming agents, dispersing agents, wetting agents, film forming agent, and combinations thereof; and wherein upon storage for at least three months at room temperature, the formulation has: (i) at least about 99% pure pitolisant, (ii) less than about 1% of total degradant impurities.

In an embodiment, the invention provides stable liquid pharmaceutical compositions of pitolisant or its pharmaceutically acceptable salt thereof such as pitolisant hydrochloride, wherein pitolisant is present at a concentration in the range from about 0.1 mg/mL to about 900 mg/mL, optionally from about 0.1 mg/mL to about 400 mg/mL.

In some embodiments, the present invention provides liquid pharmaceutical formulations, wherein the concentration of pitolisant hydrochloride is about 0.1 mg/mL, about 0.2 mg/mL, about 0.3 mg/mL, about 0.4 mg/mL, about 0.5 mg/mL, about 0.7 mg/mL, about 1 mg/mL, about 1.2 mg/mL, about 1.5 mg/mL, about 2 mg/mL, about 2.5 mg/mL, about 3 mg/mL, about 3.5 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, about 50 mg/mL, about 51 mg/mL, about 52 mg/mL, about 53 mg/mL, about 54 mg/mL, about 55 mg/mL, about 56 mg/mL, about 57 mg/mL, about 58 mg/mL, about 59 mg/mL, about 60 mg/mL, about 61 mg/mL, about 62 mg/mL, about 63 mg/mL, about 64 mg/mL, about 65 mg/mL, about 66 mg/mL, about 67 mg/mL, about 68 mg/mL, about 69 mg/mL, about 70 mg/mL, about 71 mg/mL, about 72 mg/mL, about 73 mg/mL, about 74 mg/mL, about 75 mg/mL, about 76 mg/mL, about 77 mg/mL, about 78 mg/mL, about 79 mg/mL, about 80 mg/mL, about 81 mg/mL, about 82 mg/mL, about 83 mg/mL, about 84 mg/mL, about 85 mg/mL, about 86 mg/mL, about 87 mg/mL, about 88 mg/mL, about 89 mg/mL, about 90 mg/mL, about 91 mg/mL, about 92 mg/mL, about 93 mg/mL, about 94 mg/mL, about 95 mg/mL, about 96 mg/mL, about 97 mg/mL, about 98 mg/mL, about 99 mg/mL, about 100 mg/mL, about 101 mg/mL, about 102 mg/mL, about 103 mg/mL, about 104 mg/mL, about 105 mg/mL, about 106 mg/mL, about 107 mg/mL, about 108 mg/mL, about 109 mg/mL, about 110 mg/mL, about 111 mg/mL, about 112 mg/mL, about 113 mg/mL, about 114 mg/mL, about 115 mg/mL, about 116 mg/mL, about 117 mg/mL, about 118 mg/mL, about 119 mg/mL, about 120 mg/mL, about 121 mg/mL, about 122 mg/mL, about 123 mg/mL, about 124 mg/mL, about 125 mg/mL, about 126 mg/mL, about 127 mg/mL, about 128 mg/mL, about 129 mg/mL, about 130 mg/mL, about 131 mg/mL, about 132 mg/mL, about 133 mg/mL, about 134 mg/mL, about 135 mg/mL, about 136 mg/mL, about 137 mg/mL, about 138 mg/mL, about 139 mg/mL, about 140 mg/mL, about 141 mg/mL, about 142 mg/mL, about 143 mg/mL, about 144 mg/mL, about 145 mg/mL, about 146 mg/mL, about 147 mg/mL, about 148 mg/mL, about 149 mg/mL, about 150 mg/mL, about 151 mg/mL, about 152 mg/mL, about 153 mg/mL, about 154 mg/mL, about 155 mg/mL, about 156 mg/mL, about 157 mg/mL, about 158 mg/mL, about 159 mg/mL, about 160 mg/mL, about 161 mg/mL, about 162 mg/mL, about 163 mg/mL, about 164 mg/mL, about 165 mg/mL, about 166 mg/mL, about 167 mg/mL, about 168 mg/mL, about 169 mg/mL, about 170 mg/mL, about 171 mg/mL, about 172 mg/mL, about 173 mg/mL, about 174 mg/mL, about 175 mg/mL, about 176 mg/mL, about 177 mg/mL, about 178 mg/mL, about 179 mg/mL, about 180 mg/mL, about 181 mg/mL, about 182 mg/mL, about 183 mg/mL, about 184 mg/mL, about 185 mg/mL, about 186 mg/mL, about 187 mg/mL, about 188 mg/mL, about 189 mg/mL, about 190 mg/mL, about 191 mg/mL, about 192 mg/mL, about 193 mg/mL, about 194 mg/mL, about 195 mg/mL, about 196 mg/mL, about 197 mg/mL, about 198 mg/mL, about 199 mg/mL, about 200 mg/mL, about 201 mg/mL, about 202 mg/mL, about 203 mg/mL, about 204 mg/mL, about 205 mg/mL, about 206 mg/mL, about 207 mg/mL, about 208 mg/mL, about 209 mg/mL, about 210 mg/mL, about 211 mg/mL, about 212 mg/mL, about 213 mg/mL, about 214 mg/mL, about 215 mg/mL, about 216 mg/mL, about 217 mg/mL, about 218 mg/mL, about 219 mg/mL, about 220 mg/mL, about 221 mg/mL, about 222 mg/mL, about 223 mg/mL, about 224 mg/mL, about 225 mg/mL, about 226 mg/mL, about 227 mg/mL, about 228 mg/mL, about 229 mg/mL, about 230 mg/mL, about 231 mg/mL, about 232 mg/mL, about 233 mg/mL, about 234 mg/mL, about 235 mg/mL, about 236 mg/mL, about 237 mg/mL, about 238 mg/mL, about 239 mg/mL, about 240 mg/mL, about 241 mg/mL, about 242 mg/mL, about 243 mg/mL, about 244 mg/mL, about 245 mg/mL, about 246 mg/mL, about 247 mg/mL, about 248 mg/mL, about 249 mg/mL, about 250 mg/mL, about 251 mg/mL, about 252 mg/mL, about 253 mg/mL, about 254 mg/mL, about 255 mg/mL, about 256 mg/mL, about 257 mg/mL, about 258 mg/mL, about 259 mg/mL, about 260 mg/mL, about 261 mg/mL, about 262 mg/mL, about 263 mg/mL, about 264 mg/mL, about 265 mg/mL, about 266 mg/mL, about 267 mg/mL, about 268 mg/mL, about 269 mg/mL, about 270 mg/mL, about 271 mg/mL, about 272 mg/mL, about 273 mg/mL, about 274 mg/mL, about 275 mg/mL, about 276 mg/mL, about 277 mg/mL, about 278 mg/mL, about 279 mg/mL, about 280 mg/mL, about 281 mg/mL, about 282 mg/mL, about 283 mg/mL, about 284 mg/mL, about 285 mg/mL, about 286 mg/mL, about 287 mg/mL, about 288 mg/mL, about 289 mg/mL, about 290 mg/mL, about 291 mg/mL, about 292 mg/mL, about 293 mg/mL, about 294 mg/mL, about 295 mg/mL, about 296 mg/mL, about 297 mg/mL, about 298 mg/mL, about 299 mg/mL, about 300 mg/mL, about 301 mg/mL, about 302 mg/mL, about 303 mg/mL, about 304 mg/mL, about 305 mg/mL, about 306 mg/mL, about 307 mg/mL, about 308 mg/mL, about 309 mg/mL, about 310 mg/mL, about 311 mg/mL, about 312 mg/mL, about 313 mg/mL, about 314 mg/mL, about 315 mg/mL, about 316 mg/mL, about 317 mg/mL, about 318 mg/mL, about 319 mg/mL, about 320 mg/mL, about 321 mg/mL, about 322 mg/mL, about 323 mg/mL, about 224 mg/mL, about 325 mg/mL, about 326 mg/mL, about 327 mg/mL, about 328 mg/mL, about 329 mg/mL, about 330 mg/mL, about 331 mg/mL, about 332 mg/mL, about 333 mg/mL, about 334 mg/mL, about 335 mg/mL, about 336 mg/mL, about 337 mg/mL, about 338 mg/mL, about 339 mg/mL, about 340 mg/mL, about 341 mg/mL, about 342 mg/mL, about 343 mg/mL, about 344 mg/mL, about 345 mg/mL, about 346 mg/mL, about 347 mg/mL, about 348 mg/mL, about 349 mg/mL, about 350 mg/mL, about 351 mg/mL, about 352 mg/mL, about 353 mg/mL, about 354 mg/mL, about 355 mg/mL, about 356 mg/mL, about 357 mg/mL, about 358 mg/mL, about 359 mg/mL, about 360 mg/mL, about 361 mg/mL, about 362 mg/mL, about 363 mg/mL, about 364 mg/mL, about 365 mg/mL, about 366 mg/mL, about 367 mg/mL, about 368 mg/mL, about 369 mg/mL, about 370 mg/mL, about 371 mg/mL, about 372 mg/mL, about 373 mg/mL, about 374 mg/mL, about 375 mg/mL, about 376 mg/mL, about 377 mg/mL, about 378 mg/mL, about 379 mg/mL, about 380 mg/mL, about 381 mg/mL, about 382 mg/mL, about 383 mg/mL, about 384 mg/mL, about 385 mg/mL, about 386 mg/mL, about 387 mg/mL, about 388 mg/mL, about 389 mg/mL, about 390 mg/mL, about 391 mg/mL, about 392 mg/mL, about 393 mg/mL, about 394 mg/mL, about 395 mg/mL, about 396 mg/mL, about 397 mg/mL, about 398 mg/mL, about 399 mg/mL, about 400 mg/mL, about 500 mg/mL, about 600 mg/mL, about 800 mg/mL, about 900 mg/mL. The concentration of pitolisant hydrochloride may be from about 0.1 mg/mL to about 900 mg/mL, from about 5 mg/mL to about 200 mg/mL, from about 5 mg/mL to about 160 mg/mL, from about 5 mg/mL to 50 mg/mL.

In an embodiment of the present invention, the liquid pharmaceutical formulation of pitolisant may be provided as solution, suspension, or emulsion, preferably as a solution or emulsion, more preferably as a solution.

In one embodiment, the formulation is suitable for administration selected from oral, sublingual, buccal or intranasal routes.

In another embodiment, the formulation is sterile and suitable for parenteral or injectable administration.

### Oral Formulations

In an embodiment of the present invention, the stable liquid pharmaceutical formulation of pitolisant or a pharmaceutically acceptable salt thereof is provided in a dosage form, such as solution, emulsion, suspension, or liquid filled solid dosage forms suitable for oral administration.

In an embodiment, the present invention provides a liquid pharmaceutical formulation suitable for oral administration, wherein the formulation comprises (i) pitolisant or a pharmaceutically acceptable salt thereof at a concentration in the range from about 0.1 mg/mL to about 400 mg/mL, and (ii) at least one pharmaceutically acceptable vehicle.

In an embodiment, the present invention provides a liquid pharmaceutical formulation suitable for oral administration, wherein the formulation comprises (i) pitolisant or a pharmaceutically acceptable salt thereof at a concentration in the range from about 0.1 mg/mL to about 400 mg/mL, and (ii) at least one pharmaceutically acceptable excipient; and (iii) at least one pharmaceutically acceptable vehicle.

In an embodiment, the present invention provides a liquid pharmaceutical formulation suitable for oral administration, wherein the formulation comprises (i) pitolisant or a pharmaceutically acceptable salt thereof at a concentration in the range from about 0.1 mg/mL to about 400 mg/mL, and (ii) at least one pharmaceutically acceptable excipient selected from the group comprising of preservatives, sweeteners, flavors, pH adjusting agents, buffering agents, viscosity adjusting agents, antioxidants, tonicity adjusting agents, chelating agents, permeation enhancers, mucoadhesive agents, stabilizers, complexing agents, crystallization inhibitors, emulsifying agents, suspending agents, surfactants, anti-foaming agents, dispersing agents, and wetting agents; and (iii) at least one pharmaceutically acceptable vehicle.

In an embodiment, the present invention provides a liquid pharmaceutical formulation suitable for oral administration, wherein the formulation comprises (i) pitolisant or a pharmaceutically acceptable salt thereof at a concentration in the range from about 0.1 mg/mL to about 400 mg/mL, and (ii) at least one pharmaceutically acceptable excipient selected from the group comprising of preservatives, sweeteners, flavors, pH adjusting agents, buffering agents, viscosity adjusting agents, antioxidants, tonicity adjusting agents, chelating agents, permeation enhancers, mucoadhesive agents, stabilizers, complexing agents, crystallization inhibitors, emulsifying agents, suspending agents, surfactants, anti-foaming agents, dispersing agents, wetting agents, and film forming agents; and (iii) at least one pharmaceutically acceptable vehicle which is water.

In one embodiment of the present invention, pitolisant or a pharmaceutically acceptable salt thereof may be present at a concentration in the range from about 4 mg/mL to about 40 mg/mL.

In one embodiment of the present invention, pitolisant or a pharmaceutically acceptable salt thereof may be present at a concentration of about 20 mg/mL.

In an embodiment of the present invention, the pitolisant is present in the formulation in the form of pitolisant hydrochloride. In an embodiment, the present invention provides a liquid pharmaceutical formulation of pitolisant suitable for oral administration, wherein the formulation contains a preservative and intended for unit dose or multiple dose administration, preferably multiple dose administration.

In an embodiment, the present invention provides a liquid pharmaceutical formulation filled in soft gelatin or hard gelatin capsule suitable for oral administration, wherein the formulation comprises (i) pitolisant or a pharmaceutically acceptable salt thereof at a concentration in the range from about 0.1 mg/mL to about 600 mg/mL; (ii) at least one pharmaceutically acceptable vehicle; and (iii) a cavity forming polymer.

In an embodiment, the present invention provides a liquid pharmaceutical formulation filled in soft gelatin or hard gelatin capsule suitable for oral administration, wherein the formulation comprises (i) pitolisant or a pharmaceutically acceptable salt thereof at a concentration in the range from about 0.1 mg/mL to about 600 mg/mL; (ii) at least one pharmaceutically acceptable vehicle; and (iii) a cavity forming polymer with delayed release properties.

In an embodiment of the present invention, the liquid formulation of pitolisant suitable for oral administration is solution comprising pitolisant hydrochloride (from about 0.1% w/v to about 40% w/v, preferably about 0.4% w/v to about 5% w/v), vehicle (q.s. to 100% w/v) and one or more optional ingredients selected from group comprising of sweetener (from about 0.1% w/v to about 70% w/v, preferably from about 0.1% w/v to about 5% w/v), pH adjusting agent (q.s. to adjust pH between about 2 and about 7, optionally between about 2 and about 6), stabilizer (from about 0.01% w/v to about 1% w/v, preferably from about 0.1% w/v to about 1% w/v), surfactant (from about 0.01% w/v to about 1% w/v, preferably from about 0.01% w/v to about 0.2% w/v), preservative (from about 0.01% w/v to about 4% w/v), antioxidant (from about 0.01% w/v to about 0.5% w/v), viscosity adjusting agent (from about 0.01% w/v to about 20% w/v, preferably from about 0.1% w/v to about 5% w/v), and flavoring agent (from about 0.001% w/v to about 2% w/v).

In an embodiment of the present invention, the liquid formulation of pitolisant suitable for oral administration is solution comprising pitolisant hydrochloride (from about 0.4% w/v to about 5% w/v), vehicle (q.s. to 100% w/v) and one or more optional ingredient selected from group comprising of sweetener (from about 0.1% w/v to about 5% w/v), pH adjusting agent (q.s. to adjust pH between about 2 and about 6), stabilizer (from about 0.01% w/v to about 1% w/v, preferably from about 0.1% to about 0.5%, preferably from about 0.1% to about 0.25%), viscosity adjusting agent (from about 0.1% w/v to about 5% w/v), preservative (from about 0.01% w/v to about 1% w/v), antioxidant (from about 0.001% w/v to about 0.2% w/v), and flavoring agent (from about 0.01% w/v to about 1% w/v).

In an embodiment of the present invention, the liquid formulation of pitolisant suitable for oral administration is solution comprising pitolisant hydrochloride (from about 0.4% w/v to about 20% w/v), sucralose (from about 0.1% w/v to about 5% w/v), sodium benzoate (from about 0.01% w/v to about 1% w/v), hydrochloric acid and/or sodium hydroxide (0.1N) optionally used to adjust the pH between about 3 and about 5, cherry flavor (from about 0.01% w/v to about 1% w/v), and purified water (q.s. to 100% w/v).

In an embodiment of the present invention, the liquid formulation of pitolisant suitable for oral administration is solution comprising pitolisant hydrochloride (from about 0.4% w/v to about 20% w/v), sucralose (from about 0.1% w/v to about 5% w/v), hydroxypropyl methylcellulose or hypromellose (from about 0.01% w/v to about 2% w/v), sodium benzoate (from about 0.01% w/v to about 1% w/v), hydrochloric acid and/or sodium hydroxide (0.1N) optionally used to adjust the pH between about 3 and about 5, cherry flavor (from about 0.01% w/v to about 1% w/v), and purified water (q.s. to 100% w/v).

In an embodiment of the present invention, the liquid formulation of pitolisant suitable for oral administration is solution comprising pitolisant hydrochloride (from about 0.4% w/v to about 20% w/v), sucralose (from about 0.1% w/v to about 5% w/v), hydroxypropyl methylcellulose or hypromellose K4M (from about 0.05% w/v to about 2% w/v), sodium benzoate (from about 0.01% w/v to about 1% w/v), hydrochloric acid and/or sodium hydroxide (0.1N) optionally used to adjust the pH between about 3 and about 5, cherry flavor (from about 0.01% w/v to about 1% w/v), and purified water (q.s. to 100% w/v).

In an embodiment of the present invention, the liquid formulation of pitolisant suitable for oral administration is solution comprising pitolisant hydrochloride (from about 0.4% w/v to about 20% w/v), sucralose (from about 0.1% w/v to about 5% w/v), hydroxypropyl methylcellulose or hypromellose K4M (from about 0.05% w/v to about 2% w/v), methylparaben (about 0.1% w/v to about 0.2% w/v), propylparaben (from about 0.01% w/v to about 0.04% w/v), hydrochloric acid and/or sodium hydroxide (0.1N) optionally used to adjust the pH between about 3 and about 5.5, cherry flavor (from about 0.01% w/v to about 1% w/v), and purified water (q.s. to 100% w/v).

In an embodiment of the present invention, the liquid formulation of pitolisant suitable for oral administration is oil-in-water emulsion comprising pitolisant (from about 0.1% w/v to about 40% w/v, preferably about 0.4% w/v to about 5% w/v), vehicle (q.s. to 100% w/v) and one or more ingredients selected from group comprising of sweetener (from about 0.1% w/v to about 70% w/v, preferably from about 0.1% w/v to about 5% w/v), pH adjusting agent (q.s. to adjust pH between about 7 and about 9, optionally between about 8 and about 9), stabilizer (from about 0.01% w/v to about 1% w/v, preferably from about 0.1% w/v to about 1% w/v), surfactant (from about 0.01% w/v to about 1% w/v, preferably from about 0.01% w/v to about 0.2% w/v), preservative (from about 0.01% w/v to about 4% w/v), antioxidant (from about 0.01% w/v to about 0.5% w/v), viscosity adjusting agent (from about 0.01% w/v to about 20% w/v, preferably from about 0.1% w/v to about 5% w/v), emulsifying agent (from about 0.01% w/v to about 5% w/v), and flavoring agent (from about 0.001% w/v to about 2% w/v).

In an embodiment, the liquid formulation of pitolisant suitable for oral administration is suspension comprising pitolisant (from about 0.1% w/v to about 20% w/v), vehicle (q.s. to 100% w/v) and one or more ingredients selected from group comprising of suspending agent (from about 0.1% w/v to about 4% w/v), surfactant (from about 0.01% to about 2%), sweetener (from about 0.1% w/v to about 40% w/v, preferably from about 0.1% w/v to about 5% w/v), viscosity adjusting agent (from about 0.1% w/v to about 5% w/v, preferably from about 0.1% to about 2%, preferably from about 0.1% to 0.5%), pH adjusting agent (q.s. to adjust pH between about 7 and about 10), stabilizer (from about 0.01% w/v to about 1% w/v), preservative (from about 0.01% w/v to about 1% w/v), antioxidant (from about 0.01% w/v to about 0.5% w/v), and flavoring agent (from about 0.01% w/v to about 1% w/v).

In an embodiment, the liquid formulation of pitolisant suitable for oral administration is suspension comprising pitolisant (from about 0.4% w/v to about 5% w/v), vehicle (q.s. to 100% w/v) and one or more ingredients selected from group comprising of suspending agent (from about 0.1% w/v to about 2% w/v), viscosity adjusting agent (from about 0.1% w/v to about 4% w/v), surfactant (from about 0.01% w/v to about 0.5% w/v), sweetener (from about 0.1% w/v to about 30% w/v, preferably from about 0.1% w/v to about 2% w/v), pH adjusting agent (q.s. to adjust pH between about 7 and about 10), stabilizer (from about 0.01% w/v to about 1% w/v), preservative (from about 0.01% w/v to about 1% w/v), antioxidant (from about 0.01% w/v to about 0.5% w/v), and flavoring agent (from about 0.01% w/v to about 1% w/v).

In an embodiment of the present invention, the liquid pharmaceutical formulation suitable for oral administration filled in delayed release capsules comprises pitolisant or its pharmaceutically acceptable salt, preferably pitolisant hydrochloride (from about 0.1% w/v to about 60% w/v), a cavity forming polymer with delayed release properties (from about 0.05% w/v to about 10% w/w), a pharmaceutically acceptable vehicle (q.s. to 100% w/w), and one or more optional ingredients selected from the group comprising, preservative (from about 0.01% w/v to about 1% w/v), and antioxidant (from about 0.01% w/v to about 0.5% w/v).

### Intranasal Formulations

In an embodiment of the present invention, the stable liquid pharmaceutical formulation of pitolisant or a pharmaceutically acceptable salt thereof is provided in a dosage form, such as solution, emulsion or suspension, suitable for intranasal administration.

In an embodiment, the present invention provides a liquid pharmaceutical formulation suitable for intranasal administration, wherein the composition comprises (i) pitolisant or a pharmaceutically acceptable salt thereof at a concentration in the range from about 1 mg/mL to about 800 mg/mL, optionally from about 10 mg/mL to about 400 mg/mL; and (ii) at least one pharmaceutically acceptable vehicle.

In an embodiment, the present invention provides a liquid pharmaceutical formulation suitable for intranasal administration, wherein the composition comprises (i) pitolisant or a pharmaceutically acceptable salt thereof at a concentration in the range from about 1 mg/mL to about 800 mg/mL, optionally from about 10 mg/mL to about 400 mg/mL; and (ii) at least one pharmaceutically acceptable excipient; and (iii) at least one pharmaceutically acceptable vehicle.

In an embodiment, the present invention provides a liquid pharmaceutical formulation suitable for intranasal administration, wherein the composition comprises (i) pitolisant or a pharmaceutically acceptable salt thereof at a concentration in the range from about 1 mg/mL to about 800 mg/mL, optionally from about 10 mg/mL to about 400 mg/mL; and (ii) at least one pharmaceutically acceptable excipient selected from group comprising of preservatives, sweeteners, flavors, pH adjusting agents, buffering agents, viscosity adjusting agents, antioxidants, tonicity adjusting agents, chelating agents, permeation enhancers, mucoadhesive agents, stabilizers, complexing agents, crystallization inhibitors, emulsifying agents, suspending agents, surfactants, anti-foaming agents, dispersing agents, and wetting agents; and (iii) at least one pharmaceutically acceptable vehicle.

In an embodiment, the present invention provides a liquid pharmaceutical formulation suitable for intranasal administration, wherein the composition comprises (i) pitolisant or a pharmaceutically acceptable salt thereof at a concentration in the range from about 1 mg/mL to about 400 mg/mL, and (ii) at least one pharmaceutically acceptable excipient selected from group comprising of preservatives, sweeteners, flavors, pH adjusting agents, buffering agents, viscosity adjusting agents, antioxidants, tonicity adjusting agents, chelating agents, permeation enhancers, mucoadhesive agents, stabilizers, complexing agents, crystallization inhibitors, emulsifying agents, suspending agents, surfactants, anti-foaming agents, dispersing agents, and wetting agents; and (iii) at least one pharmaceutically acceptable vehicle which is water.

In another embodiment of the present invention, pitolisant or a pharmaceutically acceptable salt thereof may be present at a concentration in the range from about 10 mg/mL to about 200 mg/mL.

In an embodiment of the present invention, the pitolisant is present in the formulation in the form of pitolisant hydrochloride.

In an embodiment, the present invention provides a liquid pharmaceutical formulation of pitolisant suitable for intranasal administration, wherein the formulation is sterile, preservative free intended for unit dose or bi-dose or multiple dose administration.

In an embodiment, the present invention provides a liquid pharmaceutical formulation of pitolisant suitable for intranasal administration, wherein the formulation contains a preservative and intended for unit dose or bi-dose or multiple dose administration, preferably multiple dose administration.

In an embodiment of the present invention, the liquid formulation of pitolisant suitable for intranasal administration is solution comprising pitolisant hydrochloride (from about 1% w/v to about 40% w/v, preferably about 1% w/v to about 20% w/v), vehicle (q.s. to 100% w/v) and one or more ingredients selected from group comprising of preservative (from about 0.001% w/v to about 0.5% w/v), viscosity adjusting agent (from about 0.001% w/v to about 1% w/v, preferably from about 0.01% w/v to about 0.5% w/v), chelating agent (from about 0.001% w/v to about 1% w/v), sweetener (from about 0.1% w/v to about 5% w/v, preferably from about 0.1% w/v to about 1% w/v), pH adjusting agent (q.s. to adjust pH between about 3 and about 7, optionally between about 4 and about 6), stabilizer (from about 0.001% w/v to about 1% w/v, preferably from about 0.01% w/v to about 0.5% w/v), surfactant (from about 0.01% w/v to about 1% w/v, preferably from about 0.01% w/v to about 0.2% w/v), antioxidant (from about 0.01% w/v to about 0.5% w/v), flavoring agent (from about 0.001% w/v to about 2% w/v), permeability enhancing agent (from about 0.01% w/v to about 1% w/v), and tonicity adjusting agent (from about 0.1% w/v to about 5% w/v).

In an embodiment of the present invention, the liquid formulation of pitolisant suitable for intranasal administration is solution comprising pitolisant hydrochloride (from about 5% w/v to about 20% w/v), vehicle (q.s. to 100% w/v) and one or more optional ingredients selected from group comprising of preservative (from about 0.001% w/v to about 0.5% w/v), viscosity adjusting agent (from about 0.01% w/v to about 0.5% w/v), chelating agent (from about 0.01% w/v to about 1% w/v), sweetener (from about 0.1% w/v to about 1% w/v), pH adjusting agent (optional, q.s. to adjust pH between about 4 and about 6), and permeability enhancing agent (from about 0.01% w/v to about 1% w/v).

In an embodiment of the present invention, the liquid formulation of pitolisant suitable for intranasal administration is solution comprising pitolisant hydrochloride (from about 5% w/v to about 20% w/v), benzalkonium chloride (from about 0.01% w/v to about 1% w/v), hydroxypropyl methylcellulose or hypromellose K4M (from about 0.05% w/v to about 0.5% w/v), edetate disodium (from about 0.01% w/v to about 1% w/v), sucralose (from about 0.1% w/v to about 1% w/v), hydrochloric acid and/or sodium hydroxide (optional, q.s. to adjust pH between about 4 and about 6, and purified water (q.s. to 100% w/v).

In an embodiment of the present invention, the liquid formulation of pitolisant suitable for intranasal administration is solution comprising pitolisant hydrochloride (from about 5% w/v to about 20% w/v), benzalkonium chloride (from about 0.01% w/v to about 1% w/v), hydroxypropyl methylcellulose or hypromellose K4M (from about 0.05% w/v to about 0.5% w/v), edetate disodium (from about 0.01% w/v to about 1% w/v), sucralose (from about 0.1% w/v to about 1% w/v), hydrochloric acid and/or sodium hydroxide (optional, q.s. to adjust pH between about 4 and about 6, n-dodecyl beta-D-maltoside (from about 0.1% w/v to about 1% w/v, preferably from about 0.2% w/v to about 0.5% w/v), and purified water (q.s. to 100% w/v).

In an embodiment of the present invention, the liquid formulation of pitolisant suitable for intranasal administration is oil-in-water emulsion comprising pitolisant (from about 0.1% w/v to about 40% w/v, preferably about 0.4% w/v to about 5% w/v), vehicle (q.s. to 100% w/v) and one or more ingredients selected from group comprising of sweetener (from about 0.1% w/v to about 70% w/v, preferably from about 0.1% w/v to about 5% w/v), tonicity adjusting agent (from about 0.1% w/v to about 5% w/v), pH adjusting agent (q.s. to adjust pH between about 7 and about 9, optionally between about 8 and about 9), stabilizer (from about 0.01% w/v to about 1% w/v, preferably from about 0.1% w/v to about 1% w/v), surfactant (from about 0.01% w/v to about 1% w/v, preferably from about 0.01% w/v to about 0.2% w/v), preservative (from about 0.01% w/v to about 4% w/v), antioxidant (from about 0.01% w/v to about 0.5% w/v), viscosity adjusting agent (from about 0.01% w/v to about 20% w/v, preferably from about 0.1% w/v to about 5% w/v), emulsifying agent (from about 0.01% w/v to about 5% w/v), and flavoring agent (from about 0.001% w/v to about 2% w/v).

### Parenteral Formulations

In an embodiment of the present invention, the liquid pharmaceutical formulation of pitolisant is provided in a dosage form, such as solution, emulsion or suspension, suitable for parenteral administration.

In an embodiment, the present invention provides a liquid pharmaceutical formulation suitable for parenteral administration, wherein the composition comprises (i) pitolisant or a pharmaceutically acceptable salt thereof at a concentration in the range from about 0.1 mg/mL to about 400 mg/mL, and (ii) at least one pharmaceutically acceptable vehicle.

In an embodiment, the present invention provides a liquid pharmaceutical formulation suitable for parenteral administration, wherein the composition comprises (i) pitolisant or a pharmaceutically acceptable salt thereof at a concentration in the range from about 0.1 mg/mL to about 400 mg/mL, and (ii) at least one pharmaceutically acceptable excipient; and (iii) at least one pharmaceutically acceptable vehicle.

In an embodiment, the present invention provides a liquid pharmaceutical formulation suitable for parenteral administration, wherein the composition comprises (i) pitolisant or a pharmaceutically acceptable salt thereof at a concentration in the range from about 0.1 mg/mL to about 400 mg/mL, and (ii) at least one pharmaceutically acceptable excipient selected from group comprising of preservatives, pH adjusting agents, buffering agents, viscosity adjusting agents, antioxidants, tonicity adjusting agents, chelating agents, permeation enhancers, mucoadhesive agents, stabilizers, complexing agents, crystallization inhibitors, emulsifying agents, suspending agents, surfactants, anti-foaming agents, dispersing agents, and wetting agents; and (iii) at least one pharmaceutically acceptable vehicle.

In an embodiment, the present invention provides a liquid pharmaceutical formulation suitable for parenteral administration, wherein the composition comprises (i) pitolisant or a pharmaceutically acceptable salt thereof at a concentration in the range from about 0.1 mg/mL to about 400 mg/mL, and (ii) at least one pharmaceutically acceptable excipient selected from group comprising of preservatives, pH adjusting agents, buffering agents, viscosity adjusting agents, antioxidants, tonicity adjusting agents, chelating agents, permeation enhancers, mucoadhesive agents, stabilizers, complexing agents, crystallization inhibitors, emulsifying agents, suspending agents, surfactants, anti-foaming agents, dispersing agents, and wetting agents; and (iii) at least one pharmaceutically acceptable vehicle which is water.

In another embodiment of the present invention, pitolisant or a pharmaceutically acceptable salt thereof may be present at a concentration in the range from about 0.1 mg/mL to about 10 mg/mL.

In yet another embodiment of the present invention, pitolisant or a pharmaceutically acceptable salt thereof may be present at a concentration in the range from about 10 mg/mL to about 100 mg/mL.

In an embodiment of the present invention, the pitolisant is present in the formulation in the form of pitolisant hydrochloride.

In one embodiment of the invention, the formulation is an oil-based liquid depot. This formulation comprises pitolisant dissolved in a biocompatible, and biodegradable oil vehicle suitable for parenteral injection. Upon intramuscular or subcutaneous administration, this composition forms a localized depot from which the pitolisant slowly partitions into the systemic circulation. The release of the drug is thus sustained over a prolonged duration. Suitable oils for such formulations are not limited but may include sesame oil, cottonseed oil, castor oil, soybean oil, ethyl oleate, benzyl benzoate, and medium-chain triglycerides. To enhance stability and optimize performance, the oil-based formulation may further comprise one or more pharmaceutically acceptable excipients. These can include antioxidants, such as alpha-tocopherol, as well as viscosity-modifying agents or co-solvents to improve the solubility and stability of the active ingredient within the vehicle.

In another embodiment, the long-acting formulation is a microsphere-based suspension. In this configuration, pitolisant is encapsulated within a matrix of a biodegradable, biocompatible polymer to form microspheres. These microspheres are then suspended in a sterile aqueous vehicle for parenteral administration. Following injection, the polymer matrix undergoes gradual degradation in vivo, typically by hydrolysis, which results in the controlled and predictable release of the encapsulated pitolisant. The release kinetics can be precisely engineered by selecting appropriate polymers and modulating their characteristics. Suitable synthetic biodegradable polymers include, for example, poly(lactic-co-glycolic acid) (PLGA), a copolymer whose degradation rate can be tuned to achieve release over weeks to months by adjusting the lactic-to-glycolic acid ratio and molecular weight. Other synthetic polymers include poly(lactic acid) (PLA), which degrades more slowly and is suitable for release over several months, and polycaprolactone (PCL), which exhibits a very slow degradation profile ideal for formulations intended to last six months to a year or longer. Alternatively, the microspheres may be prepared using natural biodegradable polymers, which offer excellent biocompatibility. Non-limiting examples of such natural polymers include chitosan, alginate, gelatin, and collagen.

In some embodiments of the present invention, the liquid formulation of pitolisant or its pharmaceutically acceptable salt may be encapsulated in soft gelatin capsule for oral delivery for the said advantages of the liquid formulations.

In an embodiment, the present invention provides a liquid pharmaceutical formulation of pitolisant suitable for parenteral route of administration, wherein the formulation is sterilized by terminal sterilization using methods selected from the group comprising, moist heat sterilization, gamma irradiation, and electron beam sterilization, preferably moist heat sterilization.

In an embodiment, the present invention provides a liquid pharmaceutical formulation of pitolisant suitable for parenteral route of administration, wherein the formulation is sterilized by moist heat sterilization at about 110 to about 130° C. from about 2 minutes to about 30 minutes, preferably at about 121° C. for about 5 minutes to 15 minutes, preferably at about 121° C. for about 15 minutes.

In another embodiment, the present invention provides a liquid pharmaceutical formulation of pitolisant suitable for parenteral route of administration, wherein the formulation is sterilized by moist heat sterilization at about 121° C. for about 5 minutes to about 15 minutes.

In yet another embodiment, the present invention provides a liquid pharmaceutical formulation of pitolisant suitable for parenteral route of administration, wherein the formulation is sterilized by sterile filtration without use of heat. In an embodiment, the present invention provides a liquid pharmaceutical formulation of pitolisant suitable for parenteral route of administration, wherein the formulation is ready-to-dilute without requiring reconstitution at time of use prior to administration.

In another embodiment, the present invention provides a liquid pharmaceutical formulation of pitolisant suitable for parenteral route of administration, wherein the formulation is ready-to-use without requiring dilution at the time of administration prior to use.

In an embodiment, the present invention provides a liquid pharmaceutical formulation of pitolisant suitable for parenteral route of administration, wherein the formulation is sterile, preservative free, single-dose injection.

In another embodiment, the present invention provides a liquid pharmaceutical formulation of pitolisant suitable for parenteral route of administration, wherein the formulation is multiple-dose injection containing a preservative.

In an embodiment of the present invention, the liquid formulation of pitolisant suitable for parenteral administration is sterile solution comprising pitolisant hydrochloride (from about 0.01% w/v to about 20% w/v, preferably about 0.1% w/v to about 10% w/v), vehicle (q.s. to 100% w/v) and one or more optional ingredients selected from group comprising of preservative (from about 0.001% w/v to about 0.5% w/v), chelating agent (from about 0.001% w/v to about 1% w/v), pH adjusting agent (q.s. to adjust pH between about 3 and about 7, optionally between about 5 and about 7), stabilizer (from about 0.001% w/v to about 1% w/v, preferably from about 0.01% w/v to about 0.5% w/v), antioxidant (from about 0.01% w/v to about 0.5% w/v), and tonicity adjusting agent (from about 0.1% w/v to about 5% w/v).

In an embodiment of the present invention, the liquid formulation of pitolisant suitable for parenteral administration is sterile solution comprising pitolisant or a pharmaceutically acceptable salt thereof (from about 0.1% w/v to about 10% w/v), vehicle (q.s. to 100% w/v) and one or more optional ingredients selected from group comprising of preservative (from about 0.01% w/v to about 0.5% w/v), chelating agent (from about 0.01% w/v to about 1% w/v), pH adjusting agent (q.s. to adjust pH between about 5 and about 7), stabilizer (from about 0.01% w/v to about 0.5% w/v), antioxidant (from about 0.01% w/v to about 0.5% w/v), and tonicity adjusting agent (from about 0.1% w/v to about 5% w/v).

In an embodiment of the present invention, the liquid formulation of pitolisant suitable for parenteral administration is sterile, preservative free solution comprising pitolisant or a pharmaceutically acceptable salt thereof at concentration from about 0.1% w/v to about 10% w/v, optionally from about 0.1% w/v to about 5% w/v, chelating agent (from about 0.01% w/v to about 1% w/v), tonicity adjusting agent (from about 0.1% w/v to about 5% w/v), pH adjusting agent (q.s. to adjust pH between about 5 and about 7), and water for injection in amount q.s. to 100% w/v.

In an embodiment of the present invention, the liquid formulation of pitolisant suitable for parenteral administration is sterile, preservative free solution comprising pitolisant or a pharmaceutically acceptable salt thereof at concentration from about 0.1% w/v to about 10% w/v, optionally from about 0.1% w/v to about 5% w/v, pH adjusting agent (q.s. to adjust pH between about 5 and about 7), tonicity adjusting agent (from about 0.1% w/v to about 5% w/v), and water for injection in amount q.s. to 100% w/v.

In an embodiment of the present invention, the liquid formulation of pitolisant suitable for parenteral administration is sterile, preservative free solution comprising pitolisant or a pharmaceutically acceptable salt thereof at concentration from about 0.1% w/v to about 10% w/v, optionally from about 0.1% w/v to about 5% w/v, hydrochloric acid and/or sodium hydroxide (q.s. to adjust pH between about 5 and about 7), sodium chloride (from about 0.1% w/v to about 0.9% w/v), and water for injection in amount q.s. to 100% w/v.

In an embodiment of the present invention, the liquid formulation of pitolisant suitable for parenteral administration is sterile, preservative free solution comprising pitolisant or a pharmaceutically acceptable salt thereof at concentration from about 0.1% w/v to about 10% w/v, optionally from about 0.1% w/v to about 5% w/v, hydrochloric acid and/or sodium hydroxide (q.s. to adjust pH between about 5 and about 7), mannitol (from about 1% w/v to about 5% w/v), and water for injection in amount q.s. to 100% w/v.

In an embodiment of the present invention, the liquid formulation of pitolisant suitable for parenteral administration is sterile, preservative free solution comprising pitolisant or a pharmaceutically acceptable salt thereof at concentration from about 0.01% w/v to about 0.5% w/v, optionally from about 0.01% w/v to about 0.1% w/v, dilute hydrochloric acid and/or sodium hydroxide (q.s. to adjust pH between about 5 and about 7), mannitol (about 5% w/v) or sodium chloride (about 0.8% w/v), and water for injection in amount q.s. to 100% w/v.

### Emulsion (Liquid Dispersed System)

The present disclosure relates to liquid pharmaceutical formulations of pitolisant, or a pharmaceutically acceptable salt thereof, configured as dispersed lipid-based systems. These systems, which include but are not limited to emulsions, microemulsions, and self-emulsifying drug delivery systems (SEDDS), are designed to overcome challenges associated with conventional dosage forms. The core inventive concept involves a lipid or oil phase that is dispersed within an aqueous phase (or forms such a dispersion in situ). In some embodiments of the present invention, pitolisant free base is dissolved in oil phase and provided as oil-in-water emulsion. In some other embodiments, pitolisant water soluble salt such as hydrochloride salt is dissolved in aqueous phase and provided as water in oil emulsion.

A key feature of the present invention is that the pitolisant free base, which is itself an oily liquid at or near room temperature, can constitute a substantial portion or the entirety of the said lipid phase. In certain embodiments, therefore, the lipid phase may be composed solely of the pitolisant free base. In other embodiments, the lipid phase may comprise the pitolisant free base in combination with one or more pharmaceutically acceptable excipient lipids or oils (as vehicle).

By sequestering the active ingredient in this manner, the formulations are capable of providing numerous advantages, including: (i) taste masking and reduced irritation: minimizing direct contact with taste receptors and reducing gastric irritation; (ii) modulated drug release and absorption: providing for sustained release orally or controlled absorption intranasally to avoid rapid spikes of pitolisant concentration in CNS; (iii) enhanced bioavailability: improving the solubilization and subsequent absorption of pitolisant (specifically free base); (vi) improved patient compliance: offering a liquid dosage form suitable for patients with dysphagia.

In one aspect of the invention, the dispersed lipid-based system is an emulsion, a system comprising at least two immiscible liquid phases (an aqueous phase and the aforesaid lipid phase), stabilized by one or more emulsifying agents.

In another aspect, the system is microemulsion or nanoemulsion. A microemulsion or nanoemulsion provides an alternative means for achieving the benefits described herein and is formed from an aqueous phase, a surfactant, an optional co-surfactant, and the aforesaid lipid phase comprising the pitolisant free base.

In a further aspect, the formulation is a self-emulsifying drug delivery system (SEDDS). The SEDDS is an anhydrous pre-concentrate comprising the pitolisant-containing lipid phase, surfactants, and optional co-solvents, which spontaneously forms an emulsion or microemulsion upon dilution with aqueous fluids in situ.

The globule size in liquid dispersed system of the present disclosure may range from about 5 nm to about 100 micrometer, depending on drug load or oil concentration, surfactant or emulsifier used and its concentration.

In some aspects of the invention, the liquid dispersion of pitolisant or its pharmaceutical salt may be stabilized without use of emulsifying agent, wherein pitolisant itself act as emulsifier and/or surfactant and/or co-surfactant.

### Suspension

According to some aspects of the present invention, the liquid pharmaceutical formulation of pitolisant or its pharmaceutically acceptable salt is provided as suspension. Although, the free base of pitolisant exist as oily substance, it may act as solid particle at lower temperature below its freezing point or can be encapsulated in matrix wherein the drug stay in contact with liquid during its shelf-life. In some embodiments, pitolisant is in dissolved form while excipients may be provided by suspending in medium. For example, liquid filled delayed release capsule may contain pitolisant dissolved in liquid medium, while optionally suspending alkalizing agent to reduce local pH in intestine. This formulation may be provided as suspension filled in capsule such as soft gelatin or hard gelatin capsule. The term “Suspension” as used herein means a liquid with solid particles dispersed substantially throughout the formulation. The properties of a liquid suspension, according to the invention, are greatly influenced by the particle size of the drug substance, i.e. pitolisant or excipients such as alkalizer (for example, tromethamine in non-aqueous media or carbonate in aqueous media). As used herein, a “particle” may be a crystal, a granule, agglomerate, or any un-dissolved solid material. To achieve the rapid onset of activity, which is desirable, a small particle size is essential, ensuring the fastest possible dissolution of drug substance.

In some embodiments of the present invention, the suspension formulation comprises pitolisant free base at pH higher than about 5, optionally higher than about 6, optionally higher than about 7, optionally between about 5 and about 10, optionally between about 6 and about 9, optionally between about 7 and about 8.

In certain embodiments of this invention, the suspension formulation contains pitolisant free base or a corresponding salt exhibiting water solubility below approximately 10 mg/mL, and optionally below 2 mg/mL or 1 mg/mL.

In an embodiment of present invention, the particle size distribution (PSD) of Pitolisant is such that it is suitable for suspension for intranasal administration and includes at least 90% of the particles that are smaller than 10 μm, preferably from about 5 μm to about 15 μm.

### Liquid Formulation Filled Solid Dosage Forms

In certain embodiments, the stable liquid formulations of pitolisant or its pharmaceutically acceptable salts described herein are intended for oral administration encapsulated within either a liquid-filled soft gelatin capsule or a liquid-filled hard gelatin capsule. These dosage forms leverage the stability of the liquid formulation while providing alternative delivery options.

The capsules can be designed for immediate release, wherein the liquid fill is released rapidly upon disintegration of the capsule shell in the stomach. Alternatively, delayed-release liquid-filled solid formulations can be produced. This can be achieved by encapsulating the liquid formulation in standard soft or hard gelatin capsules followed by the application of an enteric (delayed-release) coating to the capsule exterior. Alternatively, specialized hard capsule shells composed of materials suitable for delayed release (e.g., DRCaps® made from HPMC, ACGcaps™ HX, or similar technologies) can be used, or specialized soft gelatin shell compositions providing enteric properties may be employed.

A key advantage of these delayed-release liquid-filled dosage forms is the potential for more rapid drug release and availability for absorption once the enteric coating dissolves or the delayed-release shell opens in the intestine, compared to conventional delayed-release tablets, pellets or multiarticulate which require the solid core or particles to disintegrate and dissolve before absorption can occur. The liquid formulation ensures the pitolisant is already in solution, potentially accelerating onset of absorption post-gastric transit.

The liquid formulation of the present invention is adaptable to a wide range of oral dosage forms by utilizing various commercially available capsule sizes, thereby enabling the creation of multiple dosage strengths suitable for diverse patient needs. In certain embodiments, the formulation is encapsulated in a liquid-filled hard gelatin capsule. The selection of hard gelatin capsule sizes may include, but is not limited to, standard sizes such as 5, 4, 3, 2, 1, 0, 00, or 000, which are capable of accommodating fill volumes ranging from approximately 0.13 mL to 1.37 mL. In alternative embodiments, the formulation is encapsulated in a soft gelatin capsule (softgel), which may be configured in various shapes, including round, oval, or oblong. For example, oval softgels may range from a size of 2 minims to 20 minims (accommodating approximately 0.12 mL to 1.23 mL), while oblong softgels may range from 4 minims to 22 minims (accommodating approximately 0.25 mL to 1.35 mL). This flexibility allows for precise dose titration and facilitates the development of formulations tailored for specific patient populations, such as pediatric or geriatric patients who benefit from smaller, easier-to-swallow capsules. Any of the aforementioned capsule sizes, whether hard or soft gelatin, may be further processed to achieve immediate or delayed release profiles, thus broadening the therapeutic utility of the pitolisant liquid formulation.

In further embodiments, the liquid fill formulation contained within the capsule (either immediate or delayed release) may additionally comprise an alkaline agent. The selection of a suitable alkaline agent depends on factors such as its solubility or suspendibility in the chosen liquid vehicle (aqueous or non-aqueous) and its compatibility with pitolisant under formulation and storage conditions. Potential alkaline agents, inspired by those explored for solid dosage forms, may include: (a) organic bases potentially offering better solubility in non-aqueous vehicles like PEG 400 or propylene glycol, such as meglumine, tromethamine, lysine, or arginine; (b) inorganic bases, which generally have low solubility in non-aqueous vehicles and may require formulation as suspensions (often using micronized material), such as magnesium carbonate, potassium bicarbonate, sodium carbonate, calcium hydroxide, calcium phosphate tribasic, magnesium oxide, potassium bicarbonate, sodium tetraborate, etc. Exemplary concentration ranges for the alkaline agent within the liquid fill could be from about 2% to about 30% by weight of the fill, aiming to achieve a desired effect such as local pH modification upon release, enhanced absorption as suggested by permeability studies.

### Pharmaceutical Excipients

### 1. Vehicles

According to some aspects, the liquid pharmaceutical composition of the present invention comprises a vehicle. The terms “vehicle”, “pharmaceutically acceptable vehicle”, “diluent” or “filler” as used herein is any ingredient or mixture or combination of ingredients, either in liquid form or solid form, that can be dissolved, homogenized or suspended, to increase the volume of the liquid composition of present invention. Non-limiting examples of vehicles are water (preferably purified water for oral or intranasal dosage form and water for injection for parenteral dosage form), sorbitol, sucrose, sugar, ethanol, mannitol, xylitol, propylene glycol, polyethylene glycol, glycerin, lactose, cellulose, starch, triglycerides, or oil. In some embodiments of the present invention, the concentration of vehicles ranges from about 5% to about 99.99%, from about 10% to about 99.99%, from about 20% to about 99.99%, from about 30% to about 99.99%, from about 40% to about 99.99%, from about 50% to about 99.99%, from about 50% to about 99.99%, from about 60% to about 99.99%, from about 70% to about 99.99%, from about 80% to about 99.99%, from about 90% to about 99.99% based on the total weight of the composition. In some embodiments, the preferred vehicles are selected from the group comprising of water, propylene glycol and polyethylene glycol.

In an embodiment of the present invention, vehicles are added in sufficient quantity (quantum satis or q.s.) to achieve the target concentration of the liquid formulation of pitolisant, after accounting for the amount of all other ingredients.

In some embodiments of the present invention, oil is used as vehicle, specifically to dissolve pitolisant free base to prepare oil-based solutions and emulsion or microemulsion.

In some embodiments of the present invention, oil is used as vehicle, specifically to dissolve pitolisant free base to prepare oil-based solutions, emulsions, or as base for liquid filled capsules. Oil used in a liquid formulation of present invention is selected from the group comprising of, without limitation, olive oil, almond oil, fish oil, avocado oil, sesame oil, canola oil, sunflower oil, castor oil, soybean oil, coconut oil, anise oil, apricot kernel oil, cinnamon oil, corn oil, cottonseed oil, rapeseed oil, mustard oil, palm oil, peanut oil, macadamia oil, beechnut oil, brazil nut oil, cashew oil, hazel nut oil, pecan oil, pine nut oil, pistachio oil, walnut oil, pumpkin seed oil, rice-bran oil, grapefruit seed oil, lemon oil, orange oil, bitter gourd oil, bottle gourd oil, buffalo gourd oil, butternut squash seed oil, edusi seed oil, watermelon seed oil, acai seed oil, black seed oil, blackcurrent seed oil, borage seed oil, evening primrose oil, flaxseed oil, amaranth oil, apricot oil, apple seed oil, argan oil, babassu oil, ben oil, borneo tallow nut oil, cape chestnut oil, Citrus sinensis flower oil, clove oil, coriander oil, eucalyptus oil, lanolin oil, mineral oil, nutmeg oil, peppermint oil, spearmint oil, vegetable oil, animal oil, hydrogenated vegetable oil, polyoxyl vegetable oil, apricot kernel oil PEG-6 esters, Citrus sinensis flower oil, palm kernel oil glycerides (hydrogenated), corn oil mono- and di-glycerides (c18), hydrogenated castor oil, hydrogenated coconut oil, hydrogenated cottonseed oil, hydrogenated palm oil, hydrogenated soybean oil, lanolin alcohol-mineral oil, lavender oil, light mineral oil, lime oil, natural oil, orange oil terpeneless, PEG-40 castor oil, PEG-60 hydrogenated castor oil, pine needle oil (Pinus sylvestris), polyoxyl 35 castor oil, polyoxyl 40 hydrogenated castor oil, sassafras oil, liquid triglyceride of 6 to 12 carbon chain length, thyme oil, turpentine oil, hydrogenated vegetable oil glyceride, Miglyol® 818, Miglyol® 812, Miglyol® 840, Labrafac®, Captax® 385, Capmul®, Imwitor® 988, Imvitor® 948, oleic acid, linoleic acid, Maisine® 35-1, Capryol® 90, Maisine® CC, Peceol™ or combination thereof. The concentration of oil in the disclosed liquid formulation of pitolisant ranges from about 1% to about 99.99%, optionally from about 30% to about 99.99% based on the total weight of the composition depending on concentration of pitolisant in the formulation and formulation type. For example, microemulsion of pitolisant free base with concentration of about 1 mg/mL may need lower concentration of oil than oil based oral solution.

### 2. Preservatives

According to some aspects, the liquid pharmaceutical composition of the present invention comprises one or more preservative(s). The term “preservative” used herein refers to any ingredient or mixture or combination of ingredients added to prevent or inhibit the growth of microorganisms, thereby ensuring the safety and shelf-life of the product. Preservatives are especially important in multi-dose preparations, where repeated access can introduce contaminants, and their inclusion helps maintain the sterility and integrity of the formulation throughout its intended use. In an embodiment, the pharmaceutical composition of the liquid formulation of pitolisant comprises one or more preservative(s) selected from the group comprising of benzoic acid and the sodium or potassium salts thereof, ethanol, isopropanol, methanol, butyl alcohol, benzalkonium chloride, benzyl alcohol, benzethonium chloride, butylparaben, cetylpyridinium chloride, chlorobutanol, chlorocresol, cresol, dehydroacetic acid, ethylparaben, ethylparaben sodium, methylparaben, methylparaben sodium, phenol, phenylethyl alcohol, phenylmercuric acetate, phenylmercuric nitrate, potassium benzoate, potassium sorbate, propylparaben, propylparaben sodium, sodium dehydroacetate, sodium propionate, sorbic acid, thimerosal, thymol, or combinations thereof.

In some embodiments of the present invention, the concentration of preservatives ranges from 0.001% to about 10%, preferably from about 0.01% to about 5%, preferably from about 0.01% about 2%, preferably from about 0.01% to about 1%, preferably from about 0.01% to about 0.5% based on the total weight of the composition.

In some other embodiments of the present invention, the concentration of preservatives ranges from 0.1% to about 10%, preferably from about 0.1% to about 5%, preferably from about 0.1% about 2%, preferably from about 0.1% to about 1%, preferably from about 0.1% to about 0.5% based on the total weight of the composition.

In some embodiments, liquid formulation of the present invention may contain an ingredient or a group of ingredients at concentration that is sufficient to prevent microbial growth by generating higher osmotic pressure. Non-limiting examples for such ingredients are sugars (sorbitol, mannitol, xylitol, dextrose, sucrose, lactose, etc.) at concentration from about 5% to about 90%, preferably from about 10% to about 70%, preferably from about 40%; and salts (such as sodium chloride, potassium chloride, etc.) at concentration from about 0.5% to about 10%, preferably from about 1% to about 5%. In some other embodiments, pitolisant or its pharmaceutically acceptable salt may be present at a concentration that prevents microbial growth and act as self-preservative and it is still within the scope of the present invention. In yet other embodiments of the present invention, non-aqueous solvent used as vehicle, such as polyethylene glycol, propylene glycol, glycerin, ethanol or combination thereof at quantity enough (for example from about 5-99%, preferably from about 50-99%, preferably more than about 50% of the total weight of the composition) preventing microbial growth acting as preservative and it is still within the scope of the invention.

### 3. Sweeteners

According to some aspects, the liquid pharmaceutical composition of the present invention comprises one or more sweetening agent(s). The terms “sweetening agent” or “sweetener” refers to both bulk (caloric) and intense (non-caloric) sweeteners, which impart a sweet taste to the preparation. Non-limiting examples of sweeteners include glucose, sucralose, neotame, aspartame, advantame, trehalose, fructose, xylose, dextrose, galactose, tagatose, maltose, sucrose, glycerol, dulcitol, mannitol, lactitol, sorbitol, xylitol, saccharine or the corresponding sodium, potassium or calcium salt, cyclamate or the corresponding sodium or calcium salt, acesulfame or the potassium salt thereof, dulcin or ammonium glycyrrhizinate (Magnasweet® 110 or similar), alitame, inulin, steviol glycoside, isomalt, neohesperidin dihydrochalcone, thaumatin and the like or any combinations thereof.

In some embodiments of the present invention, the concentration of sweeteners ranges from about 0.001% to about 90%, preferably from about 0.001% to about 40%, preferably from about 0.01% to about 30%, preferably from about 0.01% to about 20%, preferably from about 0.01% to about 10%, preferably from about 0.01% to about 5%, preferably from about 0.01% to about 2%, preferably from about 0.1% to about 2% based on the total weight of the composition.

In some other embodiments of the present invention, the concentration of sweeteners ranges from about 0.01% to about 70%, preferably from about 0.1% to about 40%, preferably from about 1% to about 40%, preferably from about 5% to about 40%, preferably from about 10% to about 40%, preferably from about 20% to about 40%, preferably from about 30% to about 40%, preferably from about 10% to about 20%, preferably from about 20% to about 30%, preferably from about 5% to about 15% based on the total weight of the composition. In some embodiments of the present invention, the intense sweetener may require less quantity for taste masking (for example sweetener like aspartame, neotame, advantame, sucralose, saccharin, etc.) while low-intensity sweetener (such as glucose, sorbitol, xylitol, mannitol, etc.) may require larger quantity of sweetener for taste masking.

### 4. Flavors

According to some aspects, the liquid pharmaceutical composition of the present invention comprises one or more flavoring agent(s). The terms “flavoring agent,” or “flavor” as used herein, refer to an agent or a mixture of agents that adds flavor to a mixture. A flavoring agent is selected from the group comprising of a natural flavor, an artificial flavor, and mixtures thereof. Non-limiting examples of flavoring agents are vanilla, Citrus oil, including lemon, orange, grape, lime and grapefruit, and fruit essences, including apple, banana, pear, peach, strawberry, raspberry, cherry, plums pineapple, apricot, peppermint, tutti frutti flavor and so forth and the like or any combinations thereof. Solid forms, such as spray dried forms of flavoring agents, may also be useful in the liquid dosage forms disclosed herein.

In some embodiments of the present invention, the concentration of flavoring agent ranges from about 0.001% to about 5%, preferably from about 0.001% to about 2%, preferably from about 0.001% to about 1%, preferably from about 0.001% to about 0.1%, preferably from about 0.01% to about 0.1% based on total weight of the composition.

In some other embodiments of the present invention, the concentration of flavoring agent ranges from about 0.01% to about 5%, preferably from about 0.01% to about 5%, preferably from about 0.1% to about 5%, preferably from about 1% to about 5%, preferably from about 2% to about 5% based on total weight of the composition.

### 5. pH Adjusting Agents

According to some aspects, the liquid pharmaceutical composition comprises one or more pH adjusting agent(s). As used herein, the term “pH adjusting agent” refers to a component or combination of components sufficient to adjust a pharmaceutical composition's pH. According to some aspects, the pH adjusting agent is sufficient to raise or lower the pharmaceutical composition's initial pH to a pharmaceutically acceptable range (i.e., not toxic or producing unacceptable side effects). Additionally, or alternatively, the pH adjusting agent may be sufficient to maintain the pharmaceutical composition's pH in a pharmaceutically acceptable range over a certain period of shelf life. Example of pH adjusting agents includes without limitation, acetic acid, adipic acid, ammonia, ascorbic acid, barium hydroxide, benzoic acid, calcium hydroxide, carbonic acid, cesium hydroxide, citric acid, formic acid, fumaric acid, gluconic acid, glutamic acid, hydrochloric acid, isothionic acid, lactic acid, lithium hydroxide, malic acid, maleic acid, mandelic acid, methane sulfonic acid, monopotassium phosphate, monosodium phosphate, mucic acid, nitric acid, oxalic acid, p-toluenesulfonic acid, pantothenic acid, phosphoric acid, phthalic acid, potassium hydroxide, pyrophosphoric acid, rubidium hydroxide, sodium hydroxide, strontium hydroxide, succinic acid, sulfuric acid, tartaric acid, or combination thereof. In one example, the pharmaceutical composition comprises hydrochloric acid, sodium hydroxide, or a combination thereof.

In some embodiments of the present invention, the concentration of pH adjusting agent ranges up to about 10%, preferably up to about 8%, preferably up to about 5%, preferably up to about 2% based on total weight of the composition. In some embodiments of the present invention, pH adjusting agent (such as hydrochloric acid) may be used in mole ratio of pitolisant such as from about 0.5 to about 1.5:1.0 (pH adjusting agent:pitolisant), optionally from about 0.9 to 1.1:1.0, optionally from about 1.0 to 1.1:1.0, optionally from about 1.00 to 1.05:1.00, optionally ≥1.00:1.00.

In some other embodiments of present invention, pH adjusting agent donating more than one proton or accepting more than one anion (such as sulfuric acid) may have reduced ratio by dividing provided mole above by number of donating anions.

In some other embodiments of the present invention, the concentration of pH adjusting agent may depends on the pH of formulation and quantity to be used is generally considered as “quantity sufficient (q.s.) to target pH”.

According to some aspects, each of the pH adjusting agents may together or independently have a concentration in the range from about 0.0001 N to about 20 N, preferably from about 0.01 N to about 1 N.

According to some aspects, the pharmaceutical composition may be free of pH adjusting agents as described herein. It should be understood that the pH-adjusting agent-free pharmaceutical composition may have an initial pH within a pharmaceutically acceptable pH range, as described herein and/or may have a pH in a pharmaceutically acceptable range over a certain period of shelf life, as described herein.

### 6. Buffering Agents

According to some aspects, the liquid pharmaceutical composition of the present invention comprises one or more buffering agent(s). The term “buffering agent”, also known as buffer system, refers to ingredients that helps maintaining the pH of formulation. Examples of buffering agents include, without limitation, salts of citric acid, tartaric acid, ascorbic acid, phosphoric acid, acetic acid, carbonic acid, phthalic acid, succinic acid, glutamic acid, benzoic acid, gluconic acid, formic acid, fumaric acid, lactic acid, isothionic acid, malic acid, maleic acid, mandelic acid, nitric acid, mucic acid, oxalic acid, pantothenic acid, p-toluenesulfonic acid, methane sulfonic acid, sulfuric acid, hydrochloric acid, or combination thereof. Additionally, pharmaceutically acceptable amino acids such as methionine, cysteine, glycine, etc. can also be used as buffering agents.

In some other embodiments of the present invention, the concentration of buffering agent ranges from 0.001% to 10%, preferably from about 0.001% to about 5%, preferably from about 0.001% to about 2%, preferably about 0.001% to about 1%, preferably about 0.001% to 0.5%, preferably about 0.001% to about 0.25% based on the total weight of the composition.

In some other embodiments of the present invention, the concentration of buffering agent ranges from 0.01% to 10%, preferably from about 0.01% to about 5%, preferably from about 0.01% to about 2%, preferably about 0.01% to about 1%, preferably about 0.01% to 0.5%, preferably about 0.01% to about 0.25% based on the total weight of the composition.

In some other embodiments of the present invention, the concentration of buffering agent ranges from 0.1% to 10%, preferably from about 0.1% to about 5%, preferably from about 0.1% to about 2%, preferably about 0.1% to about 1%, preferably about 0.1% to 0.5%, preferably about 0.1% to about 0.25% based on the total weight of the composition.

It is understood that a higher quantity of acid or base can be used in higher than range mentioned in the disclosure considering that they neutralize the effect of each other and are still within the scope of the invention. In some embodiments of the present invention, the concentration of pH adjusting agent, or buffering agent is net quantity after neutralization by counter ions.

### 7. Viscosity Adjusting Agents

According to some aspects, the liquid pharmaceutical composition of the present invention comprises one or more viscosity adjusting agent. As used herein, the term “viscosity adjusting agent” refers to any compound or combination of compounds added to a formulation of the present invention to modify or maintain its viscosity, ensuring optimal flow, pourability, stability, and ease of administration of the final liquid formulation.

A viscosity-adjusting agent can be utilized to achieve the desired formulation consistency based on the specific requirements of the end user. For instance, individuals with Prader-Willi Syndrome present hypotonia resulting in residual dysphagia or subclinical dysphagia. In such cases, oral liquids with lower viscosity increase the risk of aspiration into the lungs, making higher viscosity liquids more appropriate. In another example, a modest increase in the viscosity of nasal spray formulations can extend their residence time on the nasal mucosa, potentially facilitating more thorough absorption.

In one embodiment, viscus non-aqueous solvent such as propylene glycol, glycerin, polyethylene glycol, tocopherol or vitamin E (such as α-tocopherol) may be used to increases or adjust the viscosity for desired properties. In another embodiment, alcohol (such as ethanol) may be used to reduce the viscosity of the formulation. In yet another embodiment, combination of high and low viscosity solvent may be combined with making aqueous or non-aqueous liquid formulation of the present invention with desired viscosity and/or other characteristics.

In certain embodiments, achieving bioequivalence between an oral liquid and a solid dosage form, such as a tablet, requires modulating the absorption rate of pitolisant. As a highly soluble salt, pitolisant hydrochloride is prone to rapid absorption from an oral solution, which can result in a Cₘₐₓ that significantly exceeds that of the reference tablet. To mitigate this pharmacokinetic mismatch, a viscosity-adjusting agent may be incorporated into the liquid formulation of the present invention. This agent increases the viscosity of the vehicle, thereby slowing gastric emptying and reducing the drug's diffusion rate to the gastrointestinal mucosa. This controlled slowing of absorption ensures the Cₘₐₓ of the liquid formulation falls within the bioequivalent range of the tablet.

According to some aspects, the liquid pharmaceutical composition of the present invention comprises a viscosity-adjusting agent from the class of cellulose derivatives. The term “cellulose derivative” refers to semi-synthetic polymers derived from natural cellulose that hydrate and swell in water to form a viscous, three-dimensional polymer network, thereby controlling the formulation's flow properties and the drug's rate of absorption. Examples include, without limitation, Hydroxypropyl Methylcellulose (HPMC), Sodium Carboxymethylcellulose (Sodium CMC), Hydroxyethyl Cellulose (HEC), Methylcellulose, or combinations thereof. In certain embodiments, the concentration of such cellulose derivatives may range from about 0.001% w/v to about 10.0% w/v, depending on the nature of specific agent and desired viscosity.

According to some aspects, the liquid pharmaceutical composition of the present invention comprises a viscosity-adjusting agent from the class of natural gums. The term “natural gum” refers to high molecular weight polysaccharides derived from natural sources, such as microbial fermentation or botanical origins, which are efficient at increasing viscosity at low concentrations and may impart specific rheological properties such as pseudoplasticity. Examples include, without limitation, Xanthan Gum, Guar Gum, Acacia, Tragacanth, Carrageenan, or combinations thereof. In certain embodiments, the concentration of such natural gums may range from about 0.001% to about 2.0% w/v, optionally from about 0.001% to about 0.5% w/v, with agents like xanthan gum often being effective at the lower end of this range.

According to some aspects, the liquid pharmaceutical composition of the present invention comprises a viscosity-adjusting agent from the class of synthetic polymers. The term “synthetic polymer” refers to man-made polymers engineered with specific properties to modify the rheology of the formulation, often providing high viscosity with unique characteristics such as pH-dependent or temperature-dependent thickening. Examples include, without limitation, Carbomers, Polyvinylpyrrolidone (PVP), Poloxamers, Polyvinyl Alcohol (PVA), or combinations thereof. In certain embodiments, the concentration of such synthetic polymers varies by type; for example, high-efficiency agents like carbomers may be used from about 0.05% to about 1.0% w/v, whereas agents like polyvinylpyrrolidone may be used in a range from about 1.0% to about 10.0% w/v.

In some embodiments of the present invention, the concentration of viscosity adjusting agent ranges from about 0.001% to about 10%, from about 0.01% to about 5%, from about 0.1% to about 2%, from about 0.1% to about 1% by total weight of the composition, depending on the desired consistency and properties of final formulation. The quantity of viscosity adjusting agents used in this invention may vary depending on the specific properties and type of agent selected. For instance, a viscous liquid pitolisant formulation of present invention utilizing 1% w/v hypromellose K4M can also be produced with hypromellose K15M or K100M to achieve similar viscosity; however, the required quantity of hypromellose K15M or K100M will be substantially lower than that used in the K4M-based formulation.

### 8. Antioxidants

In some embodiments of the present invention, the liquid pharmaceutical composition of the present invention comprises one or more antioxidants. The term “antioxidant” refers to agents that protect any ingredients of the composition from oxidative degradation. Examples include, without limitation, methionine, sodium/potassium bisulfite, sodium metabisulfite, potassium metabisulfite, sodium ascorbate, ascorbic acid, butylated hydroxytoluene, butylated hydroxyanisole, cysteine, glutathione, monothioglycerol, propyl gallate, sodium/potassium sulfite, tocopherol (such as alpha-tocopherol) or its salt and esters (such as D-alpha-tocopheryl polyethylene glycol 1000 succinate), or combination thereof. It is understood that some of the antioxidants, as disclosed in the examples of the present disclosure, may surprisingly degrade pitolisant in specific environment and/or at certain concentration and should be used carefully.

In some embodiments of the present invention, the concentration of antioxidants ranges from about 0.001% to about 5%, optionally from about 0.01% to about 2%, optionally from about 0.01% to about 1%, optionally from about 0.01% to about 0.5%, optionally from about 0.01% to about 0.1% based on total weight of the composition.

In some embodiments of the present invention, the concentration of antioxidants ranges from about 0.05% to about 2%, optionally from about 0.1% to about 2%, optionally from about 0.1% to about 2%, optionally from about 0.1% to about 1%, optionally from about 0.1% to about 0.5% based on total weight of the composition.

### 9. Tonicity Adjusting Agents

In some embodiments of the present invention, the liquid pharmaceutical composition of the present invention comprises one or more tonicity adjusting agent. As used herein, the term “tonicity adjusting agent” refers to any ingredient or combination of ingredients added to the pharmaceutical formulation to adjust or contributing to its osmotic pressure, thereby ensuring that the formulation is isotonic or near-isotonic with physiological fluids, such as nasal mucosa, when administered intranasally, or to a soft tissue or muscle when administered by intramuscular route, or into the blood when administered intravenously. Tonicity adjusting agents contribute to patient comfort, prevent irritation, and help maintain the stability and efficacy of the formulation upon administration. Non-limiting examples of tonicity adjusting agents that may be used in the pharmaceutical compositions of the present invention include alanine, arginine, betaine, boric acid, calcium chloride (CaCl₂)), calcium lactate, dextran, dextrose, fructose, glycerin, glycine, glucose, histidine, hydroxyethyl starch, lactose, lysine, magnesium chloride, magnesium sulfate (MgSO₄), maltose, mannitol, polyethylene glycol (PEG), potassium chloride (KCl), potassium citrate, proline, propylene glycol, raffinose, ribose, sodium acetate, sodium benzoate, sodium bicarbonate (NaHCO₃), sodium citrate, sodium chloride (NaCl), sodium gluconate, sodium lactate, sodium phosphate (NaH₂PO₄ or NazHPO₄), sodium sulfate (Na₂SO₄), sodium tartrate, sodium thiosulfate (Na₂S₂O₃), sorbitol, sucrose, taurine, trehalose, tromethamine (Tris), xylitol, etc., as well as mixtures thereof. In some embodiments, the concentration of tonicity adjusting agents ranges from about 0.1% to about 10%, from about 0.2% to about 8%, from about 0.5% to about 5% by weight based on the total composition, with the precise amount selected to achieve the desired osmolality, for example, from about 100 mOsmol/Kg to about 1000 mOsmol/Kg, preferably from about 250 mOsmol/kg to about 350 mOsmol/kg. The specific selection, combination, and concentration of tonicity adjusting agents is tailored according to the overall composition and intended route of administration to ensure optimal therapeutic performance and patient tolerability. In some embodiments of the present invention, pitolisant hydrochloride in dissolved form also contributes to the osmolality of the final formulation.

At given concentration of pitolisant hydrochloride in water or other vehicle, it will also act as tonicity adjusting agent to achieve target osmolality and it is within the scope of present invention. In one embodiment, the present invention provides pitolisant liquid formulation suitable for intranasal administration comprising of pitolisant hydrochloride at concentration of about 50 mg/mL, that may have osmolality of about 300 mOsmol/Kg, which is isotonic to bodily fluid and may not need to add additional tonicity adjusting agent. In this example, pitolisant hydrochloride act as tonicity adjusting agent.

In some embodiments, the present invention provides a pitolisant liquid formulation intended for intranasal and/or parenteral administration, comprising pitolisant hydrochloride or alternative pitolisant salts at a concentration that achieves patient-tolerable tonicity without causing significant discomfort and without need of additional tonicity adjusting agent.

### 10. Chelating Agents

In some embodiments, the liquid pharmaceutical composition of the present invention comprises one or more chelating agents. The term “chelating agent” refers to compounds that helps to stabilize the formulation by means of chelating undesired compounds or elements or ions. Suitable chelating agents which may be used in the present invention include, but are not limited to, edetate disodium (EDTA); edetate trisodium, edetate tetrasodium, edetate calcium disodium; and diethylene amine pentaacetate or derivatives thereof. In certain preferable embodiments, the formulations comprise disodium edetate. In some embodiments, any hydrate or anhydrous or polymorphs of the said chelating agents are within the scope of the invention.

In some other embodiments, hydrocarboxylic acids such as citric acid, amino acids such as histidine, siderophore such as deferoxamine, may also be used as chelating agents in the liquid formulation of the present invention.

In some embodiments of the present invention, the concentration of chelating agents ranges from 0.001% to 5%, preferably from about 0.001% to about 2%, preferably from about 0.001% to about 1%, preferably from about 0.001% to about 0.5%, preferably from about 0.001% to about 0.1% based on the total weight of the composition.

In some other embodiments of the present invention, the concentration of chelating agents ranges from 0.01% to 5%, preferably from about 0.01% to about 2%, preferably from about 0.01% to about 1%, preferably from about 0.01% to about 0.5%, preferably from about 0.1% to about 0.2% based on the total weight of the composition.

### 11. Permeability Enhancing Agents

According to some aspects, the liquid pharmaceutical composition of the present invention comprises one or more permeability enhancers. As used herein, the terms “permeation enhancer” or “permeability enhancer” refers to an ingredient or group of ingredients that are incorporated into the liquid formulation of the present invention to increase the permeability of the pitolisant across biological membranes. Permeation enhancers function by temporarily modifying the barrier properties of the membrane, facilitating improved absorption or bioavailability of the drug. Non-limiting examples of permeation enhancers include, without limitation: alcohol, arachidonic acid, benzethonium chloride, benzethonium bromide, benzalkonium chloride, capric acid, caproic acid, carvone, cetylpyridium chloride, chitosans, citric acid, 6-cyclohexyl-1-hexyl-P-D-maltopyranoside, n-decyl-P-D-maltopyranoside, dimethyl sulfoxide, dodecyl dimethyl aminopropionate, 1-O-n-Dodecyl-P-D-maltopyranoside, dodecylpolyethyleneglycolether, edetate disodium dihydrate, enanthic acid, glyceryl monooleate, glyceryl monostearate, glycofurol, isopropyl myristate, isopropyl palmitate, pelargonic acid, lanolin, lauric acid, light mineral oil, limonene, linoleic acid, lysine, menthol, myristic acid, myristyl alcohol, oleic acid, oleyl alcohol, palmitic acid, peppermint oil, polyoxyethylene alkyl ethers, polyoxylglycerides, polysorbates, pyrrolidone, sodium caprate, sodium desoxycholate, sodium deoxyglycolate, sodium glycocholate, sodium hydroxybenzoyal amino caprylate, sodium lauryl sulfate, sodium taurocholate, stearic acid, thymol, tricaprylin, triolein, undecylenic acid, and combinations thereof.

In some embodiments of the present invention, the concentration of permeation enhancer ranges from about 0.001% to about 10%, preferably from about 0.001% to about 5%, preferably from about 0.001% to about 2%, or preferably from about 0.001% to about 1% based on the total weight of the composition. The precise concentration may be adjusted depending on desired permeability enhancement, physical properties of the formulation, and compatibility with other ingredients.

### 12. Mucoadhesive Agents

According to some aspects, the liquid pharmaceutical composition of the present invention comprises one or more mucoadhesive agents. As used herein, the term “mucoadhesive agent” refers to an ingredient or group of ingredients that enhance the adhesion of the formulation to mucosal surfaces, thereby prolonging the residence time and improving the efficacy of the active pharmaceutical ingredient of the present invention. Mucoadhesive agents function by forming physical or chemical interactions with the mucin layer covering mucosal tissues. Examples of mucoadhesive agents include, without limitation, natural polymers such as chitosan, alginate, hyaluronic acid, gelatin, tragacanth, xanthan gum, guar gum, and pectin; synthetic and semi-synthetic polymers such as carbomers (Carbopol) of all grades (such as Carbopol 934, Carbopol 940, Carbopol 971P, and others), polyvinyl alcohol (including all pharmaceutical grades), polyvinylpyrrolidone (such as K12, K30, and K90 grades), polyethylene oxide (including Sentry Polyox grades such as WSR N10, N80, N750, and N205), polyacrylic acid (all USP and NF grades), hydroxypropyl methylcellulose (HPMC grades such as E3, E5, K4M, K15M, and K100M), methylcellulose (USP and food grades), carboxymethyl cellulose (including sodium carboxymethyl cellulose grades like low, medium, and high viscosity types), and poloxamers (including grades such as Poloxamer 188 and 407); as well as cellulose derivatives (all grades), sodium alginate, and acacia gum. In some embodiments of the present invention, the concentration of mucoadhesive agent ranges from about 0.001% to about 10%, preferably from about 0.01% to about 5%, preferably from about 0.1% to about 2%, preferably from about 0.1% to about 1%, and more preferably from about 0.1% to about 0.5% based on the total weight of the composition. The precise concentration may be adjusted depending on desired mucoadhesive strength, physical properties of the formulation, and compatibility with other ingredients.

### 13. Stabilizers

According to some aspects, the liquid pharmaceutical composition of the present invention comprises one or more stability enhancing agent or stabilizer. The terms “stability enhancing agent” or “stabilizer” are used herein to inhibit, prevent, slow down, or reduce the degradation of pitolisant. More specifically, stability-enhancing agents include amino acids such as alanine, glycine, glutamate, sodium glutamate, L-arginine, lysine, L-cysteine or methionine; sodium chloride or sodium sulfate salts; sodium hydroxide, potassium hydroxide, calcium hydroxide, lactic acid, glycolic acid, succinic acid, pyruvic acid, citric acid, ethylenediaminetetraacetic acid (EDTA), non-transitioning metal ions such as zinc, magnesium and calcium or mixtures thereof; natural or synthetic gums, cellulosic derivatives such as carboxy methyl cellulose, ethyl cellulose, hydroxyethyl cellulose, hydroxyl propyl cellulose, hydroxyl propyl methylcellulose, methylcellulose, polyanionic cellulose; cyclodextrins; sugars; sugar alcohols, mannitol, glycerol or glycerin, sorbitol; monosaccharides, disaccharides or polysaccharides or combinations thereof.

In some embodiments of the present invention, the concentration of the stabilizer ranges from 0.001% to 20%, preferably from about 0.001% to about 10%, preferably from about 0.01% to about 5%, preferably from about 0.1% to about 1%, preferably from about 0.15% to 0.5%, preferably from about 0.15% to 0.25% based on the total weight of the composition. In some embodiments, vehicle may act as stabilizer and in such cases stabilizer could be used from about 10% to about 99.99% w/w.

In some other embodiments of the present invention, the concentration of the stabilizer ranges from 0.1% to 20%, preferably from about 1% to about 20%, preferably from about 1% to about 10%, preferably from about 1% to about 5% based on the total weight of the composition.

### 14. Complexing Agents

According to some aspects, the liquid pharmaceutical composition of the present invention comprises one or more complexing agent(s). The term “complexing agent,” as used herein, refers to a compound or mixture of compounds that interacts with an active pharmaceutical ingredient to form a stable complex, thereby enhancing solubility, stability, palatability (taste masking) or bioavailability within the formulation. In the context of pitolisant liquid pharmaceutical compositions, suitable complexing agents may include, but are not limited to, Cyclodextrins (such as α-cyclodextrin, β-cyclodextrin, γ-cyclodextrin, and their derivatives like hydroxypropyl-β-cyclodextrin, sulfobutyl ether-β-cyclodextrin), ethylenediaminetetraacetic acid (EDTA), citric acid, tartaric acid, or other pharmaceutically acceptable agents capable of forming inclusion or coordination complexes. The concentration of complexing agents in compositions of present invention typically ranges from about 0.01% to about 70% by weight of the total formulation, optionally from about 1% to about 40%, optionally from about 1% to about 20%, depending on the nature and binding affinity of the selected agent, and the desired solubility and/or stability and/or taste masking properties. In some embodiments of the present invention, the complexing agent may be used in a mole ratio from about 1:1 to about 15:1 (complexing agent:pitolisant), preferably from about 1:1 to about 5:1, optionally from about 1:1 to about 2:1, optionally about 1:1. In some other embodiments of the present invention, the mole ratio of complexing agent:pitolisant is <1:1, for example, about 0.5:1 to about 1:1, about 0.8:1 to about 1:1. In some embodiments of the present invention, when complexing agent is used, pitolisant is used as free base or its poorly soluble salt to achieve concentration higher than saturated concentration in absence of complexing agent. In some embodiments, primary purpose of complexation is to mask the taste or improve solubility or both. In some embodiments, salts with higher aqueous solubility may be used with complexing agent to achieve the said benefits of the complexing agents.

### 15. Crystallization Inhibitors

According to some aspects, the liquid pharmaceutical composition of the present invention comprises one or more crystallization inhibitor(s). As used herein, the term “crystallization inhibitors” refers to ingredients that prevent or reduce the formation and/or growth of crystals and/or amorphous particles of pitolisant or its pharmaceutically acceptable salt within a liquid formulation, thereby stabilizing the active ingredient in a supersaturated or amorphous state and improving the clarity and homogeneity of the final product. Suitable crystallization inhibitors include, without limitation, hydrophilic polymers such as polyvinylpyrrolidone (PVP, including PVP K12, PVP K15, PVP K17, PVP K25, PVP K30, PVP K60, PVP K90, or PVP K120), polyethylene glycol (PEG 200, PEG 300, PEG 400, PEG 600, or PEG 1000), hydroxypropyl methylcellulose (HPMC), hydroxypropyl cellulose, methylcellulose, sodium carboxymethyl cellulose, polyvinyl alcohol, poloxamers, povidone, copovidone, cyclodextrins, and polysorbates (such as Polysorbate 20, Polysorbate 40, Polysorbate 60, or Polysorbate 80). In some embodiments of the present invention, the concentration of crystallization inhibitor ranges from about 0.01% to about 10% based on the total weight of the composition.

### 16. Suspending Agents

According to some aspects, the liquid pharmaceutical composition of the present invention comprises one or more suspending agent(s). As used herein, the term “suspending agent” refers to a substance that helps keep the solid particle dispersed and prevent or slow down the settling to the bottom of the liquid. Suspending agents suitable for use in the liquid oral suspensions of the present invention include, cellulose derivatives such as methylcellulose, sodium carboxymethylcellulose, hydroxypropyl methyl cellulose, polyvinyl pyrrolidone (PVP, such as PVP K12, PVP K15, PVP K17, PVP K25, PVP K30, PVP K60, PVP K90, or PVP K120), alginate, guar gum, xanthan gum, carrageenan, acacia gum, tragacanth, chitosan, dextran, gelatin, polyethylene glycol, polyoxyethylene and polyoxypropylene ether.

As used herein, the term “polyhydric alcohol” refers to a compound with more than one hydroxyl group. Non-limiting examples of polyhydric alcohols that can be used are glycerin, propylene glycol, polyethylene glycol, xylitol, maltitol, sorbitol, sucrose, sucralose, glucose, and mannitol.

In some embodiments of the present invention, the concentration of polyhydric alcohol ranges from about 0.1% to 75%, preferably from about 0.1% to about 40%, preferably from about 0.1% to about 5%, preferably from about 0.1% to about 1%, preferably from about 0.1% to 0.5% based on the total weight of the composition.

In an embodiment of the present invention, the concentration of suspending agent ranges from about 0.01% to 5%, preferably from about 0.05% to about 5%, preferably from about 0.1% to about 4%, preferably from about 0.1% to about 3%, preferably from about 0.1% to about 2% based on the total weight of the composition.

### 17. Surfactants

According to some aspects, the liquid pharmaceutical composition of the present invention comprises one or more surfactant(s). As used herein, the term “surfactant” refers to any ingredient that reduces the surface tension between particle and liquid medium, allowing for better wetting and preventing agglomeration in the liquid formulation of the present invention.

In an embodiment, the liquid formulation of pitolisant may contain suitable surfactants such as ionic or non-ionic surface-active agents. Suitable ionic surfactants include, but are not limited to, those containing carboxylate, sulfonate, and sulfate ions. Examples of anionic surfactants include potassium, sodium, ammonium salts of long chain alkyl sulfonates, and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate; quaternary ammonium compounds such as benzalkonium chloride, benzethonium chloride, cetrimonium bromide, stearyl dimethylbenzyl ammonium chloride, polyoxyethylene. In an embodiment, the concentration of ionic surfactant ranges from about 0.01% to 3%, preferably from about 0.05% to about 2%, preferably from about 0.05% to about 1%, preferably from about 0.05% to about 0.5%, preferably from about 0.05% to about 0.25% based on the total weight of the composition.

Suitable nonionic surfactants optionally used include, but are not limited to, glycol stearates such as ethylene glycol monostearate, propylene glycol myristate, glyceryl monostearate, glyceryl stearate, polyglyceryl-4-oleate, sorbitan acylate, sucrose acylate, polyethylene glycol dilaurate, polyethylene glycol monolaurate, polysorbates, polyoxyethylene octylphenylether, polyethylene glycol cetyl ether, polyoxyethylene tridecyl ether, polypropylene glycol butyl ether, stearoyl monoisopropanolamide, polyoxyethylene hydrogenated tallow amide, polyoxyl-ethylated castor oils (CREMOPHOR®), polyoxyethylene esters of 12-hydroxystearic acid (SOLUTOL®) and PEGylated glycerides (LABRASOL®). In another embodiment, the concentration of non-ionic surfactant ranges from about 0.02% to 2%, based on the total weight of the composition. In another embodiment, polysorbate is the non-ionic surfactant at a concentration of less than about 7% (w/w) of the total composition, less than about 6% (w/w) of the total composition, less than about 5% (w/w) of the total composition, less than about 4% (w/w) of the total composition, less than 3% (w/w) of the total composition, less than 2% (w/w) of the total composition, less than 1% (w/w) of the total composition.

### 18. Anti-Foaming Agents

According to some aspects, the liquid pharmaceutical composition of the present invention comprises one or more anti-foaming agent(s). As used herein, the term “anti-foaming agent” or “defoamers” refers to ingredients suitable to be used in the formulation of the present invention that disrupt the stability of the foam or prevent the foaming by destabilizing the thin liquid films or lamellae that form the bubble walls, causing them to rupture and collapse. Non-limiting examples of anti-foaming agents are simethicone, polydimethylsiloxane, organomodified silicones, mineral oil, vegetable oil, polypropylene glycol, fatty alcohols, aliphatic esters, triglycerides or combination thereof. In an embodiment of the present invention, the concentration of anti-foaming agent is from about 0.001% w/v to about 5% w/v, optionally from about 0.001% w/v to about 1% w/v, optionally from about 0.01% w/v to about 0.5% w/v.

### 19. Dispersing Agents

According to some aspects, the liquid pharmaceutical composition of the present invention comprises one or more dispersing agent(s). As used herein, the term “dispersing agent” refers to any substance that facilitates the uniform distribution of solid particles within a liquid medium, thereby preventing aggregation or clumping and ensuring homogeneity of the formulation of the present invention. Dispersing agents improve the stability and consistency of suspensions by enhancing the separation of individual particles and maintaining their even distribution throughout storage and dosing. Non-limiting examples of dispersing agents include polysorbates (PS, such as PS 20, PS 40, PS 60, PS 80), lecithin, polyvinylpyrrolidone (PVP, such as PVP K12, PVP K30, PVP K90), poloxamers, sodium lauryl sulfate, sodium citrate, sodium carboxymethyl cellulose, and phospholipids, as well as certain surfactants and amphiphilic polymers. In some embodiments of the present invention, the concentration of dispersing agent ranges from about 0.01% to about 5%, preferably from about 0.05% to about 2%.

### 20. Wetting Agents

According to some aspects, the liquid pharmaceutical composition of the present invention comprises one or more wetting agent(s). As used herein, the term “wetting agent” refers to any substance that facilitates the dispersion and penetration of solid particles by reducing the interfacial tension between the solid and the liquid medium in a formulation. Wetting agents play a crucial role in enhancing the ability of the liquid to spread across the surface of solids, thereby improving uniformity and consistency in pharmaceutical suspensions and other liquid formulations. Non-limiting examples of wetting agents suitable for use in the present invention include, but are not limited to, polysorbates (such as polysorbate 20, Polysorbate 40, Polysorbate 60, or Polysorbate 80), sodium lauryl sulfate, poloxamers, lecithin, polyoxyethylene sorbitan monolaurate, polyvinylpyrrolidone (PVP), sorbitan esters, cetyl alcohol, stearyl alcohol, glycerol monostearate, polyethylene glycol (such as PEG 200, 300, 400, 600, or 1000), and sodium dioctyl sulfosuccinate. In some embodiments of the present invention, the concentration of wetting agent ranges from about 0.01% to about 5% based on the total weight of the composition, preferably from about 0.05% to about 2%, and more preferably from about 0.1% to about 1%.

### 21. Emulsifying Agents

In certain embodiments, the present invention provides a liquid pharmaceutical composition comprising one or more emulsifying agents. As used herein, the term “emulsifying agent” refers to any substance capable of facilitating the formation and stabilization of emulsions by reducing interfacial tension between immiscible phases, such as oil and water. Reduction of interfacial tension enables uniform dispersion of one liquid phase within another, thereby preventing phase separation and maintaining homogeneity throughout the shelf life and administration of the composition.

The emulsifying agent may be selected from natural, semi-synthetic, or synthetic compounds. Non-limiting examples of suitable emulsifying agents include:
(a) Phospholipids, such as lecithin (egg lecithin, soy lecithin), phosphatidylcholine, phosphatidylethanolamine, phosphatidylserine, phosphatidylinositol, and hydrogenated lecithin;
(b) Polysorbates, including polysorbate 20, polysorbate 40, polysorbate 60, and polysorbate 80;
(c) Sorbitan esters, such as sorbitan monostearate, sorbitan monooleate, sorbitan monopalmitate, sorbitan tristearate, and sorbitan trioleate;
(d) Polyoxyethylene derivatives, including polyoxyethylene sorbitan monolaurate, polyoxyethylene sorbitan monostearate, and polyoxyethylene castor oil derivatives such as CREMOPHOR® EL and CREMOPHOR® RH40;
(e) Polyethylene glycol (PEG) esters and PEGylated glycerides, including PEG 300, PEG 400, PEG 600, LABRASOL®, and GELUCIRE® series;
(f) Poloxamers, such as Poloxamer 188, Poloxamer 338, and Poloxamer 407;
(g) Fatty alcohols, including cetyl alcohol, stearyl alcohol, and oleyl alcohol;
(h) Polyvinylpyrrolidone (PVP), such as PVP K12, PVP K30 and PVP K90;
(i) Monoglycerides and diglycerides, including glyceryl monostearate, glyceryl monooleate, and glyceryl dioleate;
(j) Amphiphilic polymers, such as hydroxypropyl methylcellulose (HPMC), hydroxypropyl cellulose (HPC), polyvinyl alcohol, and polyethylene oxide derivatives;
(k) Ionic surfactants, including sodium lauryl sulfate, sodium stearate, and benzalkonium chloride; and
(l) Other non-limiting examples such as PEG stearates, PEG oleates, PEG palmitates, PEG hydrogenated castor oil derivatives, PEG-40 hydrogenated castor oil, and PEG-60 hydrogenated castor oil.

In some embodiments, the emulsifying agent is present in the liquid pharmaceutical composition of the present invention in an amount ranging from about 0.001% to about 15% by weight of the total composition. In certain embodiments, the amount ranges from about 0.01% to about 10%, optionally from about 0.05% to about 5%, and in preferred embodiments from about 0.1% to about 2%. For formulations containing a high proportion of oil phase, the concentration of emulsifying agent may be increased up to about 20% to achieve desired stability.

The concentration of emulsifying agent may be adjusted based on formulation parameters, including the proportion of oil phase, the type of emulsifier employed, and the target globule size of the emulsion. For example, low-oil formulations (oil phase less than about 10%) may utilize emulsifier concentrations between about 0.01% and about 1%; medium-oil formulations (oil phase between about 10% and about 30%) may utilize emulsifier concentrations between about 0.5% and about 5%; and high-oil formulations (oil phase greater than about 30%) may utilize emulsifier concentrations between about 2% and about 10%, or higher for specialized systems. Optimization of these parameters ensures physical stability and performance, maintaining uniformity and preventing phase separation throughout the intended shelf life of the composition.

### 22. Film Forming Agents

Film forming agents suitable for use in liquid-filled solid dosage forms, such as soft gelatin or hard gelatin capsules, include a variety of polymers that provide structural integrity, encapsulation efficiency, and controlled release properties. For capsule shells, commonly used film formers are gelatin (from bovine or porcine sources), hydroxypropyl methylcellulose (HPMC), pullulan, and starch derivatives. Plasticizers such as glycerin, sorbitol, or polyethylene glycol may be incorporated to impart desired flexibility and reduce brittleness. For capsule coatings, additional film formers may include polyvinyl alcohol, ethylcellulose, methacrylic acid copolymers (such as Eudragit® types), and shellac. These agents can be selected and combined based on the required dissolution profile, stability, and compatibility with the fill formulation. Functional coatings may further include enteric or sustained-release polymers to modulate drug release in the gastrointestinal tract.

In certain embodiments, the capsule material may be modified with coloring agents, opacifiers (such as titanium dioxide), or flavors to enhance product appearance and patient acceptability.

In embodiments requiring delayed release, the liquid filled solid dosage form is coated with one or more enteric polymers selected for their pH-dependent solubility characteristics. Suitable enteric coating polymers for use in such formulations include, but are not limited to, cellulose-based derivatives such as cellulose acetate phthalate (CAP), hydroxypropyl methylcellulose phthalate (HPMCP), hydroxypropyl methylcellulose acetate succinate (HPMCAS), cellulose acetate trimellitate (CAT), and certain derivatives of carboxymethylcellulose coating polymers. Other suitable polymers include vinyl polymers, for instance, polyvinyl acetate phthalate (PVAP), and a range of acrylic copolymers. The acrylic copolymers may comprise various methacrylic acid copolymers, including those commercially available under the Eudragit® trade name (e.g., Eudragit® L 100-55, Eudragit® L, Eudragit® S, Eudragit® FS), as well as poly(methacrylic acid-co-methyl methacrylate). Furthermore, naturally derived polymers may be employed for the enteric coating, such as the purified resin shellac, zein, and polysaccharides including sodium alginate, pectin, guar gum, and chitosan. These polymers may be utilized individually or as combinations thereof. The selection of the specific polymer or polymer blend is based on its ability to substantially resist dissolution in the acidic gastric environment while dissolving at a desired pH within the intestine to enable targeted drug release, thereby protecting acid-sensitive drugs, preventing gastric irritation, and improving therapeutic outcomes. The quantity of delayed release polymers may range from about 1% w/w to about 50% w/w, optionally from about 5% w/w to about 25% w/w.

### Method of Preparation

The present invention also provides a method of preparing a stable liquid pharmaceutical formulation, the said method comprising the steps of: (i) solubilizing pitolisant or its pharmaceutically acceptable salt in a vehicle; (ii) solubilizing one or more optional pharmaceutically acceptable excipients; (iii) optionally filtering the formulated liquid; (iv) filling the filtered liquid into a primary packaging container; and (v) sealing the container. In this process, the pitolisant free base is dissolved in aqueous acidic medium to form pitolisant hydrochloride in situ, followed by incorporation of excipients selected from solubilizers, sweeteners, buffering agents, pH adjusters, preservatives, antioxidants, chelating agents, surfactants, stability-enhancing agents, crystallization inhibitors, or viscosity adjusting agents, then filling and sealing with optional inert gas overlay.

In an embodiment, the present invention provides a process for preparing pitolisant solution suitable for oral administration comprising the steps of: (1) adding vehicle to manufacturing vessel; (2) adding one or more optional ingredients one-by-one with mixing until dissolved; (3) adding pitolisant or a pharmaceutically acceptable salt thereof with mixing until dissolved; (4) optionally filtering the solution using suitable filter such as form about 0.2 μm to about 20 μm pore size; (5) filling the pitolisant solution in suitable primary packaging containers; and (6) closing the containers. Such containers may be further packaged in carton, optionally with delivery devices such as syringe or measuring cup or measuring spoon.

In an embodiment, the present invention provides a process for preparing pitolisant solution suitable for oral/buccal or sublingual administration in the form of unit dose or bi-dose spray comprising the steps of: (1) adding vehicle to manufacturing vessel; (2) adding one or more optional ingredients one-by-one with mixing until dissolved; (3) adding pitolisant or a pharmaceutically acceptable salt thereof with mixing until dissolved; (4) optionally filtering the solution using suitable filter such as about 0.2 μm or about 0.45 μm filter; (5) filling the pitolisant solution in vials, closed with polymeric stopper and assemble with unit or bi-dose spray device; and (6) optionally packaging such device further into tray or carton. In some embodiments, the formulations may be preservative free, wherein the containers are sterilized by terminal sterilization using suitable methods such as moist heat sterilization at about 121° C. for about 5 to about 15 minutes.

In an embodiment, the present invention provides a process for preparing pitolisant solution suitable for oral/buccal or sublingual administration in the form of multiple dose spray comprising the steps of: (1) adding vehicle to manufacturing vessel; (2) adding one or more optional ingredients one-by-one with mixing until dissolved; (3) adding pitolisant or a pharmaceutically acceptable salt thereof with mixing until dissolved; (4) optionally filtering the solution using suitable filter such as about 0.2 μm or about 0.45 μm filter; (5) filling the pitolisant solution in containers and closing with closure assembled with metering spray device; and (6) optionally packaging such device further into tray or carton.

In an embodiment, the present invention provides a process for preparing pitolisant solution suitable for intranasal administration in the form of unit dose or bi-dose spray comprising the steps of: (1) adding vehicle to manufacturing vessel; (2) adding one or more optional ingredients one-by-one with mixing until dissolved; (3) adding pitolisant or a pharmaceutically acceptable salt thereof with mixing until dissolved; (4) optionally adjusting pH between about 4 and about 6; (5) optionally filtering the solution using suitable filter such as 0.2 μm filter; (6) filling the pitolisant solution in vials, closed with polymeric stopper and assemble with unit or bi-dose nasal spray device; and (7) optionally packaging such device further into tray or carton. In some embodiments, the formulations may be preservative free, wherein the containers are sterilized by terminal sterilization using suitable methods such as moist heat sterilization at about 121° C. for about 5 to about 15 minutes.

In an embodiment, the present invention provides a process for preparing pitolisant solution suitable for intranasal administration in the form of multiple dose spray comprising the steps of: (1) adding vehicle to manufacturing vessel; (2) adding one or more optional ingredients one-by-one with mixing until dissolved; (3) adding pitolisant or a pharmaceutically acceptable salt thereof with mixing until dissolved; (4) optionally adjusting pH between about 4 and about 6; (5) optionally filtering the solution using suitable filter such as 0.2 μm filter; (6) filling the pitolisant solution in containers and closing with closure assembled with metering spray device; and (7) optionally packaging such device further into tray or carton. In some embodiments of the present invention, the product is sterilized by aseptic filtration without terminal sterilization.

In an embodiment, the present invention provides a process for preparing pitolisant solution suitable for injectable route of administration comprising the steps of: (1) adding vehicle to manufacturing vessel; (2) adding optional ingredients one-by-one with mixing until dissolved; (3) adding pitolisant or a pharmaceutically acceptable salt thereof with mixing until dissolved; (4) sterilizing the solution using sterile grade 0.2 μm filter; (5) aseptically filling the pitolisant solution in suitable primary package such as cartridge, vial or pre-fillable syringe; (6) closing the primary container with appropriate closure; and (7) optionally terminally sterilizing the containers using suitable methods such as moist heat sterilization at about 121° C. for about 5 to about 20 minutes.

In an embodiment, the present invention provides a process for preparing pitolisant suspension suitable for oral spray (oral or buccal or sublingual route) or oral liquid comprising the steps of: (1) adding vehicle to manufacturing vessel; (2) adding one or more ingredients one-by-one with mixing until dissolved or suspended; (3) suspending pitolisant free base or its salt with mixing until homogenous suspension obtained; (4) filling the pitolisant suspension in suitable containers; and (5) closing the containers. Such containers may be further packaged in carton, optionally with delivery devices such as syringe or measuring cup or measuring spoon.

In an embodiment, the present invention provides a process for preparing pitolisant suspension suitable for intranasal administration comprising the steps of: (1) adding vehicle to manufacturing vessel; (2) adding one or more ingredients one-by-one with mixing until dissolved or suspended; (3) suspending pitolisant free base or its salt with mixing until homogenous suspension obtained; (4) filling the pitolisant suspension in containers and closing with closure assembled with metering spray device; and (5) optionally packaging such device further into tray or carton.

In an embodiment, the present invention provides a process for preparing pitolisant suspension suitable for oral spray (oral or buccal or sublingual route) or oral liquid comprising the steps of: (1) adding vehicle to manufacturing vessel; (2) adding one or more ingredients one-by-one with mixing until dissolved or suspended; (3) dissolving pitolisant hydrochloride with mixing until homogenous solution obtained; (4) adjusting pH between about 7 and about 9 wherein the pitolisant precipitates and forms homogenous suspension; (5) filling the pitolisant suspension in suitable containers; and (6) closing the containers. Such containers may be further packaged in carton, optionally with delivery devices such as syringe or measuring cup or measuring spoon.

In an embodiment, the present invention provides a process for preparing pitolisant suspension suitable for intranasal administration comprising the steps of: (1) adding vehicle to manufacturing vessel; (2) adding one or more ingredients one-by-one with mixing until dissolved or suspended; (3) dissolving pitolisant hydrochloride with mixing until homogenous solution obtained; (4) adjusting pH between about 7 and about 9 wherein the pitolisant precipitates and forms homogenous suspension; (5) filling the pitolisant suspension in containers and closing with closure assembled with metering spray device; and (6) optionally packaging such device further into tray or carton.

In an embodiment, the present invention provides a process for preparing a pitolisant solution suitable for oral, intranasal, or injectable administration by utilizing a complexing agent. The process comprises the steps of: (1) adding a vehicle, such as purified water, to a manufacturing vessel; (2) adding a complexing agent, such as hydroxypropyl-β-cyclodextrin (HPBCD) or Sulfo-butylether-β-cyclodextrin (SBE-β-CD), with mixing until fully dissolved; (3) adding the pitolisant salt, such as pitolisant hydrochloride, with mixing until a clear solution is obtained; (4) adding an alkalizing agent, such as sodium hydroxide, to adjust the pH to a range where the pitolisant salt converts to its undissociated free base form, which then forms an inclusion complex with the dissolved complexing agent; (5) optionally filtering the resulting solution using a suitable sterile filter; (6) filling the solution into primary packaging containers, such as vials or spray devices; and (7) closing the containers. This process leverages the cage-like structure of cyclodextrins to encapsulate the less soluble pitolisant free base, thereby increasing its solubility and/or stability and/or mask the taste in the formulation.

In an embodiment, the present invention provides a process for preparing a pitolisant emulsion suitable for oral, or intranasal, administration. The process comprises the steps of: (1) adding a vehicle, such as purified water, to a manufacturing vessel; (2) adding pitolisant hydrochloride and mixing until fully dissolved; (3) adding alkalizer or base or pH adjusting agent (to adjust pH between about 7 to about 9) to partially or fully neutralize pitolisant salt by converting into pitolisant free base with continuous mixing and/or homogenization; (4) adding one or more ingredients selected from the group comprising of preservatives, sweeteners, flavors, pH adjusting agents, buffering agents, viscosity adjusting agents, antioxidants, tonicity adjusting agents, chelating agents, permeation enhancers, mucoadhesive agents, stabilizers, complexing agents, crystallization inhibitors, suspending agents, surfactants, anti-foaming agents, dispersing agents, and wetting agents; (5) optionally filtering the emulsion using a suitable filter; (6) filling the emulsion into primary packaging containers, such as bottles (oral liquid) or spray devices (oral or intranasal spray); and (7) closing the containers.

In an embodiment, the present invention provides a process for preparing a pitolisant emulsion suitable for oral, or intranasal, administration. The process comprises the steps of: (1) preparing aqueous phase by dissolving one or more water soluble ingredients; (2) preparing oil phase by dissolving pitolisant free base and one or more oil soluble ingredients; (3) adding oil phase into water phase by continuous mixing and/or homogenizing until homogeneous emulsion is obtained; (4) optionally filtering the emulsion using a suitable filter; (5) filling the emulsion into primary packaging containers, such as bottles (oral liquid) or spray devices (oral or intranasal spray); and (6) closing the containers.

In some versions of this invention, a liquid pitolisant formulation is made by forming the salt directly during the manufacturing process (in-situ salt formation) using an acid (such as hydrochloric acid, succinic acid, tartaric acid, citric acid, oxalic acid, etc, preferably hydrochloric acid). For this, pitolisant free base is suspended in suitable vehicle such as water at or below room temperature (such as 15-25° C. or 2-8° C.), a dilute acid solution is slowly added in about equal mole ratio (from about 0.5:1 to about 1:1; acid:pitolisant) with mixing to obtain a clear solution of pitolisant salt, followed by adding one or more optional excipients and making up the volume to batch size, filtering the solution and filling into containers, and closing the containers. In some aspect, order of addition can be changed to achieve same results such as preparing solution of vehicle and acid (dilute acidic solution) and then adding pitolisant free base and mixing until complete dissolution. End point of the salt conversion can be determined by complete dissolution and/or achieving target pH of the solution (such as achieving pH between about 2 and about 6) and/or using fixed quantity of acid (such as equal mole ratio) added and mixing until clear solution obtained. The concentration of dilute acid solution may be about 0.001 N hydrochloric acid or about 0.01 N hydrochloric acid or about 0.1 N hydrochloric acid or about 1N hydrochloric acid or about 2 N Hydrochloric acid. The concentration of acid depends on the final concentration of pitolisant hydrochloride salt. For example, 0.01N hydrochloric acid may be suitable for liquid formulation of pitolisant at concentration of about 10 mg/mL, similarly, about 0.1 N hydrochloric acid may be suitable for formulation of pitolisant at concentration of about 100 mg/mL. The in-situ salt formulation and/or method of preparation of such salt formulation of present invention can be used in any of the solution formulations described in the present disclosure.

In an embodiment, the present invention provides a process for preparing a delayed release soft gelatin capsule containing pitolisant. The method comprises the steps of: (1) preparing a pitolisant-containing fill formulation by dissolving or suspending pitolisant free base or a pharmaceutically acceptable salt thereof in a suitable vehicle such as oils (e.g., medium chain triglycerides, sesame oil) or polyethylene glycols, optionally with excipients such as surfactants, suspending agents, or stabilizers to achieve the desired viscosity and homogeneity; (2) incorporating enteric coating agents or pH-sensitive polymers (such as acrylic polymers, e.g. Eudragit® L or S, hydroxypropyl methylcellulose phthalate, or cellulose acetate phthalate) into the fill or as a separate layer to ensure delayed release properties in gastric conditions; (3) encapsulating the prepared fill formulation into soft gelatin capsules using conventional encapsulation techniques; (4) optionally drying the capsules under controlled temperature and humidity to achieve the desired shell integrity; and (5) optionally applying an additional enteric coating to the capsule shell if required for enhanced delayed release performance. The resulting soft gelatin capsules provide delayed release of pitolisant in the gastrointestinal tract, protecting the active ingredient from degradation in the stomach and promoting release in the intestine.

### Packaging

In an embodiment, the pitolisant liquid formulation of the present invention is filled into a suitable pharmaceutically acceptable container selected from the group comprising of bottles, bags, sprayers, ampules (plastic or glass), blisters, sachets, syringes, cartridges, and vials.

In another embodiment, the pitolisant liquid formulation of the present invention is filled into a pharmaceutically acceptable container suitable for single or multiple-dose delivery for oral, intranasal or injectable administration.

In an embodiment, the pitolisant liquid formulation of the present invention is filled into a suitable pharmaceutically acceptable container that reasonably protects the composition from air and light that otherwise may degrade pitolisant or any vehicle or excipient within the container.

In an embodiment, the pitolisant liquid formulation of the present invention is administered via oral route to the patient as a fluid stream, drop(s), droplet(s), spray, or combination thereof.

In an embodiment, the pharmaceutically acceptable container for the pitolisant liquid formulation is a bottle, wherein the bottle is selected from a group comprising of glass bottles or plastic bottles, wherein glass bottle is selected from a group consisting of Type I, II, and III borosilicate glass bottles, wherein the glass bottle may be amber color glass bottle or clear glass bottle.

In another embodiment, the pharmaceutically acceptable container for the pitolisant liquid formulations is a bottle, wherein the bottle is selected from a group comprising of high-density polyethylene (HDPE) bottle, polyethylene terephthalate (PET) and polypropylene (PP), wherein the plastic bottle may be amber color, white opaque or translucent plastic bottle or any other color.

In an embodiment, the ready-to-use liquid formulation of pitolisant is packaged in flexible polymeric bag contained in secondary outer packaging.

In some embodiments, the glass or polymeric containers for the liquid pitolisant formulations may be provided with a fill volume in the range from about 0.05 mL to about 500 mL, optionally from about 1 mL to about 250 mL, optionally from about 1 mL to about 10 mL, optionally from about 10 mL to about 50 mL, optionally from about 50 mL to about 100 mL, optionally from about 100 mL to about 150 mL, optionally from about 150 mL to about 200 mL, optionally from about 200 mL to about 250 mL, optionally from about 250 mL to about 300 mL, optionally from about 300 mL to about 500 mL. In some other embodiments, the fill volume of pitolisant liquid formulation is about 1 mL, about 2 mL, about 3 mL, about 5 mL, about 10 mL, about 12 mL, about 15 mL, about 20 mL, about 25 mL, about 30 mL, about 40 mL, about 50 mL, about 60 mL, about 80 mL, about 90 mL, about 100 mL, about 115 mL, about 125 mL, about 150 mL, about 180 mL, about 200 mL, about 210 mL, about 240 mL, about 250 mL, about 270 mL, about 300 mL, about 320 mL, about 350 mL, about 400 mL, about 450 mL or up to about 500 mL fill volumes. In some other embodiments, the volume filled in primary container is about 0.5 ounce, about 1 ounce, about 2 ounce, about 3 ounce, about 4 ounce, about 5 ounce, about 6 ounce, about 7 ounce, about 8 ounce, about 9 ounce, about 10 ounce, about 11 ounce, about 12 ounce, about 14 ounce, about 15 ounce, or up to about 16 ounce. Any volume within this range is within the scope of the present invention.

In some embodiments of the present invention, the liquid formulation filled solid dosage forms may be provided in glass or plastic bottles with a capacity or size of about 1 ounce to about 20 ounce, about 2 ounce, about 3 ounce, about 4 ounce, about 5 ounce, about 6 ounce, about 7 ounce, about 8 ounce, about 9 ounce, about 10 ounce, about 11 ounce, about 12 ounce, about 14 ounce, about 15 ounce, or up to about 20 ounce.

In some embodiments of the present invention, the liquid formulation filled solid dosage forms may be provided in glass or plastic bottles in pack of about 10 count, about 15 count, about 20 count, about 25 count, about 30 count, about 35 count, about 40 count, about 45 count, about 50 count, about 60 count, about 70 count, about 80 count, about 90 count, about 100 count, about 120 count, about, 150 count, about 200 count, about 250 count, or up to about 300 count per bottle.

In some embodiments of the present invention, the liquid formulation filled solid dosage forms may be provided in blister pack (such as aluminum-aluminum, or aluminum-plastic or paper) in pack size of about 10 count, about 15 count, about 20 count, about 25 count, about 30 count, about 35 count, about 40 count, about 45 count, about 50 count, about 60 count, about 70 count, about 80 count, about 90 count, about 100 count, about 120 count, about, 150 count, about 200 count, about 250 count, or up to about 300 count per blister.

In some embodiments, the liquid formulation filled solid dosage forms may be filled in containers along with desiccant or oxygen scavengers to protect from moisture and oxygen.

In an embodiment, the pharmaceutical composition of the present invention may be further packaged as a convenience kit comprising a bottle with a child-resistant cap, adapter or flow restrictor, and dosing syringe.

In another embodiment, the liquid letermovir formulations may be packaged in a metered device that protects the composition from air, light, and microbial contamination during storage and use.

In an embodiment of the present invention, the liquid formulation of pitolisant or its pharmaceutically acceptable salt is administered to patients using syringe and delivery tube, such as nasogastric tube, gastrostomy tube, jejunostomy tube, or gastrojejunal tube.

In some embodiments of the present invention, the pitolisant liquid formulation is filled in USP type I glass container and placed in unit dose nasal spray devices similar to that disclosed in U.S. Pat. No. 6,708,846 or similar devices suitable for nasal administration in the form of a spray. This nasal spray can be packaged in blister tray and placed in carton that protects the product from light.

In some embodiments of the present invention, the liquid formulation of pitolisant or its pharmaceutically acceptable salt may be filled and packaged into unit-dose, bi-dose or multiple dose nasal spray devices manufactured by Aptar Pharma, Nemera, Bona Pharma or other manufacturers and currently available or approved in the market. In some embodiments, the pitolisant liquid formulation of the present invention are filled and assembled into spray devices with advanced characteristics such as preservative free pump, springless pump, recyclable pump, etc.

### Formulation Characteristics

In some embodiments of the present invention, the pH of the pitolisant liquid formulation of the present invention is between about 1 and about 7, optionally between about 2 and about 6.5, optionally between about 2 and about 6, optionally between about 2 and about 5.5, optionally between about 2 and about 5, optionally between about 3 and about 5, optionally between about 3 and about 6, optionally between 3 and about 6.5, optionally between about 4 and about 6.5, optionally between about 5 and 6.5, optionally between about 5.5 and 6.5, optionally between about 3.5 and about 6.5, optionally between about 3.5 and about 5.5, optionally about 2.5, optionally about 3, optionally about 3.5, optionally about 4.0, optionally about 4.5, or optionally about 5.0, optionally about 5.5, optionally about 6, optionally about 6.5, optionally less than about 7, optionally less than about 6.5, optionally less than about 5. In one example, pH of the formulation is from about 3 to about 5. In another example, the pH of the formulation of present invention is about 5 to about 6.5. In some embodiments of the present invention, the pitolisant solution formulation suitable oral administration may have pH between about 2 and about 7. In some other embodiments of the present invention, the pitolisant suspension or emulsion formulation suitable oral administration may have pH higher than about 7, or between about 7 and about 9. In some embodiments of the present invention, the pitolisant solution formulation suitable intranasal administration may have pH between about 4 and about 6.

In some embodiments, the pitolisant liquid formulation of the present invention has water like viscosity such as about 1 cP. In some other embodiments, the viscosity of the formulation is higher than 1 cP, such as about 1 to 3 cP, about 3 to 10 cP, about 10 to 20 cP, about 20 to 30 cP, about 30 to 50 cP, about 50 to 100 cP, about 100 to about 1000 cP, about 1000 to about 1750 cP, about 1500 to about 3000 cP, or about 3000 to about 5000. In some other embodiments, the viscosity of the formulation is from about 1 to 50 cP, preferably from about 5 to 50 cP. In yet other embodiments, the viscosity of the formulation is from about 10 to 500 cP, preferably from about 50 to 250 cP. Higher than water viscosity may help increase residence time of the pitolisant formulation on nasal mucosa for better absorption. Additionally, viscous liquid formulation of the present invention may ease the administration in patients with subclinical dysphagia (such as PWS). The viscosity range provided here is when tested using capillary viscometer, such as U-tube Ostwald capillary viscometer with ID of about 0.5 mm or about 1 mm, using water as reference. Viscosity of the pitolisant liquid formulation of the present invention may depend on the route of administration and end use needs. For instance, injectable formulations of the present invention may have water like viscosity for intravenous use, while sustained release formulation suitable for depot injection may have viscosity like gel (such as from about 3000 cP to about 100,000 cP). Oral solution may have water like viscosity or even higher up to about 5000 cP for slower drug release to match the Cₘₐₓ with tablet. Nasal spray formulation may have slightly higher viscosity from about 5 cP to about 100 cP, optionally from about 5 cP to about 50 cP for higher residence time on nasal mucosa. Viscosity of pitolisant liquid formulation of present invention to be used for PWS patients may have viscosity from about 10 cP to about 2000 cP, optionally from about 50 to about 100 cP, optionally from about 100 to about 400 cP, optionally from about 400 to 800 cP to avoid aspiration, depending on patients' condition and needs.

In some embodiments of the present invention, the liquid formulation of pitolisant suitable for oral administration has viscosity of which an IDDSI (International Dysphagia Diet Standardization Initiative) level is classified in a range of 0-3, optionally 1-3, optionally 2 when viscosity is measured using Framework Flow Test.

In some embodiments, the pitolisant liquid formulation of the present invention has osmolality from about 100 mOsmol/kg to about 2000 mOsmol/kg, from about 200 mOsmol/Kg to about 1000 mOsmol/Kg, from about 250 mOsmol/Kg to about 800 mOsmol/Kg, from about 250 mOsmol/Kg to about 550 mOsmol/Kg, from about 250 mOsmol/Kg to about 350 mOsmol/Kg. In yet other embodiments, the osmolality of the pitolisant liquid formulation is about 280 to 320 mOsmol/Kg. In one example, the osmolality of the pitolisant liquid formulation is about 270 to about 330 mOsmol/Kg.

In some embodiments of the present invention, the nasal spray delivers, upon actuation, pitolisant formulation in the form of spray, wherein less than 10% (D10) of the droplets have a size smaller than about 10 μm, optionally smaller than about 5 μm, and/or at least 50% (D50) of the droplets have a size from about 10 μm to 150 μm, preferably from about 40 μm to about 80 μm, and/or at least 90% (D90) of the droplet have a size smaller than 300 μm, preferably smaller than 150 μm, more preferably smaller than 120 μm.

In some embodiments of the present invention, the nasal spray delivers, upon actuation, pitolisant formulation in the form of spray, wherein plume angle of the spray is from about 25° to about 75°, preferably from about 30° to about 60°.

In some embodiments of the present invention, at least 90% of the droplets in the dispersed phase of emulsion formulations are in the range from about 0.5 μm to about 100 μm.

In some embodiments of the present invention, at least 90% of the droplets in the dispersed phase of microemulsion formulations are in the range from about 10 nm to about 500 nm.

In some embodiments, the particle size distribution (PSD) of pitolisant in pharmaceutical suspension formulation of the present invention comprises at least 10% of particles smaller than about 30 microns, and/or at least 50% of particles smaller than about 70 microns, and/or at least 90% of particles smaller than about 150 microns.

In some embodiments of the present invention, the particle size distribution (PSD) of pitolisant in pharmaceutical suspension formulation of the present invention comprises at least 10% of particles smaller than about 10 microns, and/or at least 50% of particles smaller than about 50 microns, and/or at least 90% of particles smaller than about 100 microns.

In some embodiments of the present invention, the particle size distribution (PSD) of pitolisant in pharmaceutical suspension formulation of the present invention comprises at least 10% of particles smaller than about 10 microns, and/or at least 50% of particles smaller than about 25 microns, and/or at least 90% of particles smaller than about 50 microns.

In some embodiments of the present invention, the particle size distribution (PSD) of pitolisant in pharmaceutical suspension formulation of the present invention comprises at least 10% of particles smaller than about 2 microns, and/or at least 50% of particles smaller than about 5 microns, and/or at least 90% of particles smaller than about 10 microns.

In some embodiments of the present invention, the particle size of pitolisant in suspension formulation comprises at least 90% of particles smaller than about 300 microns, preferably smaller than about 200 microns, more preferably smaller than about 100 microns, more preferably smaller than about 90 microns, more preferably smaller than about 80 microns, more preferably smaller than about 70 microns, more preferably smaller than about 60 microns, more preferably smaller than about 50 microns, more preferably smaller than about 45 microns, more preferably smaller than about 40 microns, more preferably smaller than about 35 microns, more preferably smaller than about 33 microns, more preferably smaller than about 30 microns, more preferably smaller than about 28 microns, more preferably smaller than about 25 microns, more preferably smaller than about 23 microns, more preferably smaller than about 20 microns, more preferably smaller than about 18 microns, more preferably smaller than about 16 microns, more preferably smaller than about 15 microns, more preferably smaller than about 14 microns, more preferably smaller than about 13 microns, more preferably smaller than about 12 microns, more preferably smaller than about 11 microns, more preferably smaller than about 10 microns, more preferably smaller than about 9 microns, more preferably smaller than about 8 microns, more preferably smaller than about 7 microns, more preferably smaller than about 6 microns, or more preferably smaller than about 5 microns. In some other embodiments, the particle size of pitolisant in suspension formulation comprises at least 90% of particles are within the range from about 5 microns to about 10 microns.

In some embodiments of the present invention, the pitolisant solution suitable for intranasal administration is filled in unit-dose or bi-dose or multiple dose nasal spray device which forms plume of droplets when actuated at force between about 5N and about 50N, optionally between about 10N and about 50N.

In some embodiments of the present invention, the liquid pharmaceutical composition is encapsulated within delayed release capsules such as soft gelatin or hard gelatin capsule. Without wishing to be bound by any theoretical principles, the liquid filled-soft gelatin capsule may dissolve faster than delayed release dosage form with solid core such as enteric coated tablet, enteric coated pellets, enteric coated tablets or pellets or particulates filled in immediate release capsule or similar delayed release formulations with solid core. In some embodiments, the dissolution time of the delayed release capsule in phosphate buffer pH 6.8 is less than about 60 minutes, optionally less than about 30 minutes, optionally less than about 25 minutes, optionally less than about 15 minutes, optionally less than about 10 minutes, optionally less than about 5 minutes, optionally between about 2 to 5 minutes, optionally between about 5 to 10 minutes, optionally between about 10 to 30 minutes when tested using appropriate dissolution method given in pharmacopeia such as USP.

### Stability and Impurity Embodiments

In some embodiments of the present invention, the liquid pharmaceutical formulation of pitolisant is stable for at least 1 week, preferably for at least 2 weeks, preferably for at least 3 weeks, preferably for at least about 1 month, preferably for at least about 2 months, preferably for at least about 3 months, preferably for at least about 4 months, preferably for at least about 5 months, preferably for at least about 6 months, preferably for at least about 8 months, preferably for at least about 10 months, preferably for at least about 12 months, preferably for at least about 15 months, preferably for at least about 18 months, preferably for at least about 21 months, preferably for at least about 24 months, preferably for at least about 27 months, preferably for at least about 30 months, preferably for at least about 33 months, preferably for at least about 36 months at room temperature (about 15-25° C.).

According to some aspects, the pharmaceutical composition of the present invention may have an initial total impurity concentration of no more than about 5%, more preferably no more than about 4.5%, more preferably no more than about 4%, more preferably no more than about 3.5%, more preferably no more than about 3%, more preferably no more than about 2.5%, more preferably no more than about 2%, more preferably no more than about 1.5%, more preferably no more than about 1%, and most preferably no more than about 0.5%.

According to some aspects, the pharmaceutical composition of the present invention may have an initial total impurity concentration of no more than about 1%, more preferably no more than about 0.99%, more preferably no more than about 0.98%, more preferably no more than about 0.97%, more preferably no more than about 0.96%, more preferably no more than about 0.95%, more preferably no more than about 0.94%, more preferably no more than about 0.93%, more preferably no more than about 0.92%, more preferably no more than about 0.91%, more preferably no more than about 0.90%, more preferably no more than about 0.89%, more preferably no more than about 0.88%, more preferably no more than about 0.87%, more preferably no more than about 0.86%, more preferably no more than about 0.85%, more preferably no more than about 0.84%, more preferably no more than about 0.83%, more preferably no more than about 0.82%, more preferably no more than about 0.81%, more preferably no more than about 0.80%, more preferably no more than about 0.79%, more preferably no more than about 0.78%, more preferably no more than about 0.77%, more preferably no more than about 0.76%, more preferably no more than about 0.75%, more preferably no more than about 0.74%, more preferably no more than about 0.73%, more preferably no more than about 0.72%, more preferably no more than about 0.71%, more preferably no more than about 0.97%, more preferably no more than about 0.69%, more preferably no more than about 0.68%, more preferably no more than about 0.67%, more preferably no more than about 0.66%, more preferably no more than about 0.65%, more preferably no more than about 0.64%, more preferably no more than about 0.63%, more preferably no more than about 0.62%, more preferably no more than about 0.61%, more preferably no more than about 0.60%, more preferably no more than about 0.59%, more preferably no more than about 0.58%, more preferably no more than about 0.57%, more preferably no more than about 0.56%, more preferably no more than about 0.55%, more preferably no more than about 0.54%, more preferably no more than about 0.53%, more preferably no more than about 0.52%, more preferably no more than about 0.51%, more preferably no more than about 0.50%, more preferably no more than about 0.49%, more preferably no more than about 0.48%, more preferably no more than about 0.47%, more preferably no more than about 0.46%, more preferably no more than about 0.45%, more preferably no more than about 0.44%, more preferably no more than about 0.43%, more preferably no more than about 0.42%, more preferably no more than about 0.41%, more preferably no more than about 0.40%, more preferably no more than about 0.39%, more preferably no more than about 0.38%, more preferably no more than about 0.37%, more preferably no more than about 0.36%, more preferably no more than about 0.35%, more preferably no more than about 0.34%, more preferably no more than about 0.33%, more preferably no more than about 0.32%, more preferably no more than about 0.31%, more preferably no more than about 0.30%, more preferably no more than about 0.29%, more preferably no more than about 0.28%, more preferably no more than about 0.27%, more preferably no more than about 0.26%, more preferably no more than about 0.25%, more preferably no more than about 0.24%, more preferably no more than about 0.23%, more preferably no more than about 0.22%, more preferably no more than about 0.21%, more preferably no more than about 0.20%, more preferably no more than about 0.19%, more preferably no more than about 0.18%, more preferably no more than about 0.17%, more preferably no more than about 0.16%, more preferably no more than about 0.15%, more preferably no more than about 0.14%, more preferably no more than about 0.13%, more preferably no more than about 0.12%, more preferably no more than about 0.11%, and most preferably no more than about 0.10%.

According to some aspects, the pharmaceutical composition of the present invention may have a total impurity concentration after a certain period of shelf life of no more than about 5%, more preferably no more than about 4.5%, more preferably no more than about 4%, more preferably no more than about 3.5%, more preferably no more than about 3%, more preferably no more than about 2.5%, more preferably no more than about 2%, more preferably no more than about 1.5%, more preferably no more than about 1%, and most preferably no more than about 0.5%.

According to some aspects, the pharmaceutical composition of the present invention may have a total impurity concentration after a certain period of shelf life of no more than about 1%, more preferably no more than about 0.99%, more preferably no more than about 0.98%, more preferably no more than about 0.97%, more preferably no more than about 0.96%, more preferably no more than about 0.95%, more preferably no more than about 0.94%, more preferably no more than about 0.93%, more preferably no more than about 0.92%, more preferably no more than about 0.91%, more preferably no more than about 0.90%, more preferably no more than about 0.89%, more preferably no more than about 0.88%, more preferably no more than about 0.87%, more preferably no more than about 0.86%, more preferably no more than about 0.85%, more preferably no more than about 0.84%, more preferably no more than about 0.83%, more preferably no more than about 0.82%, more preferably no more than about 0.81%, more preferably no more than about 0.80%, more preferably no more than about 0.79%, more preferably no more than about 0.78%, more preferably no more than about 0.77%, more preferably no more than about 0.76%, more preferably no more than about 0.75%, more preferably no more than about 0.74%, more preferably no more than about 0.73%, more preferably no more than about 0.72%, more preferably no more than about 0.71%, more preferably no more than about 0.97%, more preferably no more than about 0.69%, more preferably no more than about 0.68%, more preferably no more than about 0.67%, more preferably no more than about 0.66%, more preferably no more than about 0.65%, more preferably no more than about 0.64%, more preferably no more than about 0.63%, more preferably no more than about 0.62%, more preferably no more than about 0.61%, more preferably no more than about 0.60%, more preferably no more than about 0.59%, more preferably no more than about 0.58%, more preferably no more than about 0.57%, more preferably no more than about 0.56%, more preferably no more than about 0.55%, more preferably no more than about 0.54%, more preferably no more than about 0.53%, more preferably no more than about 0.52%, more preferably no more than about 0.51%, more preferably no more than about 0.50%, more preferably no more than about 0.49%, more preferably no more than about 0.48%, more preferably no more than about 0.47%, more preferably no more than about 0.46%, more preferably no more than about 0.45%, more preferably no more than about 0.44%, more preferably no more than about 0.43%, more preferably no more than about 0.42%, more preferably no more than about 0.41%, more preferably no more than about 0.40%, more preferably no more than about 0.39%, more preferably no more than about 0.38%, more preferably no more than about 0.37%, more preferably no more than about 0.36%, more preferably no more than about 0.35%, more preferably no more than about 0.34%, more preferably no more than about 0.33%, more preferably no more than about 0.32%, more preferably no more than about 0.31%, more preferably no more than about 0.30%, more preferably no more than about 0.29%, more preferably no more than about 0.28%, more preferably no more than about 0.27%, more preferably no more than about 0.26%, more preferably no more than about 0.25%, more preferably no more than about 0.24%, more preferably no more than about 0.23%, more preferably no more than about 0.22%, more preferably no more than about 0.21%, more preferably no more than about 0.20%, more preferably no more than about 0.19%, more preferably no more than about 0.18%, more preferably no more than about 0.17%, more preferably no more than about 0.16%, more preferably no more than about 0.15%, more preferably no more than about 0.14%, more preferably no more than about 0.13%, more preferably no more than about 0.12%, more preferably no more than about 0.11%, and most preferably no more than about 0.10%. The formulations of the present invention, when stored for at least three months at room temperature, the formulation has: at least about 99% pure pitolisant and less than about 1% of total degradant impurities.

According to some aspects, the pharmaceutical composition of the present invention may have an initial Pitolisant N-oxide impurity concentration of no more than about 5%, more preferably no more than about 4.5%, more preferably no more than about 4%, more preferably no more than about 3.5%, more preferably no more than about 3%, more preferably no more than about 2.5%, more preferably no more than about 2%, more preferably no more than about 1.5%, more preferably no more than about 1%. In a most preferred aspect, the formulation contains less than 0.5% pitolisant N-oxide impurity.

According to some aspects, the pharmaceutical composition of the present invention may have an initial Pitolisant N-oxide impurity concentration of no more than about 1%, more preferably no more than about 0.99%, more preferably no more than about 0.98%, more preferably no more than about 0.97%, more preferably no more than about 0.96%, more preferably no more than about 0.95%, more preferably no more than about 0.94%, more preferably no more than about 0.93%, more preferably no more than about 0.92%, more preferably no more than about 0.91%, more preferably no more than about 0.90%, more preferably no more than about 0.89%, more preferably no more than about 0.88%, more preferably no more than about 0.87%, more preferably no more than about 0.86%, more preferably no more than about 0.85%, more preferably no more than about 0.84%, more preferably no more than about 0.83%, more preferably no more than about 0.82%, more preferably no more than about 0.81%, more preferably no more than about 0.80%, more preferably no more than about 0.79%, more preferably no more than about 0.78%, more preferably no more than about 0.77%, more preferably no more than about 0.76%, more preferably no more than about 0.75%, more preferably no more than about 0.74%, more preferably no more than about 0.73%, more preferably no more than about 0.72%, more preferably no more than about 0.71%, more preferably no more than about 0.97%, more preferably no more than about 0.69%, more preferably no more than about 0.68%, more preferably no more than about 0.67%, more preferably no more than about 0.66%, more preferably no more than about 0.65%, more preferably no more than about 0.64%, more preferably no more than about 0.63%, more preferably no more than about 0.62%, more preferably no more than about 0.61%, more preferably no more than about 0.60%, more preferably no more than about 0.59%, more preferably no more than about 0.58%, more preferably no more than about 0.57%, more preferably no more than about 0.56%, more preferably no more than about 0.55%, more preferably no more than about 0.54%, more preferably no more than about 0.53%, more preferably no more than about 0.52%, more preferably no more than about 0.51%, more preferably no more than about 0.50%, more preferably no more than about 0.49%, more preferably no more than about 0.48%, more preferably no more than about 0.47%, more preferably no more than about 0.46%, more preferably no more than about 0.45%, more preferably no more than about 0.44%, more preferably no more than about 0.43%, more preferably no more than about 0.42%, more preferably no more than about 0.41%, more preferably no more than about 0.40%, more preferably no more than about 0.39%, more preferably no more than about 0.38%, more preferably no more than about 0.37%, more preferably no more than about 0.36%, more preferably no more than about 0.35%, more preferably no more than about 0.34%, more preferably no more than about 0.33%, more preferably no more than about 0.32%, more preferably no more than about 0.31%, more preferably no more than about 0.30%, more preferably no more than about 0.29%, more preferably no more than about 0.28%, more preferably no more than about 0.27%, more preferably no more than about 0.26%, more preferably no more than about 0.25%, more preferably no more than about 0.24%, more preferably no more than about 0.23%, more preferably no more than about 0.22%, more preferably no more than about 0.21%, more preferably no more than about 0.20%, more preferably no more than about 0.19%, more preferably no more than about 0.18%, more preferably no more than about 0.17%, more preferably no more than about 0.16%, more preferably no more than about 0.15%, more preferably no more than about 0.14%, more preferably no more than about 0.13%, more preferably no more than about 0.12%, more preferably no more than about 0.11%, more preferably no more than about 0.09%, more preferably no more than about 0.08%, more preferably no more than about 0.07%, more preferably no more than about 0.06%, more preferably no more than about 0.05%, more preferably no more than about 0.04%, more preferably no more than about 0.03%, more preferably no more than about 0.02%, and most preferably no more than about 0.01%.

According to some aspects, the pharmaceutical composition of the present invention may have Pitolisant N-oxide impurity concentration after a certain period of shelf life of no more than about 5%, more preferably no more than about 4.5%, more preferably no more than about 4%, more preferably no more than about 3.5%, more preferably no more than about 3%, more preferably no more than about 2.5%, more preferably no more than about 2%, more preferably no more than about 1.5%, more preferably no more than about 1%, and most preferably no more than about 0.5%.

According to some aspects, the pharmaceutical composition of the present invention may have Pitolisant N-oxide impurity concentration after a certain period of shelf life of no more than about 1%, more preferably no more than about 0.99%, more preferably no more than about 0.98%, more preferably no more than about 0.97%, more preferably no more than about 0.96%, more preferably no more than about 0.95%, more preferably no more than about 0.94%, more preferably no more than about 0.93%, more preferably no more than about 0.92%, more preferably no more than about 0.91%, more preferably no more than about 0.90%, more preferably no more than about 0.89%, more preferably no more than about 0.88%, more preferably no more than about 0.87%, more preferably no more than about 0.86%, more preferably no more than about 0.85%, more preferably no more than about 0.84%, more preferably no more than about 0.83%, more preferably no more than about 0.82%, more preferably no more than about 0.81%, more preferably no more than about 0.80%, more preferably no more than about 0.79%, more preferably no more than about 0.78%, more preferably no more than about 0.77%, more preferably no more than about 0.76%, more preferably no more than about 0.75%, more preferably no more than about 0.74%, more preferably no more than about 0.73%, more preferably no more than about 0.72%, more preferably no more than about 0.71%, more preferably no more than about 0.97%, more preferably no more than about 0.69%, more preferably no more than about 0.68%, more preferably no more than about 0.67%, more preferably no more than about 0.66%, more preferably no more than about 0.65%, more preferably no more than about 0.64%, more preferably no more than about 0.63%, more preferably no more than about 0.62%, more preferably no more than about 0.61%, more preferably no more than about 0.60%, more preferably no more than about 0.59%, more preferably no more than about 0.58%, more preferably no more than about 0.57%, more preferably no more than about 0.56%, more preferably no more than about 0.55%, more preferably no more than about 0.54%, more preferably no more than about 0.53%, more preferably no more than about 0.52%, more preferably no more than about 0.51%, more preferably no more than about 0.50%, more preferably no more than about 0.49%, more preferably no more than about 0.48%, more preferably no more than about 0.47%, more preferably no more than about 0.46%, more preferably no more than about 0.45%, more preferably no more than about 0.44%, more preferably no more than about 0.43%, more preferably no more than about 0.42%, more preferably no more than about 0.41%, more preferably no more than about 0.40%, more preferably no more than about 0.39%, more preferably no more than about 0.38%, more preferably no more than about 0.37%, more preferably no more than about 0.36%, more preferably no more than about 0.35%, more preferably no more than about 0.34%, more preferably no more than about 0.33%, more preferably no more than about 0.32%, more preferably no more than about 0.31%, more preferably no more than about 0.30%, more preferably no more than about 0.29%, more preferably no more than about 0.28%, more preferably no more than about 0.27%, more preferably no more than about 0.26%, more preferably no more than about 0.25%, more preferably no more than about 0.24%, more preferably no more than about 0.23%, more preferably no more than about 0.22%, more preferably no more than about 0.21%, more preferably no more than about 0.20%, more preferably no more than about 0.19%, more preferably no more than about 0.18%, more preferably no more than about 0.17%, more preferably no more than about 0.16%, more preferably no more than about 0.15%, more preferably no more than about 0.14%, more preferably no more than about 0.13%, more preferably no more than about 0.12%, more preferably no more than about 0.11%, more preferably no more than about 0.09%, more preferably no more than about 0.08%, more preferably no more than about 0.07%, more preferably no more than about 0.06%, more preferably no more than about 0.05%, more preferably no more than about 0.04%, more preferably no more than about 0.03%, more preferably no more than about 0.02%, and most preferably no more than about 0.01%.

### Pharmacokinetic Embodiments

In some embodiments, after oral administration of the pitolisant liquid formulation of the present invention at doses between about 1 mg and about 200 mg to human subjects, the AUC_{0-∞} (area under the plasma drug concentration-time curve extrapolated to infinity) for pitolisant may be observed in the range of about 5 ng·h/L to about 10,000 ng·h/L.

In some embodiments, after oral administration of the pitolisant liquid formulation of the present invention at dose of about 1 mg to human subjects, the AUC_{0-∞} for pitolisant may be observed in the range of about 5 ng·h/L to about 50 ng·h/L.

In some embodiments, after oral administration of the pitolisant liquid formulation of the present invention at dose of about 5 mg to human subjects, the AUC_{0-∞} for pitolisant may be observed in the range of about 25 ng·h/L to about 250 ng·h/L.

In some embodiments, after oral administration of the pitolisant liquid formulation of the present invention at dose of about 10 mg to human subjects, the AUC_{0-∞} for pitolisant may be observed in the range of about 50 ng·h/L to about 500 ng·h/L.

In some embodiments, after oral administration of the pitolisant liquid formulation of the present invention at dose of about 20 mg to human subjects, the AUC_{0-∞} for pitolisant may be observed in the range of about 100 ng·h/L to about 1,000 ng·h/L.

In some embodiments, after oral administration of the pitolisant liquid formulation of the present invention at dose of about 40 mg to human subjects, the AUC_{0-∞} for pitolisant may be observed in the range of about 200 ng·h/L to about 2,000 ng·h/L.

In some embodiments, after oral administration of the pitolisant liquid formulation of the present invention at dose of about 60 mg to human subjects, the AUC_{0-∞} for pitolisant may be observed in the range of about 300 ng·h/L to about 3,000 ng·h/L.

In some embodiments, after oral administration of the pitolisant liquid formulation of the present invention at dose of about 80 mg to human subjects, the AUC_{0-∞} for pitolisant may be observed in the range of about 400 ng·h/L to about 4,000 ng·h/L.

In some embodiments, after oral administration of the pitolisant liquid formulation of the present invention at dose of about 120 mg to human subjects, the AUC_{0-∞} for pitolisant may be observed in the range of about 600 ng·h/L to about 6,000 ng·h/L.

In some embodiments, after oral administration of the pitolisant liquid formulation of the present invention at dose of about 160 mg to human subjects, the AUC_{0-∞} for pitolisant may be observed in the range of about 800 ng·h/L to about 8,000 ng·h/L.

In some embodiments, after oral administration of the pitolisant liquid formulation of the present invention at dose of about 180 mg to human subjects, the AUC_{0-∞} for pitolisant may be observed in the range of about 900 ng·h/L to about 9,000 ng·h/L.

In some embodiments, after oral administration of the pitolisant liquid formulation of the present invention at dose of about 200 mg to human subjects, the AUC_{0-∞} for pitolisant may be observed in the range of about 1,000 ng·h/L to about 10,000 ng·h/L.

In some embodiments, after oral administration of the pitolisant liquid formulation of the present invention at doses between about 1 mg and about 200 mg to human subjects, the Cₘₐₓ (maximum plasma drug concentration) for pitolisant may be observed in the range of about 1 ng/ml to about 1,000 ng/mL.

In some embodiments, after oral administration of the pitolisant liquid formulation of the present invention at dose of about 1 mg to human subjects, the Cₘₐₓ (maximum plasma drug concentration) for pitolisant may be observed in the range of about 1 ng/ml to about 5 ng/mL.

In some embodiments, after oral administration of the pitolisant liquid formulation of the present invention at dose of about 5 mg to human subjects, the Cₘₐₓ (maximum plasma drug concentration) for pitolisant may be observed in the range of about 5 ng/ml to about 25 ng/ml.

In some embodiments, after oral administration of the pitolisant liquid formulation of the present invention at dose of about 10 mg to human subjects, the Cₘₐₓ (maximum plasma drug concentration) for pitolisant may be observed in the range of about 10 ng/mL to about 50 ng/mL.

In some embodiments, after oral administration of the pitolisant liquid formulation of the present invention at dose of about 20 mg to human subjects, the Cₘₐₓ (maximum plasma drug concentration) for pitolisant may be observed in the range of about 20 ng/mL to about 100 ng/mL.

In some embodiments, after oral administration of the pitolisant liquid formulation of the present invention at dose of about 40 mg to human subjects, the Cₘₐₓ (maximum plasma drug concentration) for pitolisant may be observed in the range of about 40 ng/ml to about 200 ng/mL.

In some embodiments, after oral administration of the pitolisant liquid formulation of the present invention at dose of about 60 mg to human subjects, the Cₘₐₓ (maximum plasma drug concentration) for pitolisant may be observed in the range of about 60 ng/ml to about 300 ng/mL.

In some embodiments, after oral administration of the pitolisant liquid formulation of the present invention at dose of about 80 mg to human subjects, the Cₘₐₓ (maximum plasma drug concentration) for pitolisant may be observed in the range of about 80 ng/ml to about 400 ng/mL.

In some embodiments, after oral administration of the pitolisant liquid formulation of the present invention at dose of about 120 mg to human subjects, the Cₘₐₓ (maximum plasma drug concentration) for pitolisant may be observed in the range of about 120 ng/ml to about 600 ng/mL.

In some embodiments, after oral administration of the pitolisant liquid formulation of the present invention at dose of about 160 mg to human subjects, the Cₘₐₓ (maximum plasma drug concentration) for pitolisant may be observed in the range of about 160 ng/mL to about 800 ng/mL.

In some embodiments, after oral administration of the pitolisant liquid formulation of the present invention at dose of about 180 mg to human subjects, the Cₘₐₓ (maximum plasma drug concentration) for pitolisant may be observed in the range of about 180 ng/ml to about 900 ng/mL.

In some embodiments, after oral administration of the pitolisant liquid formulation of the present invention at dose of about 200 mg to human subjects, the Cₘₐₓ (maximum plasma drug concentration) for pitolisant may be observed in the range of about 200 ng/mL to about 1,000 ng/mL.

In some embodiments, after oral administration of the pitolisant liquid formulation of the present invention, the Tₘₐₓ (time to reach maximum plasma drug concentration in human) for pitolisant may be observed in the range from about 1 hour to about 8 hours, from about 1 hour to about 6 hours, from about 2 hour to about 6 hours, from about 2 hours to about 5 hours, about 1 hour, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, or about 8 hours.

In some embodiments, after intranasal administration of the pitolisant liquid formulation of the present invention at doses between about 0.5 mg and about 100 mg to human subjects, the AUC_{0-∞} (area under the plasma drug concentration-time curve extrapolated to infinity) for pitolisant may be observed in the range of about 5 ng·h/L to about 10,000 ng·h/L.

In some embodiments, after intranasal administration of the pitolisant liquid formulation of the present invention at a dose of about 0.5 mg to human subjects, the AUC_{0-∞} for pitolisant may be observed in the range of about 5 ng·h/L to about 50 ng·h/L.

In some embodiments, after intranasal administration of the pitolisant liquid formulation of the present invention at a dose of about 2.5 mg to human subjects, the AUC_{0-∞} for pitolisant may be observed in the range of about 25 ng·h/L to about 250 ng·h/L.

In some embodiments, after intranasal administration of the pitolisant liquid formulation of the present invention at a dose of about 5 mg to human subjects, the AUC_{0-∞} for pitolisant may be observed in the range of about 50 ng·h/L to about 500 ng·h/L.

In some embodiments, after intranasal administration of the pitolisant liquid formulation of the present invention at a dose of about 10 mg to human subjects, the AUC_{0-∞} for pitolisant may be observed in the range of about 100 ng·h/L to about 1,000 ng·h/L.

In some embodiments, after intranasal administration of the pitolisant liquid formulation of the present invention at a dose of about 20 mg to human subjects, the AUC_{0-∞} for pitolisant may be observed in the range of about 200 ng·h/L to about 2,000 ng·h/L.

In some embodiments, after intranasal administration of the pitolisant liquid formulation of the present invention at a dose of about 30 mg to human subjects, the AUC_{0-∞} for pitolisant may be observed in the range of about 300 ng·h/L to about 3,000 ng·h/L.

In some embodiments, after intranasal administration of the pitolisant liquid formulation of the present invention at a dose of about 40 mg to human subjects, the AUC_{0-∞} for pitolisant may be observed in the range of about 400 ng·h/L to about 4,000 ng·h/L.

In some embodiments, after intranasal administration of the pitolisant liquid formulation of the present invention at a dose of about 60 mg to human subjects, the AUC_{0-∞} for pitolisant may be observed in the range of about 600 ng·h/L to about 6,000 ng·h/L.

In some embodiments, after intranasal administration of the pitolisant liquid formulation of the present invention at a dose of about 80 mg to human subjects, the AUC_{0-∞} for pitolisant may be observed in the range of about 800 ng·h/L to about 8,000 ng·h/L.

In some embodiments, after intranasal administration of the pitolisant liquid formulation of the present invention at a dose of about 100 mg to human subjects, the AUC_{0-∞} for pitolisant may be observed in the range of about 1,000 ng·h/L to about 10,000 ng·h/L.

In some embodiments, after intranasal administration of the pitolisant liquid formulation of the present invention at doses between about 0.5 mg and about 100 mg to human subjects, the Cₘₐₓ (maximum plasma drug concentration) for pitolisant may be observed in the range of about 1 ng/ml to about 1,000 ng/mL.

In some embodiments, after intranasal administration of the pitolisant liquid formulation of the present invention at a dose of about 0.5 mg to human subjects, the Cₘₐₓ for pitolisant may be observed in the range of about 1 ng/ml to about 5 ng/mL.

In some embodiments, after intranasal administration of the pitolisant liquid formulation of the present invention at a dose of about 2.5 mg to human subjects, the Cₘₐₓ for pitolisant may be observed in the range of about 5 ng/ml to about 25 ng/mL.

In some embodiments, after intranasal administration of the pitolisant liquid formulation of the present invention at a dose of about 5 mg to human subjects, the Cₘₐₓ for pitolisant may be observed in the range of about 10 ng/mL to about 50 ng/mL.

In some embodiments, after intranasal administration of the pitolisant liquid formulation of the present invention at a dose of about 10 mg to human subjects, the Cₘₐₓ for pitolisant may be observed in the range of about 20 ng/ml to about 100 ng/mL.

In some embodiments, after intranasal administration of the pitolisant liquid formulation of the present invention at a dose of about 20 mg to human subjects, the Cₘₐₓ for pitolisant may be observed in the range of about 40 ng/ml to about 200 ng/mL.

In some embodiments, after intranasal administration of the pitolisant liquid formulation of the present invention at a dose of about 30 mg to human subjects, the Cₘₐₓ for pitolisant may be observed in the range of about 60 ng/mL to about 300 ng/ml.

In some embodiments, after intranasal administration of the pitolisant liquid formulation of the present invention at a dose of about 40 mg to human subjects, the Cₘₐₓ for pitolisant may be observed in the range of about 80 ng/ml to about 400 ng/mL.

In some embodiments, after intranasal administration of the pitolisant liquid formulation of the present invention at a dose of about 60 mg to human subjects, the Cₘₐₓ for pitolisant may be observed in the range of about 120 ng/ml to about 600 ng/mL.

In some embodiments, after intranasal administration of the pitolisant liquid formulation of the present invention at a dose of about 80 mg to human subjects, the Cₘₐₓ for pitolisant may be observed in the range of about 160 ng/ml to about 800 ng/mL.

In some embodiments, after intranasal administration of the pitolisant liquid formulation of the present invention at a dose of about 90 mg to human subjects, the Cₘₐₓ for pitolisant may be observed in the range of about 180 ng/ml to about 900 ng/ml.

In some embodiments, after intranasal administration of the pitolisant liquid formulation of the present invention at a dose of about 100 mg to human subjects, the Cₘₐₓ for pitolisant may be observed in the range of about 200 ng/mL to about 1,000 ng/mL.

In some embodiments, after intranasal administration of the pitolisant liquid formulation of the present invention, the Tₘₐₓ (time to reach maximum plasma drug concentration in human) for pitolisant may be observed in the range from about 5 minutes to about 6 hours, from about 5 minutes to about 4 hours, from about 10 minutes to about 3 hours, from about 10 minutes to about 1 hours, from about 15 minutes to about 2 hours, from about 30 minutes to about 1 hour, about 5 minutes, about 10 minutes, about 15 minutes, about 20 minutes, about 25 minutes, about 30 minutes, about 45 minutes, about 1 hour, about 1.25 hours, about 1.5 hours, about 1.75 hours, about 2 hours, about 2.5 hours, about 3 hours, about 3.5 hours, about 4 hours, about 4.5 hours, about 5 hours, about 5.5 hours, or about 6 hours.

In some embodiments, the invention provides a method for treating excessive daytime sleepiness (EDS) in a subject with narcolepsy, the method comprising administering to the subject a therapeutically effective amount of an oral liquid formulation of pitolisant, or a pharmaceutically acceptable salt thereof. The administration of the oral liquid formulation results in a therapeutic outcome that is equivalent to the outcome achieved by administering an oral solid tablet formulation containing a similar daily dose of pitolisant.

In some embodiments, the therapeutically effective amount of the oral liquid formulation comprises a daily dose of pitolisant base selected from the group consisting of about 4.45 mg, about 8.9 mg, about 17.8 mg, about 35.6 mg, about 71.2 mg, about 106.8 mg, about 142.4 mg, about 160.2 mg, and about 178 mg. These doses are equivalent to a daily dose of pitolisant hydrochloride of about 5 mg, about 10 mg, about 20 mg, about 40 mg, about 80 mg, about 120 mg, about 160 mg, about 180 mg, and about 200 mg, respectively. The dose may be titrated to achieve an optimal clinical response.

In some embodiments, the equivalent therapeutic outcome is defined as a reduction from baseline in the Epworth Sleepiness Scale (ESS) score. The reduction achieved by the oral liquid formulation is statistically non-inferior to the reduction achieved by an oral solid tablet formulation administered at an equivalent dose.

In some embodiments of the present invention, the administration of the oral liquid formulation at a daily dose selected from the group consisting of about 4.45 mg, about 8.9 mg, about 17.8 mg, about 35.6 mg, about 71.2 mg, about 106.8 mg, about 142.4 mg, about 160.2 mg, and about 178 mg of pitolisant base (equivalent to about 5 mg, about 10 mg, about 20 mg, about 40 mg, about 80 mg, about 120 mg, about 160 mg, about 180 mg, and about 200 mg of pitolisant hydrochloride, respectively) results in a mean decrease from baseline of at least 3 points on the Epworth Sleepiness Scale (ESS) in a population of subjects, particularly when said subjects are titrated to a stable and effective maintenance dose.

In some aspects, the present invention provides a oral liquid dosage form with a predictable pharmacokinetic profile upon single-dose administration to human subjects. In certain aspects, the invention provides an oral liquid composition that is bioequivalent to an oral solid dosage form. The following embodiments further detail the pharmacokinetic characteristics of the present invention across a range of dosage strengths.

It is contemplated that a clinical study will be conducted to demonstrate the bioequivalence of a oral liquid pharmaceutical composition of the present invention relative to a reference listed drug product. The study is designed as a randomized, open-label, single-dose, two-period, two-treatment crossover study in a cohort of about 24 to 48 healthy adult subjects under fasting conditions. In the first period, subjects are randomly assigned to receive either a single oral dose of the test product (a liquid composition of present invention containing about 20 mg to about 180 mg of pitolisant hydrochloride) or the reference product of the same strength. Following a washout period of about 14 days, subjects receive alternate treatment in the second period. Serial blood samples for pharmacokinetic analysis are collected at pre-dose (0 hours) and at 0.5, 1, 1.5, 2, 2.5, 3, 3.5, 4, 5, 6, 8, 10, 12, 14, 16, 20, 24, 36, 48, and 72-hours post-dose. Plasma concentrations of pitolisant are determined using a validated LC-MS/MS method or other suitable methods. The pharmacokinetic parameters, including AUC₀₋ₜ, AUC_{0-∞}, and Cₘₐₓ, are calculated for both formulations. It is expected that the 90% confidence intervals for the geometric mean ratios of the test product to the reference product will fall entirely within the 80% to 125% acceptance range for both AUC_{0-∞} and Cₘₐₓ, thereby demonstrating the bioequivalence of the liquid composition.

In addition to a single dose, fasting, crossover study, other scientifically valid designs may be used to establish the bioequivalence of a oral liquid pitolisant compositions. These alternatives allow for the assessment of the drug's performance under various conditions. One alternative is a multiple-dose, steady-state crossover study. This design assesses bioequivalence under conditions that reflect drug accumulation following the repeated dosing characteristic of pitolisant's therapeutic regimen. Another alternative is a study conducted under fed conditions. This design evaluates the effect of food on the drug's bioavailability and can be used to establish bioequivalence when the composition is administered with a meal. Regardless of the specific design employed, the objective is to demonstrate bioequivalence by showing that the 90% confidence interval for the ratio of the geometric means of the key pharmacokinetic parameters (AUC and Cₘₐₓ) falls within the standard acceptance range of about 80% to about 125%.

In one embodiment, the present invention relates to a liquid pharmaceutical composition comprising about 20 mg to about 180 mg of pitolisant in a volume ranging from about 1 mL to about 40 mL. Following single-dose administration to healthy human subjects, this liquid composition could demonstrate bioequivalence to an oral reference tablet, as evidenced by a single-dose geometric mean Area Under the Curve from time zero to infinity (AUC_{0-∞}) and a geometric mean Maximum Plasma Concentration (Cₘₐₓ). Bioequivalence is established when the 90% confidence interval for the ratio of geometric means for both AUC_{0-∞} and Cₘₐₓ falls entirely within the range of about 80% to about 125%.

In some embodiments of the present invention, the invention provides a method for treating cataplexy in a subject with narcolepsy. The method comprises administering an oral liquid formulation of pitolisant, wherein the administration results in a reduction in the mean weekly rate of cataplexy (WRC) that is statistically equivalent to the reduction achieved by administering an oral solid tablet formulation at a similar daily dose.

In some embodiments of the present invention, the administration of the oral liquid formulation at a daily dose selected from the group consisting of about 4.45 mg, about 8.9 mg, about 17.8 mg, about 35.6 mg, about 71.2 mg, about 106.8 mg, about 142.4 mg, and about 178 mg of pitolisant base (equivalent to about 5 mg, about 10 mg, about 20 mg, about 40 mg, about 80 mg, about 120 mg, about 160 mg, about 180 mg, and about 200 mg of pitolisant hydrochloride, respectively) results in a mean reduction from baseline in the weekly rate of cataplexy of at least 50% in a population of subjects, particularly when said subjects are titrated to a stable and effective maintenance dose. In some embodiments, the present invention relates to methods for treating a condition responsive to H3 receptor antagonism, such as excessive daytime sleepiness (EDS) and cataplexy associated with narcolepsy, in a human subject in need thereof. The method comprises the intranasal administration of a pharmaceutical composition of pitolisant. The following non-limiting examples describe clinical trial designs intended to demonstrate the efficacy and safety of said intranasal formulation.

In one embodiment, a Phase 3 clinical study could be designed as a multicenter, randomized, double-blind, placebo-controlled, parallel-group trial to evaluate the efficacy and safety of an intranasal pitolisant formulation in adult subjects with narcolepsy. The study population comprises subjects aged 6 years of age and older with a diagnosis of narcolepsy (with or without cataplexy) confirmed by polysomnography and a Multiple Sleep Latency Test (MSLT), and who have a baseline Epworth Sleepiness Scale (ESS) score of 12 or greater. Following a screening period and a 2-week baseline data collection period, approximately 200 subjects are randomized in a 1:1 ratio to receive either the intranasal pitolisant formulation or a matching intranasal placebo. The treatment period has a duration of about 10 weeks, comprising a 4-week dose-optimization phase followed by a 6-week stable-dose phase. During the optimization phase, subjects are titrated from a starting dose of about 9 mg daily to a clinically optimal and well-tolerated dose, not to exceed about 36 mg daily. The primary efficacy endpoint is the change from baseline to the end of the stable-dose period (Week 10) in the ESS score. Key secondary endpoints include the change from baseline in mean sleep latency on the Maintenance of Wakefulness Test (MWT), the Patient Global Impression of Severity (PGI-S), and the weekly rate of cataplexy (WRC) in subjects with a history of cataplexy.

In another embodiment, the clinical study could be an active-controlled, randomized, double-blind, non-inferiority trial designed to compare the efficacy of the intranasal pitolisant formulation to the standard-of-care oral pitolisant tablet. The objective is to demonstrate that the intranasal formulation is at least as effective as the approved oral formulation. Subjects meeting the inclusion criteria for narcolepsy are randomized to receive either the intranasal pitolisant or the oral pitolisant tablet with a matching oral placebo. A double-dummy design is employed to maintain the blind, wherein each subject receives both an active intranasal spray and an oral placebo, or a placebo intranasal spray and an active oral tablet. The treatment duration is about 12 weeks, including a flexible titration schedule. The primary endpoint is the change from baseline in the ESS score at Week 12. Non-inferiority will be established if the upper bound of the 95% confidence interval for the difference in ESS score change from baseline between the intranasal and oral treatment groups is less than a pre-specified non-inferiority margin, for example, 2.5 points. Secondary endpoints may include assessments of patient preference and speed of onset of therapeutic effect.

In yet another embodiment, the invention provides for a three-arm, placebo- and active-controlled clinical study to simultaneously establish superiority over placebo and characterize the therapeutic profile relative to the oral tablet. Approximately 300 subjects are randomized in a 2:2:1 ratio to one of three arms: (1) intranasal pitolisant, (2) oral pitolisant tablet, or (3) placebo. The study duration is 12 weeks. The primary efficacy endpoint is the change from baseline in the mean sleep latency as measured by the MWT, providing an objective measure of the ability to remain awake. A hierarchical statistical testing procedure is employed, first testing the superiority of intranasal pitolisant to placebo. If this comparison is statistically significant, a subsequent test for the non-inferiority or superiority of the intranasal formulation to the oral tablet is performed.

In a further embodiment, the clinical trial could be designed to specifically evaluate the anti-cataplectic effects of the intranasal pitolisant formulation. The study population is limited to subjects with Narcolepsy Type 1 who experience a high frequency of cataplexy, defined as a minimum of four cataplexy events per week during the baseline period, as recorded in a patient diary. The study is a randomized, placebo-controlled withdrawal design. In this design, all subjects are initially treated with open-label intranasal pitolisant during a 12-week optimization and stabilization phase. Subjects who demonstrate a clinically meaningful response are then randomized in a double-blind fashion to either continue treatment with their stable dose of intranasal pitolisant or be switched to a matching placebo for a period of about 4 weeks. The primary efficacy endpoint is the time to relapse, defined as a significant worsening of cataplexy.

In another embodiment, the invention provides for a long-term, open-label extension (OLE) study to assess the safety and durability of the treatment effect of the intranasal pitolisant formulation. Subjects who have completed any of the preceding controlled Phase 3 studies are eligible to enroll. All subjects in the OLE study receive active intranasal pitolisant for a duration of about 12 to 24 months. The primary endpoints are the incidence, severity, and type of treatment-emergent adverse events, with a particular focus on long-term local nasal safety and tolerability as assessed by periodic nasopharyngeal examinations. Secondary endpoints include the sustained improvement in ESS, MWT, and WRC scores over the long-term treatment period to demonstrate the durability of the clinical benefit.

In some embodiments of the present invention, therapeutic equivalence could be established by achieving a bioequivalent pharmacokinetic (PK) profile. The oral liquid formulation, when administered at a dose strength selected from the group consisting of about 4.45 mg of pitolisant base (equivalent to about 5 mg of pitolisant hydrochloride), about 8.9 mg of pitolisant base (equivalent to about 10 mg of pitolisant hydrochloride), about 17.8 mg of pitolisant base (equivalent to about 20 mg of pitolisant hydrochloride), about 35.6 mg of pitolisant base (equivalent to about 40 mg of pitolisant hydrochloride), about 71.2 mg of pitolisant base (equivalent to about 80 mg of pitolisant hydrochloride), about 106.8 mg of pitolisant base (equivalent to about 120 mg of pitolisant hydrochloride), about 142.4 mg of pitolisant base (equivalent to about 160 mg of pitolisant hydrochloride), about 160.2 mg of pitolisant base (equivalent to about 180 mg of pitolisant hydrochloride), and about 178 mg of pitolisant base (equivalent to about 200 mg of pitolisant hydrochloride), provides a mean area under the plasma concentration-time curve (AUC_{0-∞}) and a mean maximum plasma concentration (Cₘₐₓ) where the 90% confidence intervals for the ratio of the geometric means between the liquid and tablet formulations fall within the range of 80% to 125%.

In certain embodiments, the invention provides a method for treating excessive daytime sleepiness (EDS) in a subject with narcolepsy. The method comprises administering an intranasal formulation of pitolisant, particularly as a primary morning dose, wherein said administration results in a superior clinical outcome compared to the administration of a standard oral solid tablet formulation. This superiority is demonstrated even when the total daily intranasal dose is substantially similar to or lower than the subject's total daily oral dose.

In a specific embodiment, the superior clinical outcome is defined as a statistically significant reduction in the time to achieve therapeutic wakefulness after the morning dose. For example, the administration of a single 17.8 mg intranasal dose of pitolisant base upon waking results in a clinically meaningful improvement in alertness, as measured by the Maintenance of Wakefulness Test (MWT), within 15 minutes. In contrast, an equivalent 17.8 mg oral dose requires over 60 minutes to produce a similar effect, leaving the subject impaired by severe sleep inertia for a prolonged period. This superior and rapid therapeutic effect is attributed to the pharmacokinetic profile achievable only through intranasal delivery. The formulation is designed for rapid absorption across the nasal mucosa, enabling the achievement of therapeutic plasma concentrations at site of action within minutes of administration. This provides a critical advantage for overcoming severe morning sleep inertia and accelerating the transition to full daytime wakefulness, an outcome not achievable with oral formulations, which are limited by the rate of gastrointestinal absorption. The intranasal administration thereby provides a novel therapeutic option that significantly improves morning function and may allow for a lower total daily dose to maintain alertness throughout the day.

In certain embodiments, the invention provides a method for treating excessive daytime sleepiness (EDS) in a subject with narcolepsy. The method comprises administering a daily dose of a liquid formulation of pitolisant via an intranasal route, wherein said administration results in a superior clinical outcome characterized by enhanced efficacy and an improved safety profile. This superiority is achieved while simultaneously reducing total systemic drug exposure when compared to a therapeutically effective oral solid tablet formulation.

In a specific embodiment, the superior clinical outcome is defined by a greater mean reduction from baseline in the Epworth Sleepiness Scale (ESS) score, coupled with lower systemic plasma concentrations. For example, after 8 weeks of therapy, subjects administered the intranasal formulation at a mean dose of 17.8 mg of pitolisant base achieve a mean reduction of 7.5 points on the ESS. Pharmacokinetic analysis confirms that this robust clinical effect is achieved with a mean area under the plasma concentration-time curve (AUC) approximately 40% lower than that of subjects administered a higher, 35.6 mg oral dose, who achieve a less effective mean ESS reduction of 4.8 points. This superior therapeutic profile is attributed to the formulation's capacity for direct nose-to-brain transport via the olfactory and trigeminal neural pathways. This mechanism allows the formulation to substantially bypass the blood-brain barrier and achieve a higher concentration of pitolisant directly at the histamine H3 receptor sites within the central nervous system. Simultaneously, it minimizes systemic circulation and overall drug burden, thereby reducing the potential for off-target side effects (prolonged QT interval) and suitable for patients with severe hepatic impairment and/or end-stage renal disease (ESRD). The intranasal administration thereby provides a more targeted and efficient treatment for EDS, enhancing efficacy while improving the overall safety and tolerability profile compared to standard oral therapy. Similar plausible embodiments may be applicable to other diseases treatable by pitolisant such as narcolepsy, excessive daytime sleepiness (EDS), EDS associated with obstructive sleep apnea, idiopathic hypersomnia, Prader-Willi Syndrome, Parkinson's disease, Attention-Deficit/Hyperactivity Disorder (ADHD), schizophrenia, Alzheimer's disease, depression, cognitive impairment, myotonic dystrophy, Dravet syndrome, Lennox-Gastaut syndrome, Rett syndrome, Tourette syndrome, and binge eating disorder.

### Method of Use Embodiments

In some embodiments of the present invention, an oral liquid pharmaceutical composition is provided, comprising: (a) pitolisant, or a pharmaceutically acceptable salt thereof, at a concentration sufficient to deliver a unit dose selected from a group consisting of about 4.45 mg, about 8.9 mg, about 17.8 mg, about 35.6 mg, about 71.2 mg, about 106.8 mg, about 142.4 mg, and about 178 mg of pitolisant base (equivalent to about 5 mg, about 10 mg, about 20 mg, about 40 mg, about 80 mg, about 120 mg, about 160 mg, about 180 mg, and about 200 mg of pitolisant hydrochloride, respectively); (b) at least one pharmaceutically acceptable liquid carrier; and (c) one or more excipients, wherein the dose volume is from about 0.1 mL to about 30 mL. This composition is formulated to provide a pharmacokinetic and therapeutic profile equivalent to that of a solid oral dosage form of pitolisant at a similar dose.

For the purposes of the present invention, the term “manageable volume” as used herein is defined as a quantity of the oral liquid pharmaceutical composition that is suitable for convenient, safe, and accurate administration to a subject, particularly for self-administration in a single dosing event. The volume is selected to promote patient compliance and adherence to a prescribed therapeutic regimen by minimizing the physical and psychological burden of administration.

In an embodiment, the pitolisant liquid formulation of the present invention is administered at therapeutically effective dose to the patients in need thereof.

In some embodiments, the pitolisant liquid formulation of the present invention is administered to the patient in amount from about 1 mg to about 200 mg per dose, such as about 4.45 mg, about 5 mg, about 8.9 mg, about 10 mg, about 15 mg, about 17.8 mg, about 20 mg, about 25 mg, about 30 mg, about 35.6 mg, about 40 mg, about 50 mg, about 60 mg, about 80 mg, about 100 mg, about 120 mg, about 140 mg, about 160 mg, about 180 mg, or about 200 mg per dose.

In an embodiment, the present invention provides a method of treating excessive daytime sleepiness (EDS) and/or cataplexy in adult patients with narcolepsy comprising administering a therapeutically effective amount of the pitolisant liquid formulation.

In some embodiments, the invention provides a method of treating excessive daytime sleepiness (EDS) in pediatric patients of six years and older with narcolepsy comprising administering a therapeutically effective amount of the formulation of the present invention.

In some embodiments, the invention provides a method of treating Prader-Willi Syndrome comprising administering the formulation of the present invention to a patient in need thereof.

In one embodiment, the formulation is administered orally, in a volume of about 0.5 mL to about 40 mL delivering about 5 mg to about 200 mg pitolisant per dose. In some embodiments, such dose may be delivered as liquid filled capsule for immediate release or delayed release, preferably delayed release.

In another embodiment, the formulation is administered as a spray via a route selected from oral, sublingual, or buccal, each spray comprising about 0.05 mL to about 0.2 mL and delivering about 5 mg to about 200 mg pitolisant per dose. In yet another embodiment, the formulation is administered intranasally, each spray comprising about 0.05 mL to about 0.15 mL and delivering about 0.1 mg to about 80 mg pitolisant per dose.

In some embodiments, the invention provides a method of treating one or more diseases from group comprising of narcolepsy, excessive daytime sleepiness (EDS), EDS associated with obstructive sleep apnea, idiopathic hypersomnia, Prader-Willi Syndrome, Parkinson's disease, dementia with Lewy bodies, vascular dementia, Alzheimer's disease, Attention-Deficit/Hyperactivity Disorder (ADHD), schizophrenia, depression, cognitive impairment, myotonic dystrophy, Dravet syndrome, Lennox-Gastaut syndrome, Rett syndrome, Tourette syndrome, and binge eating disorder, by administering the pitolisant liquid formulation of the present invention to the patients. In some embodiments of the present invention, the pitolisant liquid formulation of the present invention is administered to the patient at a dosing frequency of once a day, optionally twice a day, optionally twice a day at least about 2 hours apart, optionally thrice a day, optionally thrice a day at least about 2 hours apart, optionally once a week, optionally twice a week, optionally every other day, optionally once a month, optionally twice a month, optionally once a day every week, optionally once a day every other week, optionally once a day every month, optionally as needed, optionally after the onset of symptoms of the disease treatable by pitolisant, optionally prior to onset of the symptoms of the disease treatable by pitolisant (as prophylaxis), or any other dosing frequency suitable for the patient need.

In some embodiments, the present invention provides the method of use of pitolisant liquid formulation comprising steps of: (i) opening the child resistant cap of the bottle; (ii) drawing the required dose into a graduated syringe; (iii) dispensing the drawn liquid formulation in oral cavity; and (iv) optionally drinking water to rinse the oral cavity.

In some other embodiments, the present invention provides the method of use of pitolisant liquid formulation comprising steps of: (i) opening the child resistant cap of the bottle; (ii) dispensing the required dose into a measuring cup or measuring spoon or equivalent device; (iii) drinking the drawn liquid formulation via mouth; and (vi) optionally drinking the water to rinse the oral cavity.

In yet other embodiments, the present invention provides the method of use of pitolisant liquid formulation comprising steps of: (i) opening the child resistant cap of the bottle; (ii) drawing the required dose into a graduated syringe; (iii) connecting the syringe with delivery tube (such as gastric tube, naso-gastric tube, jejunum tube, etc.); (vi) pressing the piston of syringe to deliver the liquid formulation; (v) optionally flushing the delivery tube with water.

In some embodiments, the present invention relates to the method of use of pitolisant liquid formulation comprising steps of: (i) placing the actuator in nasal cavity or nostril; and (iii) actuating the device to deliver the spray of liquid formulation in nasal cavity or on nasal mucosa.

In some embodiments, either the intranasal spray or oral liquid formulation of the present invention may be administered to accommodate patients with difficulty swallowing traditional tablets or those who require medication intake without immediate access to water.

In some embodiments, the pitolisant liquid formulation of the present invention may be administered to patients across various age groups, including pediatric, adolescent, adult, and geriatric populations. Dosing and frequency may be adjusted according to the age, weight, and clinical condition of the patient to ensure safety and efficacy. Consideration of age-related factors, such as swallowing ability and metabolic rate, enables personalized treatment adapted to the needs of each patient group.

In further embodiments the pitolisant liquid formulation of the present invention may be co-administered with other medications as part of a combination therapy regimen, provided such combinations are compatible and do not adversely affect the pharmacological activity of pitolisant. In some cases, the formulation dose may be titrated according to the patient's response or therapeutic goals, with dose adjustments made under medical supervision to optimize efficacy while minimizing potential side effects.

In some embodiments, administration of pitolisant liquid formulation of the present invention may be tailored to specific times of day to coincide with the patient's symptom patterns or daily routine, thereby enhancing treatment outcomes and patient satisfaction.

In some embodiments of the present invention, the liquid formulation of pitolisant is administered to the patient for prophylaxis and/or treatment of disease treatable by pitolisant or its pharmaceutically acceptable salt such as pitolisant hydrochloride. In some embodiments of the present invention, the liquid formulation of pitolisant is administered to the patient suffering from excessive daytime sleepiness (EDS), Prader Willi syndrome (PWS), catalepsy, narcolepsy, Alzheimer's disease, schizophrenia, and/or attention-deficit/hyperactivity disorder (ADHD).

In some embodiments of the present invention, the liquid formulation of pitolisant is administered to the patient in need thereof via oral route of administration (as oral liquid solution) in volume ranging from about 0.5 mL to about 40 mL per dose. Table 1 illustrates oral liquid formulation of pitolisant hydrochloride with different concentration and specific volume of dose to be delivered respectively. It should be noted that any combination of dose, volume, or concentration can be chosen from the provided range of the present invention. For instance, a 25 mg dose at 1.25 mg/mL pitolisant hydrochloride may be administered using a 20 mL volume of liquid formulation orally. This example remains within the range indicated in the table below.

TABLE 1
Examples for use of present invention via oral administration

| Volume | | | | | | | | | | | |
|---|---|---|---|---|---|---|---|---|---|---|---|
| to be | Pitolisant Hydrochloride Concentration in mg/mL (Single Oral liquid dose) | | | | | | | | | | |
| delivered | 2.5 mg | 5 mg | 10 mg | 20 mg | 30 mg | 40 mg | 60 mg | 80 mg | 120 mg | 160 mg | 180 mg |
| per dose | dose | dose | dose | dose | dose | dose | dose | dose | dose | dose | dose |
| 0.5 mL | 5 | 10 | 20 | 40 | 60 | 80 | 120 | 160 | 240 | 320 | 360 |
| 1.0 mL | 3 | 5 | 10 | 20 | 30 | 40 | 60 | 80 | 120 | 160 | 180 |
| 2.0 mL | 1.3 | 2.5 | 5 | 10 | 15 | 20 | 30 | 40 | 60 | 80 | 90 |
| 2.5 mL | 1 | 2 | 4 | 8 | 12 | 16 | 24 | 32 | 48 | 64 | 72 |
| 4.0 mL | 0.63 | 1.25 | 2.5 | 5 | 7.5 | 10 | 15 | 20 | 30 | 40 | 45 |
| 5.0 mL | 1 | 1 | 2 | 4 | 6 | 8.0 | 12 | 16 | 24 | 32 | 36 |
| 8.0 mL | 0.3125 | 0.625 | 1.25 | 2.5 | 3.75 | 5 | 7.5 | 10 | 15 | 20 | 22.5 |
| 10.0 mL | 0.3 | 0.5 | 1 | 2 | 3 | 4 | 6 | 8 | 12 | 16 | 18 |
| 40.0 mL | 0.0625 | 0.125 | 0.25 | 0.5 | 0.75 | 1.0 | 1.5 | 2 | 3 | 4 | 4.5 |

In some embodiments of the present invention, the liquid formulation of pitolisant is administered to the patient in need thereof via oral route of administration (as oral spray solution) in volume ranging from about 0.050 mL per spray to about 0.250 mL per spray depending on target dose for delivery and concentration of pitolisant in formulation (refer to table 2). In some other embodiments, the liquid formulation of pitolisant is administered to the patient in need thereof via oral route of administration (as oral spray solution) from about 0.05 mL to about 1 mL in total delivered volume administered as more than one spray per dose. Table 3 illustrates concentration of pitolisant, and volume of each spray delivered for target dose as two spray therapy per dose. It is understood that any combination of dose, volume, or concentration may be selected from the given range or even at higher doses with extrapolation based on patient needs. For instance, a 15 mg dose at 150 mg/mL pitolisant hydrochloride can be administered orally using 0.1 mL of formulation delivered as a single spray. This example falls within the scope of the invention. Likewise, multiple sprays may be utilized to achieve the desired dose by adjusting the concentration and/or the delivery volume.

TABLE 2
Examples for use of present invention via oral administration

| Volume | | | | | | | | | | | |
|---|---|---|---|---|---|---|---|---|---|---|---|
| to be | Pitolisant Hydrochloride Concentration in mg/mL (Single Oral Spray per Dose) | | | | | | | | | | |
| delivered | 2.5 mg | 5 mg | 10 mg | 20 mg | 30 mg | 40 mg | 60 mg | 80 mg | 120 mg | 160 mg | 180 mg |
| per Spray | dose | dose | dose | dose | dose | dose | dose | dose | dose | dose | dose |
| 0.050 mL | 50.0 | 100.0 | 200 | 400 | 600 | 800 | — | — | — | — | — |
| 0.080 mL | 31.3 | 62.5 | 125 | 250 | 375 | 500 | 750 | — | — | — | — |
| 0.100 mL | 25 | 50 | 100 | 200 | 300 | 400 | 600 | 800 | — | — | — |
| 0.125 mL | 20 | 40 | 80 | 160 | 240 | 320 | 480 | 640 | 960 | — | — |
| 0.160 mL | 15.625 | 31.25 | 62.5 | 125 | 187.5 | 250 | 375 | 500 | 750 | 1000 | — |
| 0.200 mL | 13 | 25 | 50 | 100 | 150 | 200 | 300 | 400 | 600 | 800 | 900 |
| 0.250 mL | 10 | 20 | 40 | 80 | 120 | 160 | 240 | 320 | 480 | 640 | 720 |

TABLE 3
Examples for use of present invention via oral administration

| Volume | | | | | | | | | | | |
|---|---|---|---|---|---|---|---|---|---|---|---|
| to be | Pitolisant Hydrochloride Concentration in mg/mL (Two Oral Sprays per Dose) | | | | | | | | | | |
| delivered | 2.5 mg | 5 mg | 10 mg | 20 mg | 30 mg | 40 mg | 60 mg | 80 mg | 120 mg | 160 mg | 180 mg |
| per Spray | dose | dose | dose | dose | dose | dose | dose | dose | dose | dose | dose |
| 0.050 mL | 25.0 | 50.0 | 100.0 | 200.0 | 300.0 | 400.0 | 600.0 | 800.0 | — | — | — |
| 0.080 mL | 15.63 | 31.25 | 62.5 | 125.0 | 187.5 | 250.0 | 375.0 | 500.0 | 750.0 | 1000.0 | — |
| 0.100 mL | 12.5 | 25.0 | 50.0 | 100.0 | 150.0 | 200.0 | 300.0 | 400.0 | 600.0 | 800.0 | 900.0 |
| 0.125 mL | 10.0 | 20.0 | 40.0 | 80.0 | 120.0 | 160.0 | 240.0 | 320.0 | 480.0 | 640.0 | 720.0 |
| 0.160 mL | 7.8 | 15.6 | 31.25 | 62.5 | 93.75 | 125.0 | 187.5 | 250.0 | 375.0 | 500.0 | 562.5 |
| 0.200 mL | 6.3 | 12.5 | 25.0 | 50.0 | 75.0 | 100.0 | 150.0 | 200.0 | 300.0 | 400.0 | 450.0 |
| 0.250 mL | 5.0 | 10.0 | 20.0 | 40.0 | 60.0 | 80.0 | 120.0 | 160.0 | 240.0 | 320.0 | 360 |

The following tables illustrate nasal spray formulation of pitolisant hydrochloride with different concentration and specific volume of dose to be delivered respectively. It is understood that any combination of dose, volume, or concentration may be selected from the given range or even at higher doses with extrapolation based on patient needs. For instance, a 15 mg dose at 150 mg/mL pitolisant hydrochloride can be delivered using a 0.1 mL volume of formulation administered intranasally as a single spray. This example falls within the range provided in the table below and scope of the present invention.

TABLE 4
Examples for use of present invention via intranasal administration

| Volume | | | | | | | | | | | |
|---|---|---|---|---|---|---|---|---|---|---|---|
| to be | Pitolisant Hydrochloride Concentration in mg/mL (Single Nasal Spray per Dose) | | | | | | | | | | |
| delivered | 2.5 mg | 5 mg | 10 mg | 20 mg | 30 mg | 40 mg | 60 mg | 80 mg | 120 mg | 160 mg | 180 mg |
| per Spray | dose | dose | dose | dose | dose | dose | dose | dose | dose | dose | dose |
| 0.050 mL | 50 | 100 | 200 | 400 | 600 | 800 | 1200 | — | — | — | — |
| 0.080 mL | 31.3 | 62.5 | 125.0 | 250.0 | 375.0 | 500.0 | 750.0 | 1000.0 | — | — | — |
| 0.100 mL | 25 | 50 | 100 | 200 | 300 | 400 | 600 | 800 | — | — | — |
| 0.125 mL | 20 | 40 | 80 | 160 | 240 | 320 | 480 | 640 | 960 | — | — |

TABLE 5
Examples for use of present invention via intranasal administration

| Volume to | Pitolisant Hydrochloride Concentration in mg/mL (Two Nasal Sprays per Dose) | | | | | | | | | | |
|---|---|---|---|---|---|---|---|---|---|---|---|
| be delivered | 2.5 mg | 5 mg | 10 mg | 20 mg | 30 mg | 40 mg | 60 mg | 80 mg | 120 mg | 160 mg | 180 mg |
| per Spray | dose | dose | dose | dose | dose | dose | dose | dose | dose | dose | dose |
| 0.050 mL | 25 | 50 | 100 | 200 | 300 | 400 | 600 | 800 | — | — | — |
| 0.080 mL | 16 | 31.25 | 63 | 125 | 188 | 250 | 375 | 500 | 750 | 1000 | — |
| 0.100 mL | 12.5 | 25 | 50 | 100 | 150 | 200 | 300 | 400 | 600 | 800 | 900 |
| 0.125 mL | 10 | 20 | 40 | 80 | 120 | 160 | 240 | 320 | 480 | 640 | 960 |

The following tables illustrate various doses and concentration of pitolisant, or its pharmaceutically acceptable salt (such as hydrochloride salt) provided as liquid formulation filled in soft or hard gelatin capsule. It is understood that any combination of dose, volume, or concentration may be selected from the given range or even at higher doses with extrapolation based on patient needs.

TABLE 6
Examples for use of present invention via oral administration

| Soft | | Pitolisant approximate concentration in mg/mL (One soft gelatin delayed | | | | | | | | | | |
|---|---|---|---|---|---|---|---|---|---|---|---|---|
| Gelatin | | release capsule) | | | | | | | | | | |
| Capsule | Fill | 2.5 | 5 | 10 | 20 | 30 | 40 | 60 | 80 | 120 | 160 | 180 |
| Size | Volume | mg | mg | mg | mg | mg | mg | mg | mg | mg | mg | mg |
| (Minims) | (mL) | dose | dose | dose | dose | dose | dose | dose | dose | dose | dose | dose |
| 2 | ~0.12 | 20.8 | 41.7 | 83.3 | 166.7 | 250.0 | 333.3 | 500.0 | 666.7 | 1000 | — | — |
| 2 | ~0.12 | 20.8 | 41.7 | 83.3 | 166.7 | 250.0 | 333.3 | 500.0 | 666.7 | 1000 | — | — |
| 3 | ~0.19 | 13.2 | 26.3 | 52.6 | 105.3 | 157.9 | 210.5 | 315.8 | 421.1 | 631.6 | 842.1 | 947.4 |
| 3 | ~0.19 | 13.2 | 26.3 | 52.6 | 105.3 | 157.9 | 210.5 | 315.8 | 421.1 | 631.6 | 842.1 | 947.4 |
| 4 | ~0.25 | 10.0 | 20.0 | 40.0 | 80.0 | 120.0 | 160.0 | 240.0 | 320.0 | 480.0 | 640 | 720 |
| 4 | ~0.25 | 10.0 | 20.0 | 40.0 | 80.0 | 120.0 | 160.0 | 240.0 | 320.0 | 480.0 | 640 | 720 |
| 5 | ~0.31 | 8.1 | 16.1 | 32.3 | 64.5 | 96.8 | 129.0 | 193.5 | 258.1 | 387.1 | 516.1 | 580.6 |
| 6 | ~0.37 | 6.8 | 13.5 | 27.0 | 54.1 | 81.1 | 108.1 | 162.2 | 216.2 | 324.3 | 432.4 | 486.5 |
| 8 | ~0.50 | 5.0 | 10.0 | 20.0 | 40.0 | 60.0 | 80.0 | 120.0 | 160.0 | 240.0 | 320.0 | 360.0 |
| 9 | ~0.56 | 4.5 | 8.9 | 17.9 | 35.7 | 53.6 | 71.4 | 107.1 | 142.9 | 214.3 | 285.7 | 321.4 |
| 10 | ~0.62 | 4.0 | 8.1 | 16.1 | 32.3 | 48.4 | 64.5 | 96.8 | 129.0 | 193.5 | 258.1 | 290.3 |
| 12 | ~0.74 | 3.4 | 6.8 | 13.5 | 27.0 | 40.5 | 54.1 | 81.1 | 108.1 | 162.2 | 216.2 | 243.2 |
| 14 | ~0.86 | 2.9 | 5.8 | 11.6 | 23.3 | 34.9 | 46.5 | 69.8 | 93.0 | 139.5 | 186.0 | 209.3 |
| 16 | ~1.00 | 2.5 | 5.0 | 10.0 | 20.0 | 30.0 | 40.0 | 60.0 | 80.0 | 120.0 | 160.0 | 180.0 |
| 16 | ~1.00 | 2.5 | 5.0 | 10.0 | 20.0 | 30.0 | 40.0 | 60.0 | 80.0 | 120.0 | 160.0 | 180.0 |
| 20 | ~1.23 | 2.0 | 4.1 | 8.1 | 16.3 | 24.4 | 32.5 | 48.8 | 65.0 | 97.6 | 130.1 | 146.3 |
| 22 | ~1.36 | 1.8 | 3.7 | 7.4 | 14.7 | 22.1 | 29.4 | 44.1 | 58.8 | 88.2 | 117.6 | 132.4 |
| 24 | ~1.48 | 1.7 | 3.4 | 6.8 | 13.5 | 20.3 | 27.0 | 40.5 | 54.1 | 81.1 | 108.1 | 121.6 |

TABLE 7
Examples for use of present invention via oral administration

| | | Pitolisant approximate Concentration in mg/mL (One hard gelatin delayed | | | | | | | | | | |
|---|---|---|---|---|---|---|---|---|---|---|---|---|
| | | release capsule) | | | | | | | | | | |
| | Fill | 2.5 | 5 | 10 | 20 | 30 | 40 | 60 | 80 | 120 | 160 | 180 |
| Capsule | Volume | mg | mg | mg | mg | mg | mg | mg | mg | mg | mg | mg |
| Size | (mL) | dose | dose | dose | dose | dose | dose | dose | dose | dose | dose | dose |
| 5 | ~0.13 | 19.2 | 38.5 | 76.9 | 153.8 | 230.8 | 307.7 | 461.5 | 615.4 | 923.1 | — | — |
| 4 | ~0.21 | 11.9 | 23.8 | 47.6 | 95.2 | 142.9 | 190.5 | 285.7 | 381.0 | 571.4 | 761.9 | 857.1 |
| 3 | ~0.30 | 8.3 | 16.7 | 33.3 | 66.7 | 100.0 | 133.3 | 200.0 | 266.7 | 400.0 | 533.3 | 600.0 |
| 2 | ~0.37 | 6.8 | 13.5 | 27.0 | 54.1 | 81.1 | 108.1 | 162.2 | 216.2 | 324.3 | 432.4 | 486.5 |
| 1 | ~0.50 | 5.0 | 10.0 | 20.0 | 40.0 | 60.0 | 80.0 | 120.0 | 160.0 | 240.0 | 320.0 | 360.0 |
| 0 | ~0.68 | 3.7 | 7.4 | 14.7 | 29.4 | 44.1 | 58.8 | 88.2 | 117.6 | 176.5 | 235.3 | 264.7 |
| 00 | ~0.95 | 2.6 | 5.3 | 10.5 | 21.1 | 31.6 | 42.1 | 63.2 | 84.2 | 126.3 | 168.4 | 189.5 |
| 000 | ~1.37 | 1.8 | 3.6 | 7.3 | 14.6 | 21.9 | 29.2 | 43.8 | 58.4 | 87.6 | 116.8 | 131.4 |

In some embodiments, the preferred ingredient for delayed release oral formulation is pitolisant hydrochloride salt.

In some embodiments, the invention provides a pharmaceutical composition for intranasal delivery comprising: a therapeutically effective amount of pitolisant, or a pharmaceutically acceptable salt thereof, at a concentration between 5 mg/mL and 400 mg/mL; at least one pharmaceutically acceptable viscosity-enhancing agent at a concentration between about 0.1% and about 5.0% (w/v); and an aqueous carrier. The viscosity-enhancing agent is selected from the group consisting of hypromellose (HPMC), carboxymethyl cellulose (CMC), sodium alginate, carbomers, xanthan gum, and carrageenan, such that the final formulation has a viscosity at room temperature between about 5 and about 2000 centipoise (cP). In another embodiment, the viscous nasal spray formulation is administered to a subject, resulting in a unique pharmacokinetic profile characterized by a mean time to maximum plasma concentration (Tₘₐₓ) between about 60 and about 300 minutes. This Tₘₐₓ is substantially longer than that achievable by a non-viscous intranasal spray and is designed to mimic the absorption rate of an oral solid dose. However, by avoiding gastrointestinal transit and first-pass metabolism, the formulation provides greater bioavailability and more predictable absorption than an oral tablet, while the controlled Cₘₐₓ mitigates centrally-mediated nausea. In a further embodiment, the composition is enhanced by the inclusion of a mucoadhesive polymer. Said polymer, which may be chitosan, a polyacrylic acid derivative (e.g., Carbopol® 974P), or a thiolated polymer, constitutes between about 0.05% and about 2.0% (w/v) of the formulation. The inclusion of the mucoadhesive agent enhances the adhesion of the viscous formulation to the nasal epithelium, preventing premature clearance by mucociliary action and ensuring a more complete and prolonged drug release, leading to improved dose consistency.

In yet another embodiment, the invention is an in-situ gelling liquid formulation for intranasal administration. The formulation is a low-viscosity liquid at storage temperature (e.g., 2-8° C. or about 15-25° C.) for ease of atomization from a spray device but rapidly transforms into a high-viscosity gel upon contact with the nasal mucosa at body temperature (about 34 to about 37° C.). This is achieved using a thermo-responsive polymer, such as a poloxamer (e.g., Poloxamer 407), or an ion-sensitive polymer, such as gellan gum, that gels in the presence of nasal cations. This embodiment provides the benefits of a controlled-release depot while maintaining the convenience and accuracy of a standard liquid nasal spray device.

In another embodiment, the invention provides a method for treating excessive daytime sleepiness or cataplexy in a human subject. The method comprises, first, identifying a subject who has demonstrated intolerance to oral pitolisant, characterized by adverse events including nausea, dyspepsia, or stomach pain. Second, the method involves administering to said subject the viscous intranasal pitolisant formulation described herein. The administration of said intranasal formulation results in a sustained therapeutic effect equivalent to an oral dose but with a statistically significant reduction in the frequency and severity of the previously experienced adverse events.

### Working Examples

The present invention is further illustrated by, but is by no means limited to, the following examples. It will be appreciated that where typical or preferred process conditions are given, other process conditions can also be used unless otherwise stated. Materials used in the examples set forth below were provided by commercial suppliers. It is preferred and recommended that ingredients, used in pitolisant liquid formulations, comply with the compendial monograph such as USP, Ph. Eur., and/or JP, and ICH guidelines for residual process related impurities (elemental impurities and residual solvents).

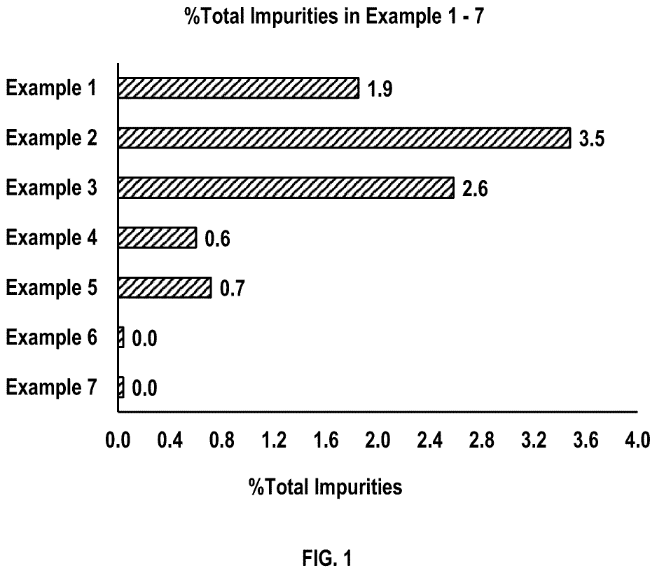

Pitolisant is reported as sensitive to moisture and oxidative condition in prior art of a solid dosage form. Therefore, series of experiments were conducted to understand stability behavior of pitolisant in liquid carrier. Compositions of example 1-7 are provided in Table 8. Example 1 was prepared at ambient condition by transferring 20 mg pitolisant hydrochloride crystalline powder alone in the vial without any other ingredient. Example 2-7 were prepared at ambient condition by dissolving pitolisant hydrochloride with other ingredients in water. All samples were filled in 2R USP Type I clear tubular glass vial, stoppered without nitrogen overlay, crimp sealed and subjected to heat treatment of 121° C. for 15 minutes as accelerated condition in autoclave. It was observed that pitolisant hydrochloride crystalline powder turned into a liquid solution during treatment (possibly due to moisture present in sealed vial, weight was verified for samples to assure no-ingress of water during autoclave) and solidified upon cooling to room temperature. After treatment, total impurities for these samples are tested using HPLC and result presented in FIG. 1. It was surprisingly found that pitolisant hydrochloride drug substance without any other ingredient (example 1) showed unexpectedly higher impurities compared to sample prepared in water (example 7). Although sodium metabisulfite was added to stabilize the drug, it unexpectedly caused degradation of pitolisant. The degradation caused by sodium metabisulfite was inversely proportional to pitolisant concentration (example 2-4). Composition containing ascorbic acid (example 5) also showed slight higher impurities than without ascorbic acid in water. Samples prepared in water with and without disodium edetate had comparable % total impurities.

TABLE 8
Composition of example 1-7

| | Example | Example | Example | Example | Example | Example | Example |
|---|---|---|---|---|---|---|---|
| Ingredients | 1* | 2 | 3 | 4 | 5 | 6 | 7 |
| Pitolisant | 20 mg | 8 mg | 20 mg | 200 mg | 20 mg | 20 mg | 20 mg |
| hydrochloride | | | | | | | |
| Sodium | — | 2 mg | 2 mg | 2 mg | — | — | — |
| metabisulfite | | | | | | | |
| Ascorbic acid | — | — | — | — | 2 mg | — | — |
| Disodium edetate | — | — | — | — | — | 2 mg | — |
| dihydrate | | | | | | | |
| Purified water | — | q.s. to 2 | q.s. to 2 | q.s. to 2 | q.s. to 2 | q.s. to 2 | q.s. to 2 |
| | | mL | mL | mL | mL | mL | mL |
| Fill volume | — | 2 mL | 2 mL | 2 mL | 2 mL | 2 mL | 2 mL |

*Pitolisant hydrochloride crystalline powder without any vehicle or excipient.

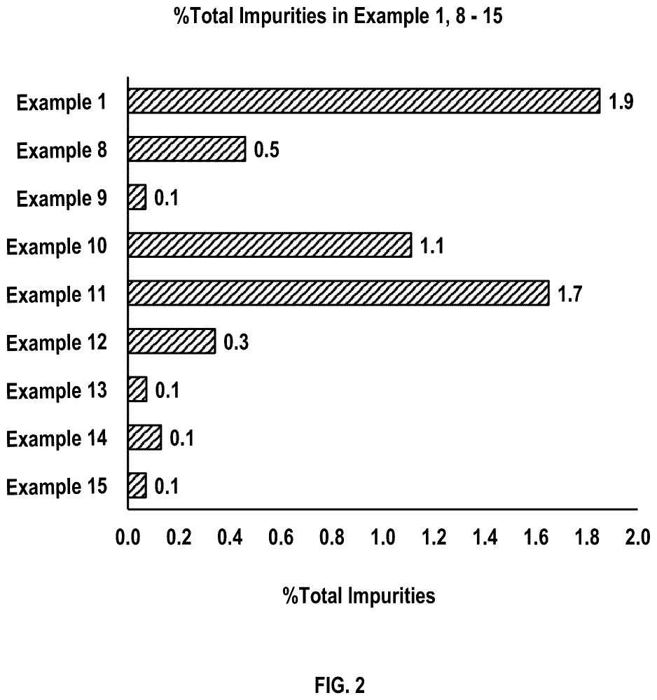

Example 8-15 were prepared using compositions provided in Table 9, by dissolving, where applicable, pitolisant hydrochloride, and tocopherol in required quantity of polyethylene glycol 400 (example 8-10, 14-15), water soluble ingredients (disodium edetate, sodium metabisulfite, and ascorbic acid) in about 80% of required quantity of water followed by adding water solution to PEG solution (example 11-13), and making up the volume using purified water (example 10-13, 15). All samples were filled in 2R USP Type I clear tubular glass vial, stoppered without nitrogen overlay (except example 9), crimp sealed and subjected to heat treatment of 121° C. for 15 minutes as accelerated condition in autoclave. After treatment, total impurities for these samples were tested using HPLC and result presented in FIG. 2.

TABLE 9
Composition of example 8-15.

| | Example | Example | Example | Example | Example | Example | Example | Example |
|---|---|---|---|---|---|---|---|---|
| Ingredients | 8 | 9* | 10 | 11 | 12 | 13 | 14 | 15 |
| Pitolisant | 20 mg | 20 mg | 20 mg | 20 mg | 20 mg | 20 mg | 20 mg | 20 mg |
| hydrochloride | | | | | | | | |
| Disodium | — | — | — | 2 mg | — | — | — | — |
| edetate | | | | | | | | |
| dihydrate | | | | | | | | |
| Sodium | — | — | — | — | 2 mg | — | — | — |
| metabisulfite | | | | | | | | |
| Ascorbic acid | — | — | — | — | — | 2 mg | — | — |
| All-rac-α- | — | — | — | — | — | — | 2 mg | 2 mg |
| tocopherol | | | | | | | | |
| Polyethylene | q.s. to 2 | q.s. to 2 | 1.6 mL | 1.6 mL | 1.6 mL | 1.6 mL | q.s. to 2 | 1.6 mL |
| glycol 400 | mL | mL | | | | | mL | |
| Purified water | — | — | q.s. to 2 | q.s. to 2 | q.s. to 2 | q.s. to 2 | — | q.s. to 2 |
| | | | mL | mL | mL | mL | | mL |
| Fill volume | 2 mL | 2 mL | 2 mL | 2 mL | 2 mL | 2 mL | 2 mL | 2 mL |

*Nitrogen overlay

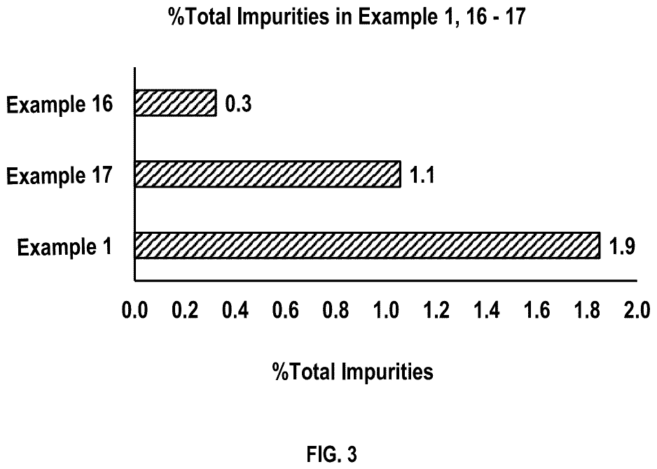

Examples 16 and 17 were prepared using compositions provided in Table 10, by transferring required amount of pitolisant hydrochloride crystalline powder with, where applicable, disodium edetate or citric acid without use of any solvent. Both samples were filled in 2R USP Type I clear tubular glass vial, stoppered without nitrogen overlay, crimp sealed and subjected to heat treatment of 121° C. for 15 minutes as accelerated condition in autoclave. It was observed that the mixture turned into a liquid solution during treatment, possibly due to moisture present in sealed vial, and solidified upon cooling to room temperature. After treatment, total impurities were tested using HPLC and result presented in FIG. 3.

TABLE 10
Composition of example 16-17

| Ingredients | Example 16 | Example 17 |
|---|---|---|
| Pitolisant hydrochloride | 20 mg | 20 mg |
| Disodium edetate dihydrate | 1 mg | — |
| Citric acid monohydrate | — | 2 mg |

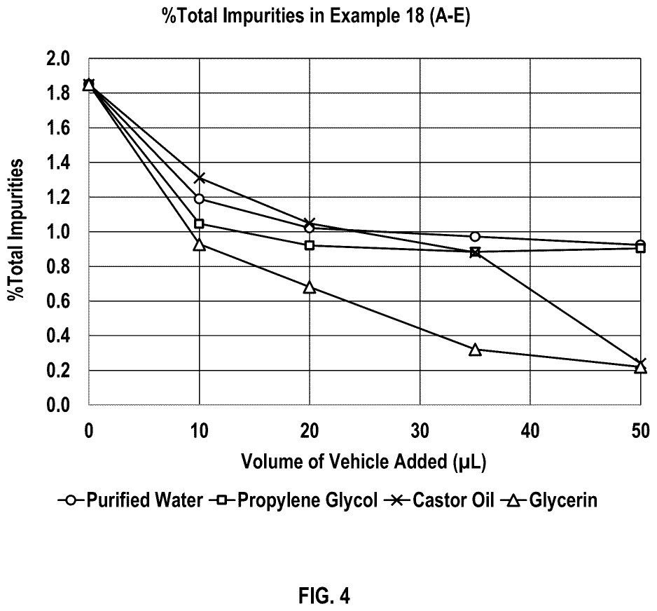

Samples for Examples 18 (A-E) were prepared using compositions provided in Table 11, by transferring required amount of pitolisant hydrochloride crystalline powder with, where applicable, vehicle at amount described in Table 11 into 2R USP Type I clear tubular glass vial, stoppered without nitrogen overlay, crimp sealed and subjected to heat treatment of 121° C. for 15 minutes as accelerated condition in autoclave. After treatment, total impurities were tested using HPLC and result presented in FIG. 4.

TABLE 11
Composition of example 18(A-E).

| | Pitolisant | Purified | Propylene | Castor | |
|---|---|---|---|---|---|
| Example# | hydrochloride | Water | glycol | oil | Glycerin |
| 18A | 50 mg | — | — | — | — |
| 18B1 | 50 mg | 10 μL | — | — | — |
| 18B2 | 50 mg | 20 μL | — | — | — |
| 18B3 | 50 mg | 35 μL | — | — | — |
| 18B4 | 50 mg | 50 μL | — | — | — |
| 18C1 | 50 mg | — | 10 μL | — | — |
| 18C2 | 50 mg | — | 20 μL | — | — |
| 18C3 | 50 mg | — | 35 μL | — | — |
| 18C4 | 50 mg | — | 50 μL | — | — |
| 18D1 | 50 mg | — | — | 10 μL | — |
| 18D2 | 50 mg | — | — | 20 μL | — |
| 18D3 | 50 mg | — | — | 35 μL | — |
| 18D4 | 50 mg | — | — | 50 μL | — |
| 18E1 | 50 mg | — | — | — | 10 μL |
| 18E2 | 50 mg | — | — | — | 20 μL |
| 18E3 | 50 mg | — | — | — | 35 μL |
| 18E4 | 50 mg | — | — | — | 50 μL |

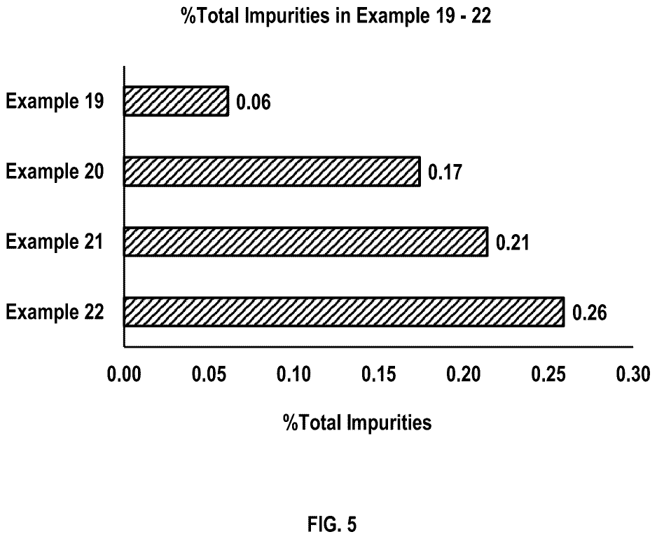

Samples for Examples 19-22 were prepared using compositions provided in Table 12, by dissolving required quantity of pitolisant hydrochloride in water, filled into 2R USP Type I clear tubular glass vial, stoppered without nitrogen overlay, crimp sealed and subjected to heat treatment of 121° C. for 15 minutes as accelerated condition in autoclave. After treatment, total impurities were tested using HPLC and result presented in FIG. 5.

TABLE 12
Composition of example 19-22

| Ingredients | Example 19 | Example 20 | Example 21 | Example 22 |
|---|---|---|---|---|
| Pitolisant | 100 mg | 200 mg | 300 mg | 400 mg |
| hydrochloride | | | | |
| Purified water | q.s. to 1 mL | q.s. to 1 mL | q.s. to 1 mL | q.s. to 1 mL |
| Fill volume | 0.5 mL | 0.5 mL | 0.5 mL | 0.5 mL |

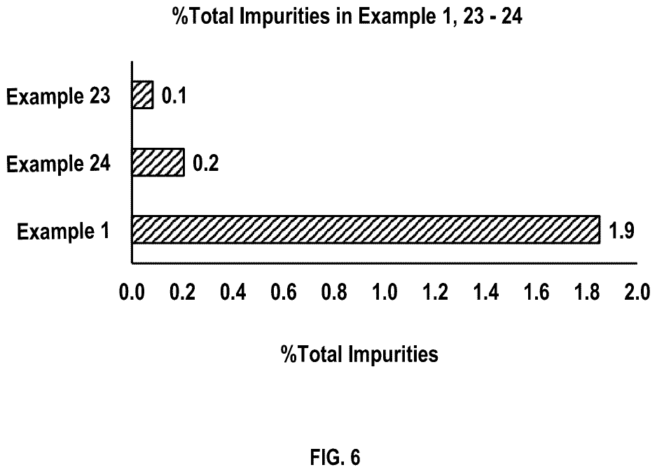

Sample for Examples 23 and 24 were prepared using compositions provided in Table 13. Example 23 was prepared by dissolving required quantity of pitolisant hydrochloride in castor oil. Example 24 was prepared using pitolisant free base, that was prepared from pitolisant hydrochloride salt using 1N sodium hydroxide followed by washing with water, separation and dissolving into sesame oil. Pitolisant free base may be used directly, if available. The samples were filled into 2R USP Type I clear tubular glass vial, stoppered with nitrogen overlay, crimp sealed and subjected to heat treatment of 121° C. for 15 minutes as accelerated condition in autoclave. After treatment, total impurities for these samples were tested using HPLC and results provided in FIG. 6.

TABLE 13
Composition of example 23-24

| | | |
|---|---|---|
| Ingredients | Example 23 | Example 24 |

| | | | |
|---|---|---|---|
| Pitolisant hydrochloride | 20 | mg | — |

| | | | |
|---|---|---|---|
| Pitolisant free base | — | ~20 | mg |

| | | |
|---|---|---|
| Castor oil | q.s. to 2 mL | — |
| Sesame oil | — | q.s. to 2 mL |

| | | | | |
|---|---|---|---|---|
| Fill volume | 2 | mL | 2 | mL |

Examples 25-28 describe the liquid formulation of pitolisant hydrochloride, prepared using ingredients detailed in Table 14. Where applicable, the preservatives (methylparaben and propylparaben) were added one-by-one to approximately 80% of the total required quantity of purified water, which was optionally heated to 50-60° C. to aid dissolution of preservatives. After complete dissolution, this solution was cooled to room temperature (20-25° C.). Where applicable, sucralose, strawberry flavor, citric acid monohydrate, and tri-sodium citrate were added sequentially and mixed until dissolved. The active ingredient, pitolisant hydrochloride, was then added and mixed until it dissolved. Finally, the solution's volume was brought up to the batch size with purified water and mixed to homogeneity. The formulation was then filled into containers, and sealed with closure with nitrogen overlay. Stability test results for example 25-28 are provided in Table 15.

TABLE 14
Compositions for pitolisant liquid formulation of example 25-28.

| | Example 25 | Example 26 | Example 27 | Example 28 |
|---|---|---|---|---|
| Ingredients | (% w/v) | (% w/v) | (% w/v) | (% w/v) |
| Pitolisant hydrochloride | 0.5% | 0.5% | 0.5% | 1% |
| Sucralose | — | 0.5% | — | 0.5% |
| Methylparaben | — | 0.2% | — | 0.25 |
| Propylparaben | — | 0.02% | — | 0.025% |
| Strawberry flavor | — | 0.1% | — | 0.1% |
| Citric acid monohydrate | — | — | 0.2% | 0.2% |
| Tri-sodium citrate dihydrate | — | — | 0.05% | 0.05% |
| Purified Water | q.s. to 100% | q.s. to 100% | q.s. to 100% | q.s. to 100% |

TABLE 15
Stability test results for example 25-28.

| | | | | | % | | |
|---|---|---|---|---|---|---|---|
| | | | | | Maximum | | |
| Example | Time | | % | % PTS | Single | % Total | |
| No. | point | Visual Description | Purity | N-Oxide | Impurity | Impurities | pH |
| 25 | Initial | Clear, colorless solution | 99.9 | <0.05 | 0.03 | 0.1 | 5.3 |
| | 40° C./1 M | Clear, colorless solution | 100.0 | <0.05 | 0.03 | 0.1 | — |
| | 40° C./2 M | Clear, colorless solution | 99.9 | <0.05 | 0.05 | 0.1 | — |
| | 40° C./3 M | Clear, colorless solution | 99.9 | <0.05 | 0.04 | 0.1 | — |
| | 40° C./6 M | Clear, colorless solution | 99.9 | <0.05 | 0.03 | 0.1 | 5.7 |
| | RT/1 M | Clear, colorless solution | 100.0 | <0.05 | 0.03 | 0.0 | — |
| | RT/3 M | Clear, colorless solution | 99.9 | <0.05 | 0.04 | 0.1 | — |
| | RT/6 M | Clear, colorless solution | 99.9 | <0.05 | 0.03 | 0.1 | — |
| | RT/9 M | Clear, colorless solution | 99.9 | <0.05 | 0.04 | 0.1 | 5.4 |
| 26 | Initial | Clear, colorless solution | 99.9 | <0.05 | 0.03 | 0.1 | 4.6 |
| | 40° C./1 M | Clear, colorless solution | 100.0 | <0.05 | 0.03 | 0.1 | — |
| | 40° C./2 M | Clear, colorless solution | 99.9 | <0.05 | 0.04 | 0.1 | — |
| | 40° C./3 M | Clear, colorless solution | 99.9 | <0.05 | 0.04 | 0.1 | — |
| | 40° C./6 M | Clear, colorless solution | 99.9 | <0.05 | 0.03 | 0.1 | 4.4 |
| | RT/1 M | Clear, colorless solution | 99.9 | <0.05 | 0.03 | 0.1 | — |
| | RT/3 M | Clear, colorless solution | 99.9 | <0.05 | 0.04 | 0.1 | — |
| | RT/6 M | Clear, colorless solution | 99.9 | <0.05 | 0.04 | 0.1 | — |
| | RT/9 M | Clear, colorless solution | 99.9 | <0.05 | 0.04 | 0.1 | 4.5 |
| 27 | Initial | Clear, colorless solution | 99.9 | <0.05 | 0.04 | 0.1 | 3.2 |
| | 40° C./1 M | Clear, colorless solution | 99.8 | <0.05 | 0.14 | 0.2 | — |
| | 40° C./2 M | Clear, colorless solution | 99.9 | <0.05 | 0.15 | 0.2 | — |
| | 40° C./3 M | Clear, colorless solution | 99.7 | <0.05 | 0.09 | 0.3 | — |
| | 40° C./6 M | Clear, colorless solution | 99.8 | <0.05 | 0.06 | 0.2 | 3.2 |
| | RT/1 M | Clear, colorless solution | 99.9 | <0.05 | 0.03 | 0.1 | — |
| | RT/3 M | Clear, colorless solution | 99.9 | <0.05 | 0.05 | 0.1 | — |
| | RT/6 M | Clear, colorless solution | 99.9 | <0.05 | 0.04 | 0.1 | — |
| | RT/9 M | Clear, colorless solution | 99.9 | <0.05 | 0.04 | 0.1 | 3.2 |
| 28 | Initial | Clear, colorless solution | 99.9 | <0.05 | 0.04 | 0.1 | 3.1 |
| | 60° C./2 W | Clear, colorless solution | 100.0 | <0.05 | 0.03 | 0.1 | — |
| | 60° C./1 M | Clear, colorless solution | 99.0 | <0.05 | 0.96 | 1.1 | — |
| | 40° C./1 M | Clear, colorless solution | 100.0 | <0.05 | 0.03 | 0.1 | — |
| | 40° C./2 M | Clear, colorless solution | 99.8 | <0.05 | 0.20 | 0.2 | — |
| | 40° C./3 M | Clear, colorless solution | 99.9 | <0.05 | 0.04 | 0.1 | — |
| | 40° C./6 M | Clear, colorless solution | 99.9 | <0.05 | 0.03 | 0.1 | 3.1 |
| | RT/3 M | Clear, colorless solution | 99.9 | <0.05 | 0.03 | 0.1 | — |
| | RT/6 M | Clear, colorless solution | 99.9 | <0.05 | 0.03 | 0.1 | — |
| | RT/9 M | Clear, colorless solution | 99.9 | <0.05 | 0.04 | 0.1 | 3.1 |

Examples 29-33 describe the liquid formulation of pitolisant hydrochloride, prepared using ingredients detailed in Table 16. The preservatives (methylparaben and propylparaben) were added one-by-one to approximately 80% of the total required quantity of purified water, which was optionally heated to 50-60° C. to aid dissolution. After complete dissolution, this solution was cooled to room temperature (20-25° C.). Sucralose, and strawberry flavor were added sequentially and mixed until dissolved. The active ingredient, pitolisant hydrochloride, was then added and mixed until it dissolved. pH of the solution was adjusted to target using dilute hydrochloric acid and/or sodium hydroxide. Finally, the solution's volume was brought up to the batch size with purified water and mixed to homogeneity. The formulation was filled into containers, and sealed with closure. Stability test results for example 29-33 are provided in Table 17.

TABLE 16
Compositions for pitolisant liquid formulation of example 29-33.

| | Example 29 | Example 30 | Example 31 | Example 32 | Example 33 |
|---|---|---|---|---|---|
| Ingredients | (% w/v) | (% w/v) | (% w/v) | (% w/v) | (% w/v) |
| Pitolisant hydrochloride | 0.5% | 0.5% | 0.5% | 0.5% | 0.5% |
| Sucralose | 0.5% | 0.5% | 0.5% | 0.5% | 0.5% |
| Methylparaben | 0.2% | 0.2% | 0.2% | 0.2% | 0.2% |
| Propylparaben | 0.02% | 0.02% | 0.02% | 0.02% | 0.02% |
| Strawberry flavor | 0.1% | 0.1% | 0.1% | 0.1% | 0.1% |
| Hydrochloric acid (0.1N) | q.s. to | q.s. to | q.s. to | q.s. to | q.s. to |
| | pH 3.0 | pH 3.8 | pH 3.4 | pH 2.5 | pH 5.2 |
| Sodium hydroxide (0.1N) | — | — | — | — | q.s. to |
| | | | | | pH 5.2 |
| Purified Water | q.s. to 100% | q.s. to 100% | q.s. to 100% | q.s. to 100% | q.s. to 100% |
| Final pH of Solution | 3.1 | 3.9 | 3.5 | 2.6 | 5.1 |

TABLE 17
Stability test results for example 29-33.

| | | | | | % | | |
|---|---|---|---|---|---|---|---|
| | | | | | Maximum | | |
| Example | Time | | % | % PTS | Single | % Total | pH of |
| No. | point | Visual Description | Purity | N-Oxide | Impurity | Impurities | Solution |
| 29 | Initial | Clear, colorless solution | 99.9 | <0.05 | 0.03 | 0.1 | 3.1 |
| | 40° C./1 M | Clear, colorless solution | 99.9 | <0.05 | 0.03 | 0.1 | — |
| | 40° C./2 M | Clear, colorless solution | 99.9 | <0.05 | 0.04 | 0.1 | — |
| | 40° C./3 M | Clear, colorless solution | 99.9 | <0.05 | 0.04 | 0.1 | — |
| | 40° C./6 M | Clear, colorless solution | 99.9 | <0.05 | 0.05 | 0.1 | 3.2 |
| | RT/1 M | Clear, colorless solution | 100.0 | <0.05 | 0.03 | 0.0 | — |
| | RT/3 M | Clear, colorless solution | 99.9 | <0.05 | 0.04 | 0.1 | — |
| | RT/6 M | Clear, colorless solution | 99.9 | <0.05 | 0.03 | 0.1 | — |
| | RT/9 M | Clear, colorless solution | 99.9 | <0.05 | 0.03 | 0.1 | 3.1 |
| 30 | Initial | Clear, colorless solution | 99.9 | <0.05 | 0.03 | 0.1 | 3.9 |
| | 40° C./1 M | Clear, colorless solution | 99.9 | <0.05 | 0.03 | 0.1 | — |
| | 40° C./2 M | Clear, colorless solution | 99.9 | <0.05 | 0.06 | 0.1 | — |
| | 40° C./3 M | Clear, colorless solution | 99.9 | <0.05 | 0.04 | 0.1 | — |
| | 40° C./6 M | Clear, colorless solution | 99.9 | <0.05 | 0.04 | 0.1 | 4.0 |
| | RT/1 M | Clear, colorless solution | 99.9 | <0.05 | 0.03 | 0.1 | — |
| | RT/3 M | Clear, colorless solution | 99.9 | <0.05 | 0.03 | 0.1 | — |
| | RT/6 M | Clear, colorless solution | 99.9 | <0.05 | 0.04 | 0.1 | — |
| | RT/9 M | Clear, colorless solution | 99.9 | <0.05 | 0.04 | 0.1 | 4.0 |
| 31 | Initial | Clear, colorless solution | 99.9 | <0.05 | 0.03 | 0.1 | 3.5 |
| | 40° C./1 M | Clear, colorless solution | 99.9 | <0.05 | 0.03 | 0.1 | — |
| | 40° C./2 M | Clear, colorless solution | 99.8 | <0.05 | 0.07 | 0.2 | — |
| | 40° C./3 M | Clear, colorless solution | 99.9 | <0.05 | 0.04 | 0.1 | — |
| | 40° C./6 M | Clear, colorless solution | 99.9 | <0.05 | 0.04 | 0.1 | 3.6 |
| | RT/1 M | Clear, colorless solution | 100.0 | <0.05 | 0.00 | 0.0 | — |
| | RT/3 M | Clear, colorless solution | 99.9 | <0.05 | 0.04 | 0.1 | — |
| | RT/6 M | Clear, colorless solution | 99.9 | <0.05 | 0.03 | 0.1 | — |
| | RT/9 M | Clear, colorless solution | 99.9 | <0.05 | 0.04 | 0.1 | 3.5 |
| 32 | Initial | Clear, colorless solution | 99.9 | <0.05 | 0.04 | 0.1 | 2.6 |
| | 40° C./1 M | Clear, colorless solution | 99.9 | <0.05 | 0.04 | 0.1 | — |
| | 40° C./2 M | Clear, colorless solution | 99.9 | <0.05 | 0.06 | 0.2 | — |
| | 40° C./3 M | Clear, colorless solution | 99.8 | <0.05 | 0.04 | 0.2 | — |
| | 40° C./6 M | Clear, colorless solution | 99.8 | <0.05 | 0.04 | 0.2 | 2.6 |
| | RT/1 M | Clear, colorless solution | 99.9 | <0.05 | 0.03 | 0.1 | — |
| | RT/3 M | Clear, colorless solution | 99.9 | <0.05 | 0.04 | 0.1 | — |
| | RT/6 M | Clear, colorless solution | 99.9 | <0.05 | 0.04 | 0.1 | — |
| | RT/9 M | Clear, colorless solution | 99.9 | <0.05 | 0.04 | 0.1 | 2.6 |
| 33 | Initial | Clear, colorless solution | 99.9 | 0.06 | 0.06 | 0.1 | 5.1 |
| | 40° C./1 M | Clear, colorless solution | 99.9 | <0.05 | 0.03 | 0.1 | — |
| | 40° C./2 M | Clear, colorless solution | 99.9 | <0.05 | 0.06 | 0.1 | — |
| | 40° C./3 M | Clear, colorless solution | 99.9 | <0.05 | 0.04 | 0.1 | — |
| | 40° C./6 M | Clear, colorless solution | 99.9 | <0.05 | 0.03 | 0.1 | 4.9 |
| | RT/1 M | Clear, colorless solution | 99.9 | <0.05 | 0.03 | 0.1 | — |
| | RT/3 M | Clear, colorless solution | 99.9 | <0.05 | 0.04 | 0.1 | — |
| | RT/6 M | Clear, colorless solution | 99.9 | <0.05 | 0.04 | 0.1 | — |
| | RT/9 M | Clear, colorless solution | 99.9 | <0.05 | 0.04 | 0.1 | 5.1 |

Examples 34-37 describe the liquid formulation of pitolisant hydrochloride, prepared using ingredients detailed in Table 18. Examples 34, 35 and 37 were prepared by dissolving all excipients one-by-one (where applicable-methylparaben, propylparaben, sucralose, strawberry flavor, tocopherol, polysorbate 80, water, and hydrochloric acid) in about 80% of required quantity of polyethylene glycol 400, followed by dissolving pitolisant hydrochloride. The volume of the solution was brought to the batch size using polyethylene glycol 400 and mixed to homogeneity. Example 36 was prepared by dissolving, pitolisant hydrochloride, citric acid and trisodium citrate, one-by-one, in required quantity of water and adding the prepared solution to about 80% of required quantity of polyethylene glycol 400. The volume of the batch was brought to batch size using polyethylene glycol 400 and was mixed to homogeneity. All prepared solutions were filled in containers, and sealed with closures with nitrogen overlay. Stability test results for example 34-37 are provided in Table 19.

TABLE 18
Compositions for pitolisant liquid formulation of example 34-37.

| | Example 34 | Example 35 | Example 36 | Example 37 |
|---|---|---|---|---|
| Ingredients | (% w/v) | (% w/v) | (% w/v) | (% w/v) |
| Pitolisant hydrochloride | 0.5% | 0.5% | 0.5% | 0.5% |
| Sucralose | — | 0.5% | — | 0.5% |
| Methylparaben | — | 0.25% | — | 0.25% |
| Propylparaben | — | 0.025% | — | 0.025% |
| Strawberry flavor | — | 0.1% | — | 0.1% |
| All-rac-Alpha-Tocopherol | — | 0.1% | — | 0.1% |
| Polysorbate 80 | — | 0.05% | — | 0.05% |
| Hydrochloric acid (0.1N) | — | — | — | 0.8% |
| Citric acid monohydrate | — | — | 0.2% | — |
| Tri-sodium citrate dihydrate | — | — | 0.05% | — |
| Purified Water | — | — | 20% | 20% |
| Polyethylene glycol 400 | q.s. to 100% | q.s. to 100% | q.s. to 100% | q.s. to 100% |

TABLE 19
Stability test results for example 34-37.

| | | | | | % Maximum | |
|---|---|---|---|---|---|---|
| Example | | | | % PTS | Single | % Total |
| No. | Time point | Visual Description | % Purity | N-Oxide | Impurity | Impurities |
| 34 | Initial | Clear, colorless solution | 100.0 | <0.05 | 0.00 | 0.0 |
| | 60° C./2 W | Clear, colorless solution | 99.8 | 0.15 | 0.15 | 0.2 |
| | 60° C./1 M | Clear, colorless solution | 99.7 | 0.26 | 0.26 | 0.3 |
| | 40° C./1 M | Clear, colorless solution | 99.8 | 0.16 | 0.16 | 0.2 |
| | 40° C./2 M | Clear, colorless solution | 99.7 | 0.23 | 0.23 | 0.3 |
| | 40° C./3 M | Clear, colorless solution | 99.7 | 0.27 | 0.27 | 0.3 |
| | 40° C./6 M | Clear, colorless solution | 99.6 | 0.30 | 0.30 | 0.4 |
| | RT/3 M | Clear, colorless solution | 99.2 | 0.73 | 0.73 | 0.8 |
| | RT/6 M | Clear, colorless solution | 99.2 | 0.58 | 0.58 | 0.8 |
| | RT/9 M | Clear, colorless solution | 99.3 | 0.53 | 0.53 | 0.7 |
| 35 | Initial | Clear, colorless solution | 99.9 | 0.08 | 0.08 | 0.1 |
| | 60° C./2 W | Clear, colorless solution | 99.8 | 0.07 | 0.07 | 0.2 |
| | 60° C./1 M | Clear, colorless solution | 99.8 | 0.09 | 0.09 | 0.2 |
| | 40° C./1 M | Clear, colorless solution | 99.9 | <0.05 | 0.03 | 0.1 |
| | 40° C./6 M | Clear, colorless solution | 99.7 | <0.05 | 0.11 | 0.3 |
| | RT/9 M | Clear, colorless solution | 99.8 | <0.05 | 0.07 | 0.2 |
| 36 | Initial | Clear, colorless solution | 100.0 | <0.05 | 0.03 | 0.1 |
| | 60° C./2 W | Clear, colorless solution | 99.9 | 0.05 | 0.05 | 0.1 |
| | 60° C./1 M | Clear, colorless solution | 99.8 | 0.06 | 0.06 | 0.2 |
| | 40° C./1 M | Clear, colorless solution | 99.8 | 0.12 | 0.12 | 0.2 |
| | 40° C./2 M | Clear, colorless solution | 99.6 | 0.32 | 0.32 | 0.4 |
| | 40° C./3 M | Clear, colorless solution | 99.6 | 0.27 | 0.27 | 0.4 |
| | 40° C./6 M | Clear, colorless solution | 99.6 | 0.28 | 0.28 | 0.4 |
| | RT/3 M | Clear, colorless solution | 99.9 | <0.05 | 0.04 | 0.1 |
| | RT/6 M | Clear, colorless solution | 99.8 | 0.07 | 0.07 | 0.2 |
| | RT/9 M | Clear, colorless solution | 99.4 | 0.51 | 0.51 | 0.6 |
| 37 | Initial | Clear, colorless solution | 99.9 | 0.08 | 0.08 | 0.1 |
| | 60° C./2 W | Clear, colorless solution | 99.8 | <0.05 | 0.08 | 0.2 |
| | 60° C./1 M | Clear, colorless solution | 99.8 | 0.07 | 0.07 | 0.2 |
| | 40° C./1 M | Clear, colorless solution | 99.9 | <0.05 | 0.04 | 0.2 |
| | 40° C./6 M | Clear, colorless solution | 99.7 | <0.05 | 0.08 | 0.3 |
| | RT/9 M | Clear, colorless solution | 99.8 | <0.05 | 0.04 | 0.2 |

Examples 38-49 describe the liquid formulation of pitolisant hydrochloride, prepared using ingredients detailed in Table 20. The preservatives (methylparaben and propylparaben) were added one-by-one to approximately 80% of the total required quantity of purified water, which was optionally heated to 50-60° C. to aid dissolution of preservatives. After complete dissolution, this solution was cooled to room temperature (20-25° C.). Subsequently, other excipients as required for the specific example—sucralose, strawberry flavor, citric acid monohydrate, tri-sodium citrate, di-sodium phosphate dihydrate, and sodium di-hydrogen phosphate monohydrate-were added and mixed until dissolved into the solution sequentially. The active ingredient, pitolisant hydrochloride, was added and mixed to homogeneity. For example 46-49, the pH was adjusted to about 6.6 using hydrochloric acid. Finally, the volume of the solution was brought to the target batch size with purified water and mixed to obtain clear colorless solution. The final formulations were optionally filtered to remove foreign particulate matter, filled into container-closure systems as described in Table 21, and sealed with a closure. Stability test results for example 38-49 are provided in Table 22.

TABLE 20
Compositions for pitolisant liquid formulation of example 38-49.

| | Example | Example | Example |
|---|---|---|---|
| | 38 to 41 | 42 to 45 | 46 to 49 |
| Ingredients | (% w/v) | (% w/v) | (% w/v) |
| Pitolisant hydrochloride | 0.5% | 0.5% | 0.5% |
| Sucralose | 0.25% | 0.25% | 0.25% |
| Methylparaben | 0.15% | 0.15% | 0.15% |
| Propylparaben | 0.015% | 0.015% | 0.015% |
| Strawberry flavor | 0.05% | 0.05% | 0.05% |
| Citric acid monohydrate | — | 0.1% | — |
| Tri-sodium citrate dihydrate | — | 0.025% | — |
| Di-sodium phosphate, dihydrate | — | — | 0.1% |
| Sodium di-hydrogen phosphate, | — | — | 0.02% |
| monohydrate | | | |
| Hydrochloric acid (0.1N) | — | — | q.s. to |
| | | | pH 6.6 |
| Purified Water | q.s. | q.s. | q.s. |
| | to 100% | to 100% | to 100% |

TABLE 21
Packaging for pitolisant liquid formulation of example 38-49.

| | | | Nitrogen | Orientation of |
|---|---|---|---|---|
| Example | | Fill | overlay | Container during |
| No. | Primary Packaging | Volume | (Yes/No) | Stability |
| 38 | 10 CC HDPE bottle | 8 mL | No | Horizontal |
| | with screw cap | | | |
| 39 | 10 CC HDPE bottle | 8 mL | No | Upright |
| | with screw cap | | | |
| 40 | 10 CC HDPE bottle | 8 mL | Yes | Upright |
| | with screw cap | | | |
| 41 | 10 mL glass vial | 8 mL | No | Upright |
| | with stopper and | | | |
| | seal | | | |
| 42 | 10 CC HDPE bottle | 8 mL | No | Horizontal |
| | with screw cap | | | |
| 43 | 10 CC HDPE bottle | 8 mL | No | Upright |
| | with screw cap | | | |
| 44 | 10 CC HDPE bottle | 8 mL | Yes | Upright |
| | with screw cap | | | |
| 45 | 10 mL glass vial | 8 mL | No | Upright |
| | with stopper and | | | |
| | seal | | | |
| 46 | 10 CC HDPE bottle | 8 mL | No | Horizontal |
| | with screw cap | | | |
| 47 | 10 CC HDPE bottle | 8 mL | No | Upright |
| | with screw cap | | | |
| 48 | 10 CC HDPE bottle | 8 mL | Yes | Upright |
| | with screw cap | | | |
| 49 | 10 mL glass vial | 8 mL | No | Upright |
| | with stopper and | | | |
| | seal | | | |

TABLE 22
Stability test results for example 38-49.

| | | | | | % | | |
|---|---|---|---|---|---|---|---|
| | | | | | Maximum | | |
| Example | Time | | % | % PTS | Single | % Total | |
| No. | point | Visual Description | Purity | N-Oxide | Impurity | Impurities | pH |
| 38 | Initial | Clear, colorless solution | 99.9 | <0.05 | 0.03 | 0.1 | 5.3 |
| | 40° C./3 M | Clear, colorless solution | 99.9 | <0.05 | 0.04 | 0.1 | — |
| | 40° C./6 M | Clear, colorless solution | 99.9 | <0.05 | 0.04 | 0.1 | — |
| | RT/6 M | Clear, colorless solution | 99.9 | <0.05 | 0.04 | 0.1 | — |
| | RT/9 M | Clear, colorless solution | 99.9 | <0.05 | 0.04 | 0.1 | 4.5 |
| 39 | Initial | Clear, colorless solution | 99.9 | <0.05 | 0.03 | 0.1 | 5.3 |
| | 40° C./1 M | Clear, colorless solution | 99.9 | <0.05 | 0.04 | 0.1 | — |
| | 40° C./2 M | Clear, colorless solution | 99.9 | <0.05 | 0.04 | 0.1 | — |
| | 40° C./3 M | Clear, colorless solution | 99.9 | <0.05 | 0.03 | 0.1 | — |
| | 40° C./6 M | Clear, colorless solution | 99.8 | <0.05 | 0.17 | 0.2 | 4.5 |
| | RT/3 M | Clear, colorless solution | 99.9 | <0.05 | 0.04 | 0.1 | — |
| | RT/6 M | Clear, colorless solution | 99.9 | <0.05 | 0.04 | 0.1 | — |
| 40 | Initial | Clear, colorless solution | 100.0 | <0.05 | 0.03 | 0.0 | 5.3 |
| | 40° C./1 M | Clear, colorless solution | 99.9 | <0.05 | 0.10 | 0.1 | — |
| | 40° C./2 M | Clear, colorless solution | 99.9 | <0.05 | 0.04 | 0.1 | — |
| | 40° C./3 M | Clear, colorless solution | 99.9 | <0.05 | 0.04 | 0.1 | — |
| | 40° C./6 M | Clear, colorless solution | 99.8 | <0.05 | 0.12 | 0.2 | — |
| | RT/3 M | Clear, colorless solution | 99.9 | <0.05 | 0.04 | 0.1 | — |
| | RT/6 M | Clear, colorless solution | 99.9 | <0.05 | 0.04 | 0.1 | — |
| 41 | Initial | Clear, colorless solution | 99.9 | <0.05 | 0.03 | 0.1 | 4.5 |
| | 40° C./1 M | Clear, colorless solution | 100.0 | <0.05 | 0.00 | 0.0 | — |
| | 40° C./2 M | Clear, colorless solution | 99.9 | <0.05 | 0.04 | 0.1 | — |
| | 40° C./3 M | Clear, colorless solution | 99.9 | <0.05 | 0.04 | 0.1 | — |
| | 40° C./6 M | Clear, colorless solution | 99.8 | <0.05 | 0.11 | 0.2 | 4.4 |
| | RT/3 M | Clear, colorless solution | 99.9 | <0.05 | 0.04 | 0.1 | — |
| | RT/6 M | Clear, colorless solution | 99.9 | <0.05 | 0.04 | 0.1 | — |
| | RT/9 M | Clear, colorless solution | 99.9 | <0.05 | 0.04 | 0.1 | 4.6 |
| 42 | Initial | Clear, colorless solution | 99.9 | <0.05 | 0.03 | 0.1 | 3.2 |
| | 40° C./3 M | Clear, colorless solution | 99.9 | <0.05 | 0.03 | 0.1 | — |
| | 40° C./6 M | Clear, colorless solution | 99.9 | <0.05 | 0.06 | 0.1 | — |
| | RT/6 M | Clear, colorless solution | 99.9 | <0.05 | 0.04 | 0.1 | — |
| | RT/9 M | Clear, colorless solution | 99.9 | <0.05 | 0.04 | 0.1 | 3.1 |
| 43 | Initial | Clear, colorless solution | 99.9 | <0.05 | 0.03 | 0.1 | 3.1 |
| | 40° C./1 M | Clear, colorless solution | 99.9 | <0.05 | 0.10 | 0.1 | — |
| | 40° C./2 M | Clear, colorless solution | 99.9 | <0.05 | 0.04 | 0.1 | — |
| | 40° C./3 M | Clear, colorless solution | 99.9 | <0.05 | 0.04 | 0.1 | — |
| | 40° C./6 M | Clear, colorless solution | 99.9 | <0.05 | 0.08 | 0.2 | 3.1 |
| | RT/3 M | Clear, colorless solution | 99.9 | <0.05 | 0.04 | 0.1 | — |
| | RT/6 M | Clear, colorless solution | 99.9 | <0.05 | 0.04 | 0.1 | — |
| 44 | Initial | Clear, colorless solution | 99.9 | <0.05 | 0.03 | 0.1 | 3.2 |
| | 40° C./1 M | Clear, colorless solution | 99.9 | <0.05 | 0.10 | 0.2 | — |
| | 40° C./2 M | Clear, colorless solution | 99.9 | <0.05 | 0.03 | 0.1 | — |
| | 40° C./3 M | Clear, colorless solution | 99.9 | <0.05 | 0.03 | 0.1 | — |
| | 40° C./6 M | Clear, colorless solution | 99.9 | <0.05 | 0.06 | 0.1 | — |
| | RT/3 M | Clear, colorless solution | 99.9 | <0.05 | 0.04 | 0.1 | — |
| | RT/6 M | Clear, colorless solution | 99.9 | <0.05 | 0.04 | 0.1 | — |
| 45 | Initial | Clear, colorless solution | 99.9 | <0.05 | 0.03 | 0.1 | 3.2 |
| | 40° C./1 M | Clear, colorless solution | 99.9 | <0.05 | 0.09 | 0.1 | — |
| | 40° C./2 M | Clear, colorless solution | 99.9 | <0.05 | 0.04 | 0.1 | — |
| | 40° C./3 M | Clear, colorless solution | 99.9 | <0.05 | 0.04 | 0.1 | — |
| | 40° C./6 M | Clear, colorless solution | 99.9 | <0.05 | 0.05 | 0.2 | 3.1 |
| | RT/3 M | Clear, colorless solution | 99.9 | <0.05 | 0.04 | 0.1 | — |
| | RT/6 M | Clear, colorless solution | 99.9 | <0.05 | 0.04 | 0.1 | — |
| | RT/9 M | Clear, colorless solution | 99.9 | <0.05 | 0.04 | 0.1 | 3.1 |
| 46 | Initial | Clear, colorless solution | 99.9 | <0.05 | 0.03 | 0.1 | — |
| | 40° C./3 M | Clear, colorless solution | 99.9 | <0.05 | 0.04 | 0.1 | — |
| | 40° C./6 M | Clear, colorless solution | 99.9 | <0.05 | 0.04 | 0.1 | — |
| | RT/6 M | Clear, colorless solution | 99.9 | <0.05 | 0.04 | 0.1 | — |
| | RT/9 M | Clear, colorless solution | 99.9 | <0.05 | 0.08 | 0.1 | 6.4 |
| 47 | Initial | Clear, colorless solution | 99.9 | <0.05 | 0.03 | 0.1 | 6.6 |
| | 40° C./1 M | Clear, colorless solution | 99.9 | <0.05 | 0.03 | 0.1 | — |
| | 40° C./2 M | Clear, colorless solution | 99.9 | <0.05 | 0.04 | 0.1 | — |
| | 40° C./3 M | Clear, colorless solution | 99.9 | <0.05 | 0.04 | 0.1 | — |
| | 40° C./6 M | Clear, colorless solution | 99.8 | <0.05 | 0.04 | 0.2 | 6.3 |
| | RT/3 M | Clear, colorless solution | 99.9 | <0.05 | 0.04 | 0.1 | — |
| | RT/6 M | Clear, colorless solution | 99.9 | <0.05 | 0.04 | 0.1 | — |
| 48 | Initial | Clear, colorless solution | 99.9 | <0.05 | 0.03 | 0.1 | 6.6 |
| | 40° C./1 M | Clear, colorless solution | 99.7 | <0.05 | 0.27 | 0.3 | — |
| | 40° C./2 M | Clear, colorless solution | 99.9 | 0.05 | 0.05 | 0.1 | — |
| | 40° C./3 M | Clear, colorless solution | 99.9 | <0.05 | 0.04 | 0.1 | — |
| | 40° C./6 M | Clear, colorless solution | 99.8 | <0.05 | 0.04 | 0.2 | — |
| | RT/3 M | Clear, colorless solution | 99.9 | <0.05 | 0.04 | 0.1 | — |
| | RT/6 M | Clear, colorless solution | 99.9 | <0.05 | 0.04 | 0.1 | — |
| 49 | Initial | Clear, colorless solution | 99.9 | <0.05 | 0.03 | 0.1 | 6.6 |
| | 40° C./1 M | Clear, colorless solution | 99.6 | <0.05 | 0.42 | 0.4 | — |
| | 40° C./2 M | Clear, colorless solution | 99.9 | <0.05 | 0.04 | 0.1 | — |
| | 40° C./3 M | Clear, colorless solution | 99.9 | <0.05 | 0.04 | 0.1 | — |
| | 40° C./6 M | Clear, colorless solution | 99.9 | <0.05 | 0.04 | 0.1 | 6.5 |
| | RT/3 M | Clear, colorless solution | 99.9 | <0.05 | 0.04 | 0.1 | — |
| | RT/6 M | Clear, colorless solution | 99.9 | <0.05 | 0.04 | 0.1 | — |
| | RT/9 M | Clear, colorless solution | 99.9 | <0.05 | 0.04 | 0.1 | 6.6 |

Examples 50-54 describe the liquid formulation of pitolisant hydrochloride, prepared using ingredients detailed in Table 23. Where applicable, the preservatives (methylparaben and propylparaben) were added one-by-one to approximately 80% of the total required quantity of purified water, which was optionally heated to 50-60° C. After complete dissolution of preservatives, the solution was cooled to room temperature (20-25° C.). Where applicable, sucralose, strawberry flavor, citric acid monohydrate, and tri-sodium citrate were added sequentially and mixed until dissolved. The active ingredient, pitolisant hydrochloride, was then added and mixed until it dissolved. Finally, the solution's volume was brought up to the batch size with purified water and mixed to homogeneity. The formulation was then optionally filtered, filled into containers, and sealed with closure. Packaging components for example 50-54 are provided in Table 24 and stability test results are provided in Table 25.

TABLE 23
Compositions for pitolisant liquid formulation of example 50-54.

| | Example | Example | Example |
|---|---|---|---|
| | 50-51 | 52-53 | 54 |
| Ingredients | (% w/v) | (% w/v) | (% w/v) |
| Pitolisant hydrochloride | 10% | 10% | 10% |
| Sucralose | — | 0.3% | 0.3% |
| Methylparaben | — | 0.2% | 0.2% |
| Propylparaben | — | 0.02% | 0.02% |
| Strawberry flavor | — | — | 0.1% |
| Citric acid monohydrate | — | — | 0.15% |
| Tri-sodium citrate dihydrate | — | — | 0.03% |
| Purified Water | q.s. to 100% | q.s. to 100% | q.s. to 100% |

TABLE 24
Packaging for pitolisant liquid formulation of example 50-54.

| Example | | Fill | Nitrogen overlay |
|---|---|---|---|
| No. | Primary Packaging | Volume | (Yes/No) |
| 50 | 2 mL amber glass vial with | ~1 mL | Yes |
| | stopper and seal | | |
| 51 | 2 mL amber glass vial with | ~1 mL | No |
| | stopper and seal | | |
| 52 | 2 mL amber glass vial with | ~1 mL | Yes |
| | stopper and seal | | |
| 53 | 2 mL amber glass vial with | ~1 mL | No |
| | stopper and seal | | |
| 54 | 2 mL amber glass vial with | ~1 mL | Yes |
| | stopper and seal | | |

TABLE 25
Stability test results for example 50-54.

| | | | | | % Maximum | |
|---|---|---|---|---|---|---|
| Example | | | | % PTS | Single | % Total |
| No. | Time point | Visual Description | % Purity | N-Oxide | Impurity | Impurities |
| 50 | Initial | Clear, colorless solution | 99.9 | <0.05 | 0.03 | 0.1 |
| | 40° C./1 M | Clear, colorless solution | 99.9 | <0.05 | 0.14 | 0.1 |
| | 40° C./2 M | Clear, colorless solution | 99.7 | <0.05 | 0.17 | 0.3 |
| | 40° C./3 M | Clear, colorless solution | 99.4 | 0.05 | 0.36 | 0.6 |
| | 40° C./6 M | Clear, colorless solution | 99.3 | 0.07 | 0.35 | 0.7 |
| | RT/3 M | Clear, colorless solution | 99.9 | <0.05 | 0.04 | 0.1 |
| | RT/6 M | Clear, colorless solution | 99.9 | <0.05 | 0.05 | 0.1 |
| 51 | Initial | Clear, colorless solution | 99.9 | <0.05 | 0.03 | 0.1 |
| | 40° C./1 M | Clear, colorless solution | 99.9 | 0.05 | 0.05 | 0.1 |
| | 40° C./2 M | Clear, colorless solution | 99.9 | <0.05 | 0.05 | 0.1 |
| | 40° C./3 M | Clear, colorless solution | 99.9 | <0.05 | 0.05 | 0.1 |
| | 40° C./6 M | Clear, colorless solution | 99.8 | <0.05 | 0.05 | 0.2 |
| 52 | Initial | Clear, colorless solution | 100.0 | <0.05 | 0.03 | 0.0 |
| | 40° C./1 M | Clear, colorless solution | 100.0 | <0.05 | 0.03 | 0.0 |
| | 40° C./2 M | Clear, colorless solution | 99.9 | <0.05 | 0.04 | 0.1 |
| | 40° C./3 M | Clear, colorless solution | 99.9 | <0.05 | 0.05 | 0.1 |
| | 40° C./6 M | Clear, colorless solution | 99.5 | 0.06 | 0.25 | 0.5 |
| | RT/3 M | Clear, colorless solution | 99.9 | <0.05 | 0.04 | 0.1 |
| | RT/6 M | Clear, colorless solution | 99.9 | <0.05 | 0.04 | 0.1 |
| 53 | Initial | Clear, colorless solution | 100.0 | <0.05 | 0.03 | 0.0 |
| | 40° C./1 M | Clear, colorless solution | 100.0 | <0.05 | 0.05 | 0.0 |
| | 40° C./2 M | Clear, colorless solution | 99.9 | <0.05 | 0.04 | 0.1 |
| | 40° C./3 M | Clear, colorless solution | 99.9 | <0.05 | 0.05 | 0.1 |
| | 40° C./6 M | Clear, colorless solution | 99.2 | 0.06 | 0.19 | 0.8 |
| 54 | Initial | Clear, colorless solution | 99.9 | <0.05 | 0.03 | 0.1 |
| | 40° C./1 M | Clear, colorless solution | 99.8 | <0.05 | 0.15 | 0.2 |
| | 40° C./2 M | Clear, colorless solution | 99.7 | <0.05 | 0.17 | 0.3 |
| | 40° C./3 M | Clear, colorless solution | 99.6 | <0.05 | 0.22 | 0.4 |
| | 40° C./6 M | Clear, colorless solution | 99.5 | 0.06 | 0.33 | 0.5 |
| | RT/3 M | Clear, colorless solution | 99.8 | <0.05 | 0.11 | 0.2 |
| | RT/6 M | Clear, colorless solution | 99.7 | <0.05 | 0.19 | 0.3 |

Examples 55-59 describe the liquid formulation of pitolisant hydrochloride, prepared using ingredients detailed in Table 26. Where applicable, the preservatives (methylparaben and propylparaben) were added one-by-one to approximately 80% of the total required quantity of purified water, which was optionally heated to 50-60° C. to aid dissolution of preservatives. After complete dissolution, this solution was cooled to room temperature (20-25° C.). Where applicable, sucralose, and strawberry flavor were added sequentially and mixed until dissolved. The active ingredient, pitolisant hydrochloride, was then added and mixed until it dissolved. Finally, the solution's volume was brought up to the batch size with purified water and mixed to homogeneity. The formulation was then optionally filtered, filled into small glass containers (also called microvials) with fill volume of about 125 μL and closed with rubber closures with minimal headspace. Stability test results for example 55-59 are provided in Table 27.

TABLE 26
Compositions for pitolisant liquid formulation of example 55-59.

| | Exam- | Exam- | Exam- | Exam- | Exam- |
|---|---|---|---|---|---|
| | ple | ple | ple | ple | ple |
| | 55 | 56 | 57 | 58 | 59 |
| Ingredients | (% w/v) | (% w/v) | (% w/v) | (% w/v) | (% w/v) |
| Pitolisant | 20% | 20% | 20% | 40% | 40% |
| hydrochloride | | | | | |
| Sucralose | — | — | 0.2% | — | 0.15% |
| Methylparaben | — | 0.16% | 0.16% | — | 0.12% |
| Propylparaben | — | 0.016% | 0.016% | — | 0.016% |
| Strawberry | — | — | 0.12% | — | 0.09% |
| flavor | | | | | |
| Purified Water | q.s. | q.s. | q.s. | q.s. | q.s. |
| | to 100% | to 100% | to 100% | to 100% | to 100% |

TABLE 27
Stability test results for example 55-59.

| | | | | | % Maximum | |
|---|---|---|---|---|---|---|
| Example | | | | % PTS | Single | % Total |
| No. | Time point | Visual Description | % Purity | N-Oxide | Impurity | Impurities |
| 55 | Initial | Clear, colorless solution | 99.9 | <0.05 | 0.04 | 0.1 |
| | 40° C./1 M | Clear, colorless solution | 99.9 | <0.05 | 0.04 | 0.1 |
| | 40° C./2 M | Clear, colorless solution | 99.8 | <0.05 | 0.06 | 0.2 |
| | 40° C./3 M | Clear, colorless solution | 99.8 | <0.05 | 0.07 | 0.2 |
| | 40° C./6 M | Clear, colorless solution | 99.9 | <0.05 | 0.05 | 0.1 |
| | RT/3 M | Clear, colorless solution | 99.9 | <0.05 | 0.04 | 0.1 |
| | RT/6 M | Clear, colorless solution | 99.9 | <0.05 | 0.04 | 0.1 |
| 56 | Initial | Clear, colorless solution | 99.9 | <0.05 | 0.04 | 0.1 |
| | 40° C./1 M | Clear, colorless solution | 99.9 | <0.05 | 0.04 | 0.1 |
| | 40° C./2 M | Clear, colorless solution | 99.9 | <0.05 | 0.04 | 0.1 |
| | 40° C./3 M | Clear, colorless solution | 99.9 | <0.05 | 0.03 | 0.1 |
| | 40° C./6 M | Clear, colorless solution | 99.9 | <0.05 | 0.04 | 0.1 |
| | RT/3 M | Clear, colorless solution | 99.9 | <0.05 | 0.04 | 0.1 |
| | RT/6 M | Clear, colorless solution | 99.9 | <0.05 | 0.04 | 0.1 |
| 57 | Initial | Clear, colorless solution | 99.9 | <0.05 | 0.04 | 0.1 |
| | 40° C./1 M | Clear, colorless solution | 99.9 | <0.05 | 0.05 | 0.1 |
| | 40° C./2 M | Clear, colorless solution | 99.9 | <0.05 | 0.07 | 0.1 |
| | 40° C./3 M | Clear, colorless solution | 99.9 | <0.05 | 0.04 | 0.1 |
| | 40° C./6 M | Clear, colorless solution | 99.9 | <0.05 | 0.06 | 0.1 |
| | RT/3 M | Clear, colorless solution | 99.9 | <0.05 | 0.04 | 0.1 |
| | RT/6 M | Clear, colorless solution | 99.9 | <0.05 | 0.04 | 0.1 |
| 58 | Initial | Clear, colorless solution | 99.9 | <0.05 | 0.04 | 0.1 |
| | 40° C./1 M | Clear, colorless solution | 99.5 | <0.05 | 0.28 | 0.5 |
| | 40° C./2 M | Clear, colorless solution | 99.0 | 0.07 | 0.57 | 1.0 |
| | 40° C./3 M | Clear, colorless solution | 99.2 | 0.08 | 0.47 | 0.8 |
| | 40° C./6 M | Clear, colorless solution | 98.9 | 0.14 | 0.67 | 1.1 |
| | RT/3 M | Clear, colorless solution | 99.9 | <0.05 | 0.06 | 0.1 |
| | RT/6 M | Clear, colorless solution | 99.8 | <0.05 | 0.07 | 0.2 |
| 59 | Initial | Clear, colorless solution | 99.9 | <0.05 | 0.04 | 0.1 |
| | 40° C./1 M | Clear, colorless solution | 99.5 | 0.04 | 0.30 | 0.5 |
| | 40° C./2 M | Clear, colorless solution | 99.1 | 0.06 | 0.53 | 0.9 |
| | 40° C./3 M | Clear, colorless solution | 99.3 | 0.07 | 0.40 | 0.7 |
| | 40° C./6 M | Clear, colorless solution | 98.9 | 0.14 | 0.66 | 1.1 |
| | RT/3 M | Clear, colorless solution | 99.8 | <0.05 | 0.07 | 0.2 |
| | RT/6 M | Clear, colorless solution | 99.6 | <0.05 | 0.16 | 0.4 |

Examples 60-68 describe the liquid formulation of pitolisant hydrochloride, prepared using ingredients detailed in Table 28. For example 60-62, sodium benzoate was added and dissolved in about 80% of total required quantity of purified water at room temperature and pH was adjusted to about 4.5 using dilute hydrochloric acid. For example 63-68, methylparaben and propylparaben were added and dissolved one-by-one in about 80% of total required quantity of purified water optionally heated at about 50-60° C. (for faster and complete dissolution of preservatives), and the resulting solution was cooled to room temperature (about 20-25° C.). Subsequently, where applicable, sucralose, strawberry flavor, or cherry flavor were added into the solutions sequentially and mixed until dissolved or homogenized. The active ingredient, pitolisant hydrochloride, was added and mixed to dissolve. Finally, the volume of the solution was brought to the target batch size with purified water and mixed to homogeneity. The final formulations were optionally filtered to remove particulate matter, filled into containers and sealed with closures. Packaging components for example 60-68 are provided in Table 29 and stability test results are provided in Table 30.

TABLE 28
Compositions for pitolisant liquid formulation of example 60-68.

| | Example | Example | Example |
|---|---|---|---|
| | 60-62 | 63-65 | 66-68 |
| Ingredients | (% w/v) | (% w/v) | (% w/v) |
| Pitolisant hydrochloride | 0.5% | 0.5% | 2% |
| Sucralose | 0.25% | 0.25% | 0.25% |
| Sodium benzoate | 0.1% | — | — |
| Methylparaben | — | 0.15% | 0.15% |
| Propylparaben | — | 0.015% | 0.015% |
| Strawberry flavor | 0.05% | — | — |
| Cherry flavor | — | 0.05% | 0.05% |
| Hydrochloric acid (0.1N) | q.s. to pH 4.5 | — | — |
| Purified Water | q.s. to 100% | q.s. to 100% | q.s. to 100% |

TABLE 29
Packaging and stability orientations for example 60-68.

| | | | | Orientation |
|---|---|---|---|---|
| | | Fill | Nitrogen | during |
| Exam- | | Vol- | overlay | stability |
| ple# | Primary Packaging | ume | (Yes/No) | testing |
| 60, 63, 66 | 10 CC HDPE bottle | 10 mL | No | Horizontal |
| | with screw cap | | | |
| 61, 64, 67 | 10 CC HDPE bottle | 10 mL | No | Upright |
| | with screw cap | | | |
| 62, 65, 68 | 10 CC HDPE bottle | 10 mL | Yes | Upright |
| | with screw cap | | | |

TABLE 30
Stability test results for example 60-68.

| | | | | | % | | |
|---|---|---|---|---|---|---|---|
| | | | | | Maximum | | |
| Example | | | % | % PTS | Single | % Total | |
| No. | Time point | Visual Description | Purity | N-Oxide | Impurity | Impurities | pH |
| 60 | Initial | Clear, colorless solution | 99.9 | <0.05 | 0.03 | 0.1 | 4.5 |
| | 40° C./3 M | Clear, colorless solution | 99.9 | <0.05 | 0.04 | 0.1 | — |
| | 40° C./6 M | Clear, colorless solution | 99.9 | <0.05 | 0.04 | 0.1 | 4.6 |
| | RT/3 M | Clear, colorless solution | 99.9 | <0.05 | 0.04 | 0.1 | — |
| | RT/6 M | Clear, colorless solution | 99.9 | <0.05 | 0.04 | 0.1 | 4.6 |
| 61 | Initial | Clear, colorless solution | 99.9 | <0.05 | 0.03 | 0.1 | 4.8 |
| | 40° C./1 M | Clear, colorless solution | 99.9 | <0.05 | 0.04 | 0.1 | — |
| | 40° C./2 M | Clear, colorless solution | 99.9 | <0.05 | 0.04 | 0.1 | — |
| | 40° C./3 M | Clear, colorless solution | 99.9 | <0.05 | 0.04 | 0.1 | — |
| | 40° C./6 M | Clear, colorless solution | 99.9 | <0.05 | 0.04 | 0.1 | 4.4 |
| | RT/3 M | Clear, colorless solution | 99.9 | <0.05 | 0.04 | 0.1 | — |
| | RT/6 M | Clear, colorless solution | 99.9 | <0.05 | 0.04 | 0.1 | 4.7 |
| 62 | Initial | Clear, colorless solution | 99.9 | <0.05 | 0.03 | 0.1 | 4.7 |
| | 40° C./3 M | Clear, colorless solution | 99.9 | <0.05 | 0.04 | 0.1 | — |
| 63 | Initial | Clear, colorless solution | 99.9 | <0.05 | 0.04 | 0.1 | 4.5 |
| | 40° C./3 M | Clear, colorless solution | 99.9 | <0.05 | 0.04 | 0.1 | — |
| | 40° C./6 M | Clear, colorless solution | 99.9 | <0.05 | 0.04 | 0.1 | 4.5 |
| | RT/3 M | Clear, colorless solution | 99.9 | <0.05 | 0.04 | 0.1 | — |
| | RT/6 M | Clear, colorless solution | 100.0 | <0.05 | 0.04 | 0.0 | 4.6 |
| 64 | Initial | Clear, colorless solution | 99.9 | <0.05 | 0.04 | 0.1 | 4.6 |
| | 40° C./1 M | Clear, colorless solution | 99.9 | <0.05 | 0.04 | 0.1 | — |
| | 40° C./2 M | Clear, colorless solution | 99.9 | <0.05 | 0.04 | 0.1 | — |
| | 40° C./3 M | Clear, colorless solution | 99.9 | <0.05 | 0.04 | 0.1 | — |
| | 40° C./6 M | Clear, colorless solution | 99.9 | <0.05 | 0.04 | 0.1 | 4.4 |
| | RT/3 M | Clear, colorless solution | 99.9 | <0.05 | 0.04 | 0.1 | — |
| | RT/6 M | Clear, colorless solution | 99.9 | <0.05 | 0.04 | 0.1 | 4.8 |
| 65 | Initial | Clear, colorless solution | 99.9 | <0.05 | 0.04 | 0.1 | 4.7 |
| | 40° C./3 M | Clear, colorless solution | 99.9 | <0.05 | 0.04 | 0.1 | — |
| 66 | Initial | Clear, colorless solution | 99.9 | <0.05 | 0.04 | 0.1 | 4.5 |
| | 40° C./3 M | Clear, colorless solution | 99.9 | <0.05 | 0.04 | 0.1 | — |
| | 40° C./6 M | Clear, colorless solution | 99.9 | <0.05 | 0.04 | 0.1 | 4.6 |
| | RT/3 M | Clear, colorless solution | 99.9 | <0.05 | 0.04 | 0.1 | — |
| | RT/6 M | Clear, colorless solution | 99.9 | <0.05 | 0.07 | 0.1 | 4.6 |
| 67 | Initial | Clear, colorless solution | 99.9 | <0.05 | 0.04 | 0.1 | 4.6 |
| | 40° C./1 M | Clear, colorless solution | 99.9 | <0.05 | 0.04 | 0.1 | — |
| | 40° C./2 M | Clear, colorless solution | 99.9 | <0.05 | 0.04 | 0.1 | — |
| | 40° C./3 M | Clear, colorless solution | 99.9 | <0.05 | 0.04 | 0.1 | — |
| | 40° C./6 M | Clear, colorless solution | 99.9 | <0.05 | 0.04 | 0.1 | 4.5 |
| | RT/6 M | Clear, colorless solution | 99.9 | <0.05 | 0.06 | 0.1 | 4.7 |
| 68 | Initial | Clear, colorless solution | 99.9 | <0.05 | 0.04 | 0.1 | 4.7 |
| | 40° C./6 M | Clear, colorless solution | 99.9 | <0.05 | 0.04 | 0.1 | 4.7 |

Examples 69-73 describe the liquid formulation of pitolisant hydrochloride, prepared using ingredients detailed in Table 31. Where applicable, sucralose and disodium edetate were dissolved in about 80% of total required quantity of purified water at room temperature (about 20-25° C.). Pitolisant hydrochloride was added and mixed until dissolved and homogenous solution was obtained. Where applicable, pH was adjusted to target using dilute sodium hydroxide. Required quantity of benzalkonium chloride was added and mixed to homogeneity. Required quantity of hypromellose (HPMC K4M) was added with continuous mixing and mixed until clear, homogenous solution was obtained. Finally, the volume of the solution was brought to the target batch size with purified water and mixed to homogeneity. The final formulations were optionally filtered to remove foreign particulate matter, filled into containers and closed with closure. Formulations of example 69 and 71 were filled in 10 CC HDPE container equipped with spray device and 10 mL fill without nitrogen overlay. Formulations for example 70, 72, and 73 were filled in glass container with screw cap without nitrogen overlay with minimal headspace. Stability test results, for example 69-73 are provided in Table 32.

TABLE 31
Compositions for pitolisant liquid formulation of example 69-73.

| | Exam- | Exam- | Exam- | Exam- | Exam- |
|---|---|---|---|---|---|
| | ple | ple | ple | ple | ple |
| | 69 | 70 | 71 | 72 | 73 |
| Ingredients | (% w/v) | (% w/v) | (% w/v) | (% w/v) | (% w/v) |
| Pitolisant | 10% | 10% | 10% | 10% | 10% |
| hydrochloride | | | | | |
| Sucralose | — | 0.25% | 0.5% | 1% | 1% |
| Benzalkonium chloride | — | 0.02% | 0.02% | 0.02% | 0.02% |
| Disodium edetate | — | — | 0.1% | 0.1% | 0.1% |
| dihydrate | | | | | |
| Sodium hydroxide (1N) | — | — | — | q.s. to | q.s. to |
| | | | | pH 5.5 | pH 5.5 |
| Hydroxypropyl | — | 0.05% | — | 0.06% | 0.5% |
| methylcellulose (HPMC - | | | | | |
| Benecel ® K4M) | | | | | |
| Purified Water | q.s. | q.s. | q.s. | q.s. | q.s. |
| | to 100% | to 100% | to 100% | to 100% | to 100% |

TABLE 32
Stability test results for example 69-73.

| | | | | | % Maximum | |
|---|---|---|---|---|---|---|
| Example | | | | % PTS | Single | % Total |
| No. | Time point | Visual Description | % Purity | N-Oxide | Impurity | Impurities |
| 69 | Initial | Clear, colorless solution | 99.9 | <0.05 | 0.04 | 0.1 |
| | 40° C./1 M | Clear, colorless solution | 99.2 | 0.08 | 0.32 | 0.8 |
| | 40° C./3 M | Clear, colorless solution | 98.3 | 0.11 | 0.50 | 1.7 |
| | RT/3 M | Clear, colorless solution | 99.5 | <0.05 | 0.21 | 0.5 |
| 70 | Initial | Clear, colorless solution | 99.9 | <0.05 | 0.05 | 0.1 |
| | 40° C./1 M | Clear, colorless solution | 99.7 | <0.05 | 0.12 | 0.3 |
| | 40° C./2 M | Clear, colorless solution | 98.9 | 0.07 | 0.29 | 1.1 |
| | 40° C./3 M | Clear, colorless solution | 98.6 | 0.09 | 0.37 | 1.4 |
| | RT/2 M | Clear, colorless solution | 99.8 | <0.05 | 0.10 | 0.2 |
| | RT/3 M | Clear, colorless solution | 99.6 | <0.05 | 0.14 | 0.4 |
| 71 | Initial | Clear, colorless solution | 99.9 | <0.05 | 0.04 | 0.1 |
| | 40° C./1 M | Clear, colorless solution | 99.9 | <0.05 | 0.05 | 0.1 |
| | 40° C./3 M | Clear, colorless solution | 99.7 | <0.05 | 0.09 | 0.3 |
| | RT/3 M | Clear, colorless solution | 99.9 | <0.05 | 0.04 | 0.1 |
| 72 | Initial | Clear, colorless solution | 99.9 | <0.05 | 0.04 | 0.1 |
| | 40° C./1 M | Clear, colorless solution | 99.9 | <0.05 | 0.04 | 0.1 |
| 73 | Initial | Clear, colorless solution | 99.9 | <0.05 | 0.04 | 0.1 |
| | 40° C./1 M | Clear, colorless solution | 99.9 | <0.05 | 0.04 | 0.1 |

Examples 74-81 describe the liquid formulation of pitolisant hydrochloride, prepared using ingredients detailed in Table 33. Where applicable, sucralose, sodium benzoate, and strawberry flavor were added and dissolved in about 80% of total required quantity of purified water at room temperature (about 20-25° C.). pH was adjusted to about 4.5 using dilute hydrochloric acid. Pitolisant hydrochloride was added and mixed until dissolved and homogenous solution was obtained. Where applicable, required quantity of polyvinylpyrrolidone (PVP) or povidone K-12, povidone K-29/32, and povidone K-90 were added in the prepared solution with continuous mixing and mixed until clear, homogenous solution was obtained. Finally, the volume of the solution was brought to the target batch size with purified water and mixed to homogeneity. The final formulations were filtered to remove particulate matter, filled into glass containers with minimal headspace and closed with closure. Stability test results, for example 74-81 are provided in Table 34.

TABLE 33
Compositions for pitolisant liquid formulation of example 74-81.

| | Example No. (Quantity in % w/v) | | | | | | | |
|---|---|---|---|---|---|---|---|---|
| Ingredients | 74 | 75 | 76 | 77 | 78 | 79 | 80 | 81 |
| Pitolisant hydrochloride | 0.4% | 4% | 0.4% | 0.4% | 0.4% | 4% | 4% | 4% |
| Sucralose | — | — | 0.25% | 0.25% | 0.25% | 0.25% | 0.25% | 0.25% |
| Sodium benzoate | — | — | 0.1% | 0.1% | 0.1% | 0.1% | 0.1% | 0.1% |
| Strawberry flavor | — | — | 0.1% | 0.1% | 0.1% | 0.1% | 0.1% | 0.1% |
| Povidone K-12 | — | — | 1% | — | — | 1% | — | — |
| Povidone K-29/32 | — | — | — | 7% | — | — | 7% | — |
| Povidone K-90 | — | — | — | — | 7% | — | — | 7% |
| Hydrochloric acid (1N) | — | — | q.s. to | q.s. to | q.s. to | q.s. to | q.s. to | q.s. to |
| | | | pH 4.5 | pH 4.5 | pH 4.5 | pH 4.5 | pH 4.5 | pH 4.5 |
| Purified Water | q.s. to | q.s. to | q.s. to | q.s. to | q.s. to | q.s. to | q.s. to | q.s. to |
| | 100% | 100% | 100% | 100% | 100% | 100% | 100% | 100% |

TABLE 34
Stability test results for example 74-81.

| | | | | | % Maximum | |
|---|---|---|---|---|---|---|
| Example | | | | % PTS | Single | % Total |
| No. | Time point | Visual Description | % Purity | N-Oxide | Impurity | Impurities |
| 74 | Initial | Clear, colorless solution | 99.9 | <0.05 | 0.04 | 0.1 |
| | 40° C./2 M | Clear, colorless solution | 99.9 | <0.05 | 0.04 | 0.1 |
| | 40° C./3 M | Clear, colorless solution | 99.9 | <0.05 | 0.04 | 0.1 |
| | RT/3 M | Clear, colorless solution | 99.9 | <0.05 | 0.04 | 0.1 |
| 75 | Initial | Clear, colorless solution | 99.9 | <0.05 | 0.04 | 0.1 |
| | 40° C./2 M | Clear, colorless solution | 99.9 | <0.05 | 0.04 | 0.1 |
| | 40° C./3 M | Clear, colorless solution | 99.9 | <0.05 | 0.05 | 0.1 |
| | RT/3 M | Clear, colorless solution | 99.9 | <0.05 | 0.04 | 0.1 |
| 76 | Initial | Clear, colorless solution | 99.9 | <0.05 | 0.05 | 0.1 |
| | 40° C./2 M | Clear, colorless solution | 99.9 | <0.05 | 0.04 | 0.1 |
| | 40° C./3 M | Clear, colorless solution | 99.9 | <0.05 | 0.02 | 0.1 |
| | RT/3 M | Clear, colorless solution | 99.9 | <0.05 | 0.03 | 0.1 |
| 77 | Initial | Clear, colorless solution | 100.0 | <0.05 | 0.03 | 0.0 |
| | 40° C./2 M | Clear, colorless solution | 99.9 | <0.05 | 0.05 | 0.1 |
| | 40° C./3 M | Clear, very slight yellow | 99.7 | <0.05 | 0.21 | 0.3 |
| | | color solution | | | | |
| | RT/3 M | Clear, colorless solution | 99.8 | <0.05 | 0.11 | 0.2 |
| 78 | Initial | Clear, colorless solution | 99.9 | <0.05 | 0.03 | 0.1 |
| | 40° C./2 M | Clear, colorless solution | 99.8 | <0.05 | 0.06 | 0.2 |
| | 40° C./3 M | Clear, colorless solution | 99.7 | <0.05 | 0.08 | 0.3 |
| | RT/3 M | Clear, colorless solution | 99.9 | <0.05 | 0.04 | 0.1 |
| 79 | Initial | Clear, colorless solution | 99.9 | <0.05 | 0.04 | 0.1 |
| | 40° C./2 M | Clear, colorless solution | 99.7 | <0.05 | 0.08 | 0.3 |
| | 40° C./3 M | Clear, colorless solution | 99.7 | <0.05 | 0.11 | 0.3 |
| | RT/3 M | Clear, colorless solution | 99.8 | <0.05 | 0.07 | 0.2 |
| 80 | Initial | Clear, colorless solution | 99.9 | <0.05 | 0.04 | 0.1 |
| | 40° C./2 M | Clear, colorless solution | 99.8 | <0.05 | 0.12 | 0.2 |
| | 40° C./3 M | Clear, very slight yellow | 99.7 | <0.05 | 0.15 | 0.3 |
| | | color solution | | | | |
| | RT/3 M | Clear, colorless solution | 99.8 | <0.05 | 0.07 | 0.2 |
| 81 | Initial | Clear, colorless solution | 99.9 | <0.05 | 0.04 | 0.1 |
| | 40° C./2 M | Clear, colorless solution | 99.7 | 0.06 | 0.09 | 0.3 |
| | 40° C./3 M | Clear, colorless solution | 99.5 | 0.09 | 0.18 | 0.5 |
| | RT/3 M | Clear, colorless solution | 99.8 | <0.05 | 0.06 | 0.2 |

Examples 82-88 describe the liquid formulation of pitolisant hydrochloride, prepared using ingredients detailed in Table 35. Where applicable, sucralose, sodium benzoate, and cherry flavor were added and mixed to dissolved in about 80% of total required quantity of purified water or Neosorb® 70/70 (70% sorbitol solution) at room temperature (about 20-25° C.). Where applicable, pH was adjusted to about 4.5 using dilute hydrochloric acid. Pitolisant hydrochloride was added and mixed until dissolved and homogenous solution was obtained. Where applicable, required quantity of hypromellose (HPMC K4M) was added in the prepared solution with continuous mixing and mixed until clear, homogenous solution was obtained. Finally, the volume of the solution was brought to the target batch size with purified water or Neosorb® solution and mixed to homogeneity. The final formulations were optionally filtered to remove particulate matter, filled into containers and closed with closure. Stability test results, for example 82-88 are provided in Table 36.

TABLE 35
Compositions for pitolisant liquid formulation of example 82-88.

| | Example No. (Quantity in % w/v) | | | | | | |
|---|---|---|---|---|---|---|---|
| Ingredients | 82 | 83 | 84 | 85 | 86 | 87 | 88 |
| Pitolisant hydrochloride | 0.4% | 0.4% | 4% | 4% | 0.4% | 4% | 2% |
| Sucralose | 0.25% | 0.25% | 0.25% | 0.25% | 1% | 1% | — |
| Sodium benzoate | 0.1% | 0.1% | 0.1% | 0.1% | 0.1% | 0.1% | — |
| Cherry flavor | 0.1% | 0.1% | 0.1% | 0.1% | 0.5% | 0.5% | — |
| Hydrochloric acid (1N) | q.s. to | q.s. to | q.s. to | q.s. to | — | — | — |
| | pH 4.5 | pH 4.5 | pH 4.5 | pH 4.5 | | | |
| Hydroxypropyl methylcellulose | 0.05% | 0.5% | 0.05% | 0.5% | 0.06% | 1% | — |
| (HPMC - Benecel ® K4M) | | | | | | | |
| Neosorb ® 70/70 (Sorbitol | — | — | — | — | — | — | q.s. to |
| solution - 70% w/v) | | | | | | | 100% |
| Purified Water | q.s. to | q.s. to | q.s. to | q.s. to | q.s. to | q.s. to | — |
| | 100% | 100% | 100% | 100% | 100% | 100% | |

TABLE 36
Stability test results for example 82-88.

| | | | | | % Maximum | |
|---|---|---|---|---|---|---|
| Example | | | | % PTS | Single | % Total |
| No. | Time point | Visual Description | % Purity | N-Oxide | Impurity | Impurities |
| 82 | Initial | Clear, colorless solution | 99.9 | <0.05 | 0.04 | 0.1 |
| | 40° C./1 M | Clear, colorless solution | 99.9 | <0.05 | 0.04 | 0.1 |
| | 40° C./2 M | Clear, colorless solution | 99.9 | <0.05 | 0.04 | 0.1 |
| | 40° C./3 M | Clear, colorless solution | 99.9 | <0.05 | 0.04 | 0.1 |
| | RT/3 M | Clear, colorless solution | 99.9 | <0.05 | 0.04 | 0.1 |
| 83 | Initial | Clear, colorless solution | 99.9 | <0.05 | 0.04 | 0.1 |
| | 40° C./1 M | Clear, colorless solution | 99.9 | <0.05 | 0.04 | 0.1 |
| | 40° C./2 M | Clear, colorless solution | 99.9 | <0.05 | 0.04 | 0.1 |
| | 40° C./3 M | Clear, colorless solution | 99.9 | <0.05 | 0.04 | 0.1 |
| | RT/3 M | Clear, colorless solution | 99.9 | <0.05 | 0.04 | 0.1 |
| 84 | Initial | Clear, colorless solution | 99.9 | <0.05 | 0.04 | 0.1 |
| | 40° C./1 M | Clear, colorless solution | 99.8 | <0.05 | 0.06 | 0.2 |
| | 40° C./2 M | Clear, colorless solution | 99.7 | <0.05 | 0.10 | 0.3 |
| | 40° C./3 M | Clear, colorless solution | 99.7 | <0.05 | 0.12 | 0.3 |
| | RT/3 M | Clear, colorless solution | 99.9 | <0.05 | 0.05 | 0.1 |
| 85 | Initial | Clear, colorless solution | 99.9 | <0.05 | 0.04 | 0.1 |
| | 40° C./1 M | Clear, colorless solution | 99.8 | <0.05 | 0.04 | 0.2 |
| | 40° C./2 M | Clear, colorless solution | 99.8 | <0.05 | 0.06 | 0.2 |
| | 40° C./3 M | Clear, colorless solution | 99.8 | <0.05 | 0.08 | 0.2 |
| | RT/3 M | Clear, colorless solution | 99.9 | <0.05 | 0.04 | 0.1 |
| 86 | Initial | Clear, colorless solution | 99.8 | <0.05 | 0.04 | 0.2 |
| | 40° C./1 M | Clear, colorless solution | 99.9 | <0.05 | 0.04 | 0.1 |
| 87 | Initial | Clear, colorless solution | 99.9 | <0.05 | 0.04 | 0.1 |
| | 40° C./1 M | Clear, colorless solution | 99.9 | <0.05 | 0.04 | 0.1 |
| 88 | Initial | Clear, colorless solution | 99.9 | <0.05 | 0.04 | 0.1 |
| | 40° C./1 M | Clear, colorless solution | 99.9 | <0.05 | 0.04 | 0.1 |

Examples 89-93 describe the liquid formulation of pitolisant hydrochloride, prepared using ingredients detailed in Table 37. Where applicable, sodium chloride, mannitol and dextrose were added and mixed to dissolved in about 80% of total required quantity of water for injection at room temperature (about 20-25° C.). Finally, the volume of the solution was brought to the target batch size with water for injection and mixed to homogeneity. The final formulations were sterile filtered using 0.22μ filter, filled into USP type I glass vials with 2 mL fill volume, closed with stopper and sealed with aluminum crimp seals. Stability test results, for example 89-93 are provided in Table 38.

TABLE 37
Compositions for pitolisant liquid formulation of example 89-93.

| | Example No. (Quantity in % w/v) | | | | |
|---|---|---|---|---|---|
| Ingredients | 89 | 90 | 91 | 92 | 93 |
| Pitolisant hydrochloride | 0.4% | 0.4% | 4% | 4% | 10% |
| Sodium chloride | — | 0.9% | — | — | — |
| Mannitol | — | — | 5% | — | — |
| Dextrose | — | — | — | 5% | — |
| Water for injection | q.s. | q.s. | q.s. | q.s. | q.s. |
| | to 100% | to 100% | to 100% | to 100% | to 100% |

TABLE 38
Stability test results for example 89-93.

| | | | | | % Maximum | |
|---|---|---|---|---|---|---|
| Example | | | | % PTS | Single | % Total |
| No. | Time point | Visual Description | % Purity | N-Oxide | Impurity | Impurities |
| 89 | 121° C./15 | Clear, colorless solution | 100.0 | <0.05 | 0.02 | 0.0 |
| | minutes | | | | | |
| 90 | 121° C./15 | Clear, colorless solution | 100.0 | <0.05 | 0.02 | 0.0 |
| | minutes | | | | | |
| 91 | 121° C./15 | Clear, colorless solution | 100.0 | <0.05 | 0.02 | 0.0 |
| | minutes | | | | | |
| 92 | 121° C./15 | Clear, colorless solution | 100.0 | <0.05 | 0.02 | 0.0 |
| | minutes | | | | | |
| 93 | 121° C./15 | Clear, colorless solution | 100.0 | <0.05 | 0.02 | 0.0 |
| | minutes | | | | | |

Example 94 describes the liquid formulation of pitolisant free base, prepared using ingredients detailed in Table 39. Pitolisant hydrochloride was dissolved in about 70% of batch required quantity of water and sodium hydroxide was slowly added to neutralize pitolisant hydrochloride salt into pitolisant free base and until cloudy dispersion was obtained. Volume of the batch was made up to the batch size using water. The dispersion was filled into 2R USP type I glass vial, stoppered and crimped with aluminum crimp seal. Stability test results provided in Table 40.

TABLE 39
Compositions for pitolisant liquid formulation of example 94.

| Ingredients | Example No. 94 (Quantity in % w/v) |
|---|---|
| Pitolisant hydrochloride | 0.1% |
| Sodium hydroxide (1N) | q.s. to pH 7.5 |
| Water | q.s. to 100% |

TABLE 40
Stability test results for example 94.

| | | | | | % Maximum | |
|---|---|---|---|---|---|---|
| Example | | | | % PTS | Single | % Total |
| No. | Time point | Visual Description | % Purity | N-Oxide | Impurity | Impurities |
| 94 | 121° C./15 | Cloudy dispersion | 99.9 | <0.05 | 0.03 | 0.1 |
| | minutes | | | | | |

These results indicate the feasibility of pitolisant free base dispersion that can be further optimized using suitable excipients for long term storage and intended use. pH can be further increased to fully neutralize the pitolisant salt based on desired product profile and intended site of administration.

Example 95-98 describes the liquid formulation of pitolisant hydrochloride and/or its free base, prepared using ingredients detailed in Table 41. Where applicable, the preservatives (methylparaben and propylparaben) were added one-by-one to approximately 80% of the total required quantity of purified water, which was optionally heated to 50-60° C. to aid dissolution of preservatives. The resulting preservative solution was then cooled to room temperature (about 20-25° C.). Subsequently, sucralose and cherry flavor were added into the solution sequentially and mixed until dissolved or homogenized. The active ingredient, pitolisant hydrochloride, was added and mixed to homogeneity. For example 96, xanthan gum was added with continuous mixing and mixed to homogeneity followed by pH was adjusted to 8.5 by slow addition of sodium hydroxide with rapid mixing. For example 97 and 98, hydroxypropyl-β-cellulose was added and mixed until dissolved. pH was adjusted to 8.5 by slow addition of sodium hydroxide. For example 98, xanthan gum was added with continuous mixing and mixed to homogeneity. Finally, the volume of the formulations were brought to the target batch size with purified water and mixed to homogeneity. The final formulations were filled into containers and closed with closures. Stability test results provided in Table 42.

TABLE 41
Compositions for pitolisant liquid formulation of example 95-98.

| | Example No. (Quantity in % w/v) | | | |
|---|---|---|---|---|
| Ingredients | 95 | 96 | 97 | 98 |
| Pitolisant | 1% | 1% | 1% | 1% |
| hydrochloride | | | | |
| Sucralose | 0.5% | 0.5% | 0.5% | 0.5% |
| Methylparaben | 0.125% | 0.125% | 0.125% | 0.125% |
| Propylparaben | 0.0125% | 0.0125% | 0.0125% | 0.0125% |
| Cherry flavor | 0.1% | 0.1% | 0.1% | 0.1% |
| Sodium | — | q.s. to | q.s. to | q.s. to |
| hydroxide (1N) | | pH 8.5 | pH 8.5 | pH 8.5 |
| Xanthan gum | — | 0.3% | — | 0.3% |
| Hydroxy- | — | — | 7.5% | 7.5% |
| propyl-β- | | | | |
| cyclodextrin | | | | |
| Purified water | q.s. | q.s. | q.s. | q.s. |
| | to 100% | to 100% | to 100% | to 100% |

TABLE 42
Stability test results for example 95-98.

| | | | | | % Maximum | |
|---|---|---|---|---|---|---|
| Example | | | | % PTS | Single | % Total |
| No. | Time point | Visual Description | % Purity | N-Oxide | Impurity | Impurities |
| 95 | Initial | Clear, colorless solution | 99.9 | <0.05 | 0.04 | 0.1 |
| | 40° C./10 days | Clear, colorless solution | 99.9 | <0.05 | 0.04 | 0.1 |
| 96 | Initial | Clear, colorless solution | 100.0 | <0.05 | 0.03 | 0.0 |
| | 40° C./10 days | Clear, colorless solution | 99.9 | <0.05 | 0.04 | 0.1 |
| 97 | Initial | Off-white color dispersion | 100.0 | <0.05 | 0.04 | 0.0 |
| | 40° C./10 days | Off-white color dispersion | 99.9 | <0.05 | 0.04 | 0.1 |
| 98 | Initial | Clear, colorless solution | 100.0 | <0.05 | 0.04 | 0.0 |
| | 40° C./10 days | Clear, colorless solution | 99.9 | <0.05 | 0.04 | 0.1 |

Example 99-102 describes the liquid formulation of pitolisant, prepared using ingredients detailed in Table 43. Where applicable, disodium edetate and benzalkonium chloride were added to about 80% of required quantity of water and mixed to dissolve. The active ingredient, pitolisant hydrochloride, was added and mixed to homogeneity. For example 102, pH was adjusted to 6.2 using sodium hydroxide. Finally, the volume of the formulations were brought to the target batch size with purified water and mixed to homogeneity. The final formulations were filled into 2R clear USP Type I glass vial with fill volume of 0.65 mL with (example 101, 102) or without nitrogen overlay (example 99, 100), stoppered, and crimp sealed. The samples were autoclaved at 121° C. for 15 minutes. Stability test results provided in Table 44.

TABLE 43
Compositions for pitolisant liquid formulation of example 99-102.

| | Example No. (Quantity in % w/v) | | | |
|---|---|---|---|---|
| Ingredients | 99 | 100 | 101 | 102 |
| Pitolisant hydrochloride | 40% | 40% | 40% | 40% |
| Benzalkonium chloride | — | 0.1% | 0.1% | — |
| Disodium edetate dihydrate | — | 0.05% | 0.05% | — |
| Sodium hydroxide (1N) | — | — | — | q.s. to |
| | | | | pH 6.2 |
| Purified water | q.s. | q.s. | q.s. | q.s. |
| | to 100% | to 100% | to 100% | to 100% |
| Nitrogen overlay | No | No | Yes | Yes |

TABLE 44
Stability test results for example 99-102.

| | | | | | % Maximum | |
|---|---|---|---|---|---|---|
| Example | | | | % PTS | Single | % Total |
| No. | Time point | Visual Description | % Purity | N-Oxide | Impurity | Impurities |
| 99 | 121° C./15 | Clear, colorless | 99.7 | <0.05 | 0.07 | 0.3 |
| | minutes | solution | | | | |
| 100 | 121° C./15 | Clear, colorless | 99.9 | <0.05 | 0.03 | 0.1 |
| | minutes | solution | | | | |
| 101 | 121° C./15 | Clear, colorless | 100.0 | <0.05 | 0.02 | 0.0 |
| | minutes | solution | | | | |
| 102 | 121° C./15 | Clear, colorless | 100.0 | <0.05 | 0.02 | 0.0 |
| | minutes | solution | | | | |

Example 103-110 were prepared by adding organic alkalizer in 400 mg/mL pitolisant hydrochloride solution in aqueous and non-aqueous medium. Samples were prepared as given in table 45, by transferring about 0.65 mL of pitolisant hydrochloride solution (400 mg/mL) in 2R USP Type I glass vial, an alkalizer was added in specified quantity and mixed on vortex. The vials were stoppered, crimp sealed with aluminum seal, autoclaved at about 121° C. for 15 minutes and visual appearance recorded after treatment and tested using HPLC.

TABLE 45
Compositions and test results for example 103-110.

| | | | Nitro- | |
|---|---|---|---|---|
| Exam- | | | gen | Visual |
| ple | | | Overlay | appearance |
| No. | Vehicle | Alkalizer/quantity | (Yes/No) | after treatment |
| 103 | Propylene | Tromethamine (25 mg) | Yes | Colorless liquid |
| | Glycol | | | |
| 104 | Glycerin | Tromethamine (25 mg) | Yes | Colorless liquid |
| 105 | Propylene | Tromethamine (25 mg) | No | Yellow color |
| | Glycol | | | liquid |
| 106 | Glycerin | L-Arginine (25 mg) | No | Orange color |
| | | | | liquid |
| 107 | Propylene | L-Arginine (25 mg) | Yes | Colorless liquid |
| | glycol | | | |
| 108 | Purified | L-Arginine (25 mg) | Yes | Colorless liquid |
| | water | | | |
| 109 | Purified | Tromethamine (25 mg) | Yes | Colorless liquid |
| | water | | | |
| 110 | Glycerin | Tromethamine (25 mg) | No | Orange color |
| | | | | liquid |

TABLE 46
HPLC test results for example 103-109 after
autoclave at 121° C. for 15 minutes.

| Example | % | % Maximum | % Total |
|---|---|---|---|
| No. | Purity | Single Impurity | Impurities |
| 103 | 99.9 | 0.02 | 0.1 |
| 104 | 99.9 | 0.02 | 0.1 |
| 105 | 99.9 | 0.03 | 0.1 |
| 106 | 99.9 | 0.09 | 0.1 |
| 107 | 99.9 | 0.02 | 0.1 |
| 108 | 99.9 | 0.02 | 0.1 |
| 109 | 99.9 | 0.02 | 0.1 |

### Prophetic Examples

Following are the additional examples which were not prepared by the inventor but could be prepared based on the present disclosure.

Examples 111-116 describe the liquid formulation of pitolisant hydrochloride, that could be prepared using ingredients detailed in Table 47. The formulations could be prepared by dissolving sucralose, disodium edetate, benzalkonium chloride, n-dodecyl beta-D-maltoside, one-by-one, in about 80% of the required quantity of purified water. pH is adjusted between 4 and 6 using sodium hydroxide and/or hydrochloric acid. HPMC is added and mixed until dissolved and homogenized. Pitolisant hydrochloride is added and mixed until dissolved. Volume is made up to the batch size using purified water. The solution is optionally filtered and filled into containers such as plastic bottles or glass bottles and closed with closures. These formulations are suitable for intranasal administration.

TABLE 47
Liquid formulation of pitolisant for intranasal use

| | Example No. - Quantity (% w/v) | | | | | |
|---|---|---|---|---|---|---|
| Ingredient | 111 | 112 | 113 | 114 | 115 | 116 |
| Pitolisant | 0.4-40% | 10-40% | 20% | 0.4-40% | 10-40% | 20% |
| hydrochloride | | | | | | |
| Sucralose | 0.5-5% | 1-5% | 2% | 0.5-5% | 1-5% | 2% |
| Benzalkonium chloride | 0.001-0.2% | 0.1-0.2% | 0.1% | 0.001-0.2% | 0.1-0.2% | 0.1% |
| Disodium edetate | 0.001-0.5% | 0.1-0.2% | 0.15% | 0.001-0.5% | 0.1-0.2% | 0.15% |
| Hypromellose (HPMC | 0.05-0.5% | 0.1-0.2% | 0.1% | 0.05-0.5% | 0.1-0.2% | 0.1% |
| K4M) | | | | | | |
| n-dodecyl beta-D- | 0.001-1% | 0.1-0.5% | 0.2% | 0.001-1% | 0.1-0.5% | 0.3% |
| maltoside | | | | | | |
| Sodium | q.s. to pH 4 | q.s. to pH 4 | q.s. to pH 4 | q.s. to pH 4 | q.s. to pH 4 | q.s. to pH 4 |
| hydroxide/hydrochloric | to 6 | to 6 | to 6 | to 6 | to 6 | to 6 |
| acid (optional) | | | | | | |
| Purified Water | q.s. to | q.s. to | q.s. to | q.s. to | q.s. to | q.s. to |
| | 100% | 100% | 100% | 100% | 100% | 100% |

Examples 117-122 describe the liquid formulation of pitolisant hydrochloride, that could be prepared using ingredients detailed in Table 48. The formulations could be prepared by dissolving, where applicable, sucralose, disodium edetate, benzalkonium chloride, n-dodecyl beta-D-maltoside, one-by-one, in about 80% of the required quantity of purified water. pH is adjusted between 4 and 6 using sodium hydroxide and/or hydrochloric acid. Where applicable, Poloxamer is added and mixed until dissolved and homogenized. Pitolisant hydrochloride is added and mixed until dissolved. Volume is made up to the batch size using purified water. The solution is optionally filtered and filled into containers such as plastic bottles or glass bottles and closed with closures. These formulations are suitable for intranasal administration with sustained delivery of pitolisant.

TABLE 48
Liquid formulation of pitolisant for intranasal use.

| | Example No. - Quantity (% w/v) | | | | | |
|---|---|---|---|---|---|---|
| Ingredient | 117 | 118 | 119 | 120 | 121 | 122 |
| Pitolisant | 10-40% | 10-40% | 10-40% | 10-40% | 20% | 20% |
| hydrochloride | | | | | | |
| Sucralose | 1-5% | 1-5% | 1-5% | 1-5% | 2% | 2% |
| Benzalkonium chloride | 0.1-0.2% | 0.1-0.2% | 0.1-0.2% | 0.1-0.2% | 0.1% | 0.1% |
| Disodium edetate | 0.1-0.2% | 0.1-0.2% | 0.1-0.2% | 0.1-0.2% | 0.15% | 0.15% |
| Poloxamer 188 | 0.05-5% | — | 0.05-5% | — | 0.2% | — |
| Poloxamer 407 | — | 0.05-5% | — | 0.05-5% | — | 0.2% |
| n-dodecyl beta-D- | — | — | 0.1-0.5% | 0.1-0.5% | — | — |
| maltoside | | | | | | |
| Sodium | q.s. to pH 4 | q.s. to pH 4 | q.s. to pH 4 | q.s. to pH 4 | q.s. to pH 4 | q.s. to pH 4 |
| hydroxide/hydrochloric | to 6 | to 6 | to 6 | to 6 | to 6 | to 6 |
| acid (optional) | | | | | | |
| Purified Water | q.s. to | q.s. to | q.s. to | q.s. to | q.s. to | q.s. to |
| | 100% | 100% | 100% | 100% | 100% | 100% |

Examples 123-127 describe the liquid formulation of pitolisant hydrochloride, that could be prepared using ingredients detailed in Table 49. The formulations could be prepared using procedure similar described earlier. This formulation could be suitable for oral administration. These formulations could have comparable pharmacokinetic profile with oral tablet dosage forms.

TABLE 49
Liquid formulation of pitolisant for oral use.

| | Example No. - Quantity (% w/v) | | | | |
|---|---|---|---|---|---|
| Ingredient | 123 | 124 | 125 | 126 | 127 |
| Pitolisant hydrochloride | 0.4-4% | 0.4-4% | 0.4-4% | 0.4-4% | 0.4-4% |
| Sucralose | 0.5-5% | 0.5-5% | 0.5-5% | 0.5-5% | 0.5-5% |
| Sodium benzoate | 0.05-1% | 0.05-1% | 0.05-1% | 0.05-1% | 0.05-1% |
| Cherry flavor | 0.05-1% | 0.05-1% | 0.05-1% | 0.05-1% | 0.05-1% |
| Hypromellose (HPMC K4M) | 0.5-5% | — | — | — | 0.1% |
| Xanthan gum | — | 0.01-2% | — | — | — |
| Poloxamer 407 | — | — | 0.5-5% | — | — |
| Poloxamer 188 | — | — | — | 1-5% | — |
| Polyviylpyrrolidone (PVP K-30) | — | — | — | — | 2-10% |
| Purified Water | q.s. to 100% | q.s. to 100% | q.s. to 100% | q.s. to 100% | q.s. to 100% |

Examples 128-135 describe liquid fill formulations intended for encapsulation within soft gelatin capsules, designed for either immediate or delayed release (achieved via capsule shell properties or enteric coating). The required quantity of the primary vehicle (e.g., propylene glycol, polyethylene glycol 400, or castor oil) is added to a suitable vessel. Where applicable, the antioxidant (e.g., α-tocopherol) is added and dissolved with mixing. Where applicable, the alkaline agent (e.g., tromethamine) is added and dissolved or dispersed with mixing until uniform. Pitolisant hydrochloride is then added and mixed until completely dissolved. The fill formulation is deaerated or overlayed with inert gas such as nitrogen if necessary before encapsulation. The prepared liquid fill is encapsulated into soft gelatin capsules of the appropriate size and composition (standard gelatin or modified shells for delayed release) using standard rotary die encapsulation equipment. Capsules are dried to the target shell hardness. For delayed release, capsules with standard shells could be further coated with an enteric polymer system. When ingredients are suspended, suspending agent and/or viscosity adjusting agents are recommended to use for physical stability of suspension.

TABLE 50
Liquid formulation of pitolisant for oral use.

| | Example No. - Quantity (% w/v) | | | | | | | |
|---|---|---|---|---|---|---|---|---|
| Ingredient | 128 | 129 | 130 | 131 | 132 | 133 | 134 | 135 |
| Pitolisant hydrochloride | 10- | 10- | 10- | 10- | 10- | 10- | 10- | 10- |
| | 50% | 50% | 50% | 50% | 50% | 50% | 50% | 50% |
| Tromethamine (micronized) | — | — | — | — | 0.1- | 0.1- | — | — |
| | | | | | 2% | 2% | | |
| Magnesium carbonate | — | — | — | — | — | — | 0.1- | 0.1- |
| (micronized) | | | | | | | 2% | 2% |
| α-Tocopherol (optional) | 0.05- | 0.05- | 0.05- | 0.05- | 0.05- | 0.05- | 0.05- | 0.05- |
| | 1% | 1% | 1% | 1% | 1% | 1% | 1% | 1% |
| Propylene glycol | q.s. to | — | — | 50% | q.s. to | — | q.s. to | — |
| | 100% | | | | 100% | | 100% | |
| Glycerin | — | q.s. to | — | q.s. to | — | q.s. to | — | q.s. to |
| | | 100% | | 100% | | 100% | | 100% |
| Castor oil | — | — | q.s. to | — | — | — | — | — |
| | | | 100% | | | | | |

### HPLC Analytical Method

An exemplary High-Performance Liquid Chromatography (HPLC) method is provided for use in analyzing pitolisant liquid formulations as described in the present invention. This method can be used to assess the purity of the formulation and detect impurities such as the N-oxide derivative of pitolisant. The results of such analysis are expressed as the percentage area of individual peaks observed in the chromatogram. It is understood that there is no requirement to use only this specific HPLC method; any other analytical approach that can provide information regarding the purity and impurities in pitolisant liquid formulations of the present invention may also be used.

TABLE 51
HPLC analytical method for testing pitolisant liquid formulation of present invention.

| HPLC instrument | Agilent 1260 Infinity III with PDA detector | | | | | | |
|---|---|---|---|---|---|---|---|
| Column specification | InertSustain C18 HPLC Column, 5 μm, 250 × 4.6 mm (GL | | | | | | |
| | Sciences/PN: 5020-07346) or Equivalent | | | | | | |
| Flow rate | 1.0 mL/min | | | | | | |
| Wavelength | 210 nm | | | | | | |
| Column temperature | 55° C. | | | | | | |
| Injection volume | 10 μL | | | | | | |
| Sample concentration | 1.0 mg/mL | | | | | | |
| Working standard | 1.0 mg/mL | | | | | | |
| concentration | | | | | | | |
| PTS N-Oxide impurity | 10 μg/mL | | | | | | |
| standard concentration | | | | | | | |
| Diluent | Water:Acetonitrile (75:25% v/v) or Water:Methanol (50:50% v/v) | | | | | | |
| Buffer | Dissolve 8.57 gm of anhydrous sodium perchlorate in 1.0 L of | | | | | | |
| | milli-Q water. Adjust pH to 2.20 with diluted perchloric acid (~7%), | | | | | | |
| | filter through 0.22μ PES filter. | | | | | | |
| Mobile Phase A | Buffer:Acetonitrile (75:25% v/v) | | | | | | |
| Mobile Phase B | Methanol:Water (90:100% v/v) | | | | | | |
| Run time | 68 minutes | | | | | | |
| Multisampler | 10° C. | | | | | | |
| temperature | | | | | | | |
| Gradient elution | Time (min) | 0 | 40 | 50 | 57 | 58 | 68 |
| | Mobile Phase A: | 72 | 69 | 55 | 55 | 72 | 72 |
| | Mobile Phase B: | 28 | 31 | 45 | 45 | 28 | 28 |

1. A stable liquid pharmaceutical formulation comprising:
(a) pitolisant or a pharmaceutically acceptable salt thereof; and
(b) at least one pharmaceutically acceptable excipients selected from the group comprising vehicles, preservatives, sweeteners, flavors, pH adjusting agents, buffering agents, viscosity adjusting agents, antioxidants, tonicity adjusting agents, chelating agents, permeability enhancing agents, mucoadhesive agents, stabilizers, complexing agents, crystallization inhibitors, suspending agents, surfactants, anti-foaming agents, dispersing agents, wetting agents, emulsifying agents, and combinations thereof,
wherein upon storage for at least three months at room temperature, the formulation has:
(i) at least about 99% pure pitolisant,
(ii) less than about 1% of total degradant impurities.

2. The formulation according to claim 1, wherein the salt is pitolisant hydrochloride.

3. The formulation according to claim 1, wherein the pitolisant concentration is from about 1 mg/mL to about 400 mg/mL.

4. The formulation according to claim 1, wherein the pH of the formulation is about 2 to about 7.

5. The formulation according to claim 1, wherein the formulation contains less than about 0.75% pitolisant N-oxide impurity.

6. The formulation according to claim 1, wherein the formulation is suitable for administration selected from oral, sublingual, buccal or intranasal routes.

7. The formulation according to claim 1, formulated as an oral liquid comprising pitolisant hydrochloride about 0.4-10% w/v, sucralose about 0.01-1%, sodium benzoate about 0.01-0.5%, hypromellose about 0.01-1%, at pH about 3-5.

8. The formulation according to claim 1, formulated as a nasal spray comprising pitolisant hydrochloride about 40-400 mg/mL, benzalkonium chloride, sucralose, and disodium edetate, at pH about 4-6.

9. The formulation according to claim 1, wherein the formulation is filled in a multi-dose container suitable for oral liquid administration.

10. The formulation according to claim 9, wherein the container is a glass or polymeric bottle of 5-500 mL fill volume.

11. The formulation according to claim 9, wherein the container is provided with a flow-restrictor and/or dosing syringe.

12. The formulation according to claim 1, wherein the formulation is filled in a container equipped with a metered-dose spray device suitable for oral, sublingual, buccal, or intranasal delivery.

13. The formulation according to claim 12, wherein each spray delivers 0.05-0.5 mL of formulation corresponding to about 1-200 mg of pitolisant hydrochloride.

14. The formulation according to claim 12, wherein each spray delivers 0.05-0.15 mL of formulation corresponding to about 10-100 mg of pitolisant hydrochloride.

15. The formulation according to claim 12, wherein the sprayer is adapted for unit-dose, bi-dose or multiple-dose delivery.

16. A method for treating one or more conditions selected from narcolepsy, excessive daytime sleepiness (EDS) associated with narcolepsy, cataplexy associated with narcolepsy, EDS associated with obstructive sleep apnea, idiopathic hypersomnia, Prader-Willi Syndrome, Parkinson's disease, Attention-deficit hyperactivity disorder (ADHD), schizophrenia, depression, cognitive impairment, myotonic dystrophy, Dravet syndrome, Lennox-Gastaut syndrome, Rett syndrome, Tourette syndrome, and binge-eating disorder, comprising administering a therapeutically effective amount of the formulation according to claim 1.

17. The method according to claim 16, wherein the condition is narcolepsy, excessive daytime sleepiness associated with narcolepsy, cataplexy associated with narcolepsy, Prader-Willi Syndrome, or excessive daytime sleepiness in patients with Prader-Willi syndrome.

18. The method according to claim 16, wherein the formulation is administered orally in a volume of about 0.1 mL to about 40 mL to deliver about 5 mg to about 200 mg of pitolisant per dose.

19. The method according to claim 16, wherein the formulation is administered as a spray intranasally, wherein each spray comprises about 0.05 mL to about 0.15 mL to deliver about 1 mg to about 90 mg of pitolisant per spray.

20. A method of preparing the liquid pharmaceutical formulation according to claim 1, the method comprising the steps of: (i) solubilizing pitolisant or its pharmaceutically acceptable salt in a vehicle; (ii) solubilizing one or more optional pharmaceutically acceptable excipients; (iii) optionally filtering the formulated liquid; (iv) filling the filtered liquid into a primary packaging container; and (v) sealing the container with an optional inert gas overlay.
